SEQUENCE LISTING (74 sequences)

SEQ ID NO: 1 - DNA, 1593 bp, Streptomyces coelicolor
atgagtgaacctgaagaacaacaacctgacatacatacaactgctggtaaattagccgacttgagaagaagaatagaagaagccacacatgctggtagtgcaagagccgttgaaaaacaacacgccaagggtaaattgactgctagagaaagaatcgatttgttattggacgaaggtagttttgttgaattagatgaattcgcaagacatagatctacaaattttggtttggatgccaacagaccatatggtgacggtgttgtcactggttacggtacagttgatggtagacctgttgctgtcttttctcaagactttacagttttcggtggtgcattaggtgaagtctatggtcaaaagattgtaaaggttatggatttcgctttgaaaaccggttgtccagtagttggtataaatgattcaggtggtgcaagaatccaagaaggtgttgcttccttaggtgcatacggtgaaatttttagaagaaacactcacgcttcaggtgtcatcccacaaatttccttggtcgtaggtccttgcgcaggtggtgccgtatactcaccagcaatcaccgattttactgtcatggtagaccaaacatcccatatgttcattaccggtcctgatgtcataaagactgtaacaggtgaagacgttggttttgaagaattgggtggtgctagaacccacaattctacttcaggtgtcgcccatcacatggctggtgacgaaaaagacgcagtcgaatacgtaaagcaattgttgtcctacttaccaagtaacaacttgtctgaaccacctgctttccctgaagaagctgatttggcagtaactgatgaagacgctgaattggatacaatcgttccagacagtgcaaaccaaccttacgatatgcattctgttattgaacacgtcttagatgacgccgaatttttcgaaacccaaccattatttgctcctaatatcttgactggtttcggtagagttgaaggtagaccagttggtattgtcgcaaaccaacctatgcaatttgccggttgtttggatataacagcttcagaaaaagctgcaagatttgttagaacatgcgatgctttcaatgttccagtcttaacctttgtagatgttccaggtttcttgcctggtgttgatcaagaacatgacggtataatcagaagaggtgctaagttgatatttgcttatgcagaagccactgtaccattgataacagttatcaccagaaaagcattcggtggtgcctacgatgttatgggttccaagcatttgggtgctgacttaaatttggcatggcctacagcccaaatagctgtcatgggtgcacaaggtgccgtaaacatcttacacagaagaactattgctgatgcaggtgacgacgccgaagctacaagagcaagattgatccaagaatacgaagatgccttgttgaacccatacaccgccgctgaaagaggttacgtagatgctgttattatgccttcagacactagaagacatatagttagaggtttgagacaattaagaaccaaaagagaatccttgccaccaaagaagcacggtaacatcccattataa

SEQ ID NO: 2 - DNA, 1773 bp, Streptomyces coelicolor
ttagtccttgatttcacagatagcagcaccagaggttaatgaggcaccaacttcagcagttaaacccttgatagtaccggatctatgtgcgttcaaaggttgttccatcttcatagcttccaaaactacgatcaagtcaccctcttgaacttcttgaccttcttcaactgcgatctttactatagtaccttgcataggtgaagctaaggtgtcaccggaggctgctggacctgactttttagcggctcttcttttaggtctagcacctgcagccaaaccggttctggctaaagacatgcccaaagaacttggtaaacttacttccaatcttttaccaccgacttcgacaactactgtttctctaccagattcttcatcggtttctgtgtcagctggtgtagtgaaaggtttaatttcgttaacgaattcagtttcgatccatcttgtgtgtacagtgaaaggatcggtactacctgttaattctggggcgaatgcagggtctctgacaactgttctatgaaatggaatagcggtggccataccctcaacagtgaattcatccaaagctctggctgctctttgtaatgcttcagctctagttctaccggtaactattaatttggctaacaaggaatcccatgctggaccaattacggaacctgattcgacaccagcgtccaatctgacacctggaccagtaggtgcatcgaataaagtaacggtacctggagcaggcaagaaacctctaccaggatcttcaccgttaattctgaattcaaatgagtgacctctcaatgctgggtcatcgtaacctaattcttcaccgtcagcaattctgaacatttctctaaccaaatcgataccagcgacttcttctgtaactggatgttctacttgcaatcttgtgttaacttctaagaaaaatatagtaccatccataccgaccaagaattcaacggtacctgcaccaccgtaaccagcttccttcaatatggccttggatgaagagtacaattgttctgtttgtgcttcagataaaaatggggcaggtgcttcttcaaccaacttttgatgtcttctttgcaaagaacaatctctagtacttacgacaactacgttaccgtgagtgtcagctaagcattgggtttcgacatgtcttggtttatccaagtatctttcaacgaaacattcaccacgaccaaaagcggctactgcttctctgacagcagaatcatacaattcaggaacttcttctaaagttcttgctaccttcaaacctctaccaccaccaccgaatgcagccttaatagcgattggtaaaccatgttctttagcaaaggcgacaacttcatcggcaccactgactgggtcaggtgtacctgcaaccaaaggtgcaccagctctttgagcgatgtgtctggctgcaactttttcacctctatctcttatagcatgtggaggtggacctatccaaattaaaccggcgtccaaaacggcttgtgcgaattctgcattttcggacaagaaaccgtaacctgggtgaatagcatcggcacctgattctctagcggccttcaaaacctttgcgatgtccaaataagatgtagcaggagtgtcaccacctaatgcaaaagcttcgtctgcagctctaacatgcaaagcatctctgtctggatcggcatagacggctacggaagcgataccagcgtctctacatgctcttgctactctaacggcgatttcacctctgtttgcgatcaatacctttctcat

SEQ ID NO: 3 - protein, 530 aa, Streptomyces coelicolor
MSEPEEQQPDIHTTAGKLADLRRRIEEATHAGSARAVEKQHAKGKLTARERIDLLLDEGSFVELDEFARHRSTNFGLDANRPYGDGVVTGYGTVDGRPVAVFSQDFTVFGGALGEVYGQKIVKVMDFALKTGCPVVGINDSGGARIQEGVASLGAYGEIFRRNTHASGVIPQISLVVGPCAGGAVYSPAITDFTVMVDQTSHMFITGPDVIKTVTGEDVGFEELGGARTHNSTSGVAHHMAGDEKDAVEYVKQLLSYLPSNNLSEPPAFPEEADLAVTDEDAELDTIVPDSANQPYDMHSVIEHVLDDAEFFETQPLFAPNILTGFGRVEGRPVGIVANQPMQFAGCLDITASEKAARFVRTCDAFNVPVLTFVDVPGFLPGVDQEHDGIIRRGAKLIFAYAEATVPLITVITRKAFGGAYDVMGSKHLGADLNLAWPTAQIAVMGAQGAVNILHRRTIADAGDDAEATRARLIQEYEDALLNPYTAAERGYVDAVIMPSDTRRHIVRGLRQLRTKRESLPPKKHGNIPL

SEQ ID NO: 4 - protein, 590 aa, Streptomyces coelicolor
MRKVLIANRGEIAVRVARACRDAGIASVAVYADPDRDALHVRAADEAFALGGDTPATSYLDIAKVLKAARESGADAIHPGYGFLSENAEFAQAVLDAGLIWIGPPPHAIRDRGEKVAARHIAQRAGAPLVAGTPDPVSGADEVVAFAKEHGLPIAIKAAFGGGGRGLKVARTLEEVPELYDSAVREAVAAFGRGECFVERYLDKPRHVETQCLADTHGNVVVVSTRDCSLQRRHQKLVEEAPAPFLSEAQTEQLYSSSKAILKEAGYGGAGTVEFLVGMDGTIFFLEVNTRLQVEHPVTEEVAGIDLVREMFRIADGEELGYDDPALRGHSFEFRINGEDPGRGFLPAPGTVTLFDAPTGPGVRLDAGVESGSVIGPAWDSLLAKLIVTGRTRAEALQRAARALDEFTVEGMATAIPFHRTVVRDPAFAPELTGSTDPFTVHTRWIETEFVNEIKPFTTPADTETDEESGRETVVVEVGGKRLEVSLPSSLGMSLARTGLAAGARPKRRAAKKSGPAASGDTLASPMQGTIVKIAVEEGQEVQEGDLIVVLEAMKMEQPLNAHRSGTIKGLTAEVGASLTSGAAICEIKD

SEQ ID NO: 5 - DNA, 36 bp, Artificial Sequence
  primer
cgtagcggaattcatgtcttttagcgaattttatca

SEQ ID NO: 6 - DNA, 31 bp, Artificial Sequence
  primer
tattagggagctcttactcttccatcgcctg

SEQ ID NO: 7 - DNA, 1887 bp, Escherichia coli
atgtcttttagcgaattttatcagcgttcgattaacgaaccggagcagttctgggccgagcaggcccggcgtattgactggcagacgccctttacgcaaacgctcgatcacagcaatccgccgtttgcccgttggttttgtgaaggccgaaccaacttgtgccacaacgccatcgaccgctggctggagaaacagccagaggcgctggcgctgattgccgtctcttcggaaacagaagaagagcgcacctttacctttcgtcagctgcatgacgaagtgaacgcggtggcctcaatgttgcgttcattgggtgtgcagcgcggcgatcgggtgctggtgtatatgccgatgattgccgaagcgcatattactctgctggcctgcgcgcgcattggcgctattcactcggtggtgtttggtggatttgcctcgcacagcgtggcggcgcgaattgatgacgctaaaccggtgctgattgtctcggctgatgccggagcgcgcggtggcaaaatcattccctataaaaaattgctcgacgatgcgataagtcaggcgcagcaccagccacgccatgttttgctggtggatcgcgggctggcgaaaatggcgcgcgtcagcgggcgggatgtcgatttcgcgtcgttgcgccatcaacacatcggcgcgcgggtaccggtggcgtggctggaatccaacgaaacctcctgcattctctacacttccggcacgaccggcaaacctaaaggcgtgcagcgtgacgtcggcggatatgcggtggcgctggcgacctcgatggacaccatttttggcggcaaagcgggcagcgtgttcttttgcgcatcggatatcggctgggtggtggggcattcgtatatcgtttacgcgccgctgctggcggggatggcgactatcgtttacgaaggattgccgacctggccggactgcggcgtgtggtggacaatcgtcgagaaatatcaggttagccggatgttctcagcgccgaccgccattcgcgtgctgaaaaaattccctaccgctgaaattcgcaaacacgatctctcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggacgacaggccgacgcgtctgggaagccccggtgtgccgatgtatggctataacgtgcagttgcttaatgaagtcaccggcgaaccgtgtggcgtcaacgagaaagggatgctggtggtggaagggccgctgccgccggggtgtattcagaccatctggggcgacgacggccgctttgtgaagacttactggtcgctgttttcccgcccggtgtacgccacctttgactggggcatccgtgacgctgacggttatcactttattctcgggcgcactgacgatgtaattaacgttgccgggcatcggctggggacgcgcgagattgaagagagtatctccagccatccgggcgttgccgaagtggcggtggttggggtgaaagatgcgctgaaagggcaggtggcggtggcgtttgtcattccgaaagagagcgacagtctggaagatcgtgatgtggcgcactcgcaagagaaggcgattatggcgctggtggacagccagattggcaactttggccgcccggcgcacgtctggtttgtctcgcaattgccaaaaacgcgatccggaaaaatgctgcgccgcacgatccaggcgatttgcgaaggacgcgatcctggagatctgacgaccattgatgatcctgcgtcgttggatcagatccgccaggcgatggaagagtag

SEQ ID NO: 8 - protein, 628 aa, Escherichia coli [sequence omitted]

SEQ ID NO: 9 - DNA, 1413 bp, Saccharomyces cerevisiae
atgaatgtcaccgacgcaccacctgtgctatttaccgttcaagatacagctagagttatcacgctaaataggcccaaaaagctcaatgctttgaacgccgaaatgtcagaatccatgttcaagactttgaacgagtatgcaaagagcgatactacaaacttagtcattttaaagtcatccaaccgaccacgttcgttctgtgctggtggtgatgtagctactgtggcaatattcaattttaacaaagaatttgccaagtccatcaaattttttactgatgaatattctttgaattttcaaatagcaacttacttgaaaccaattgttaccttcatggacggtatcaccatgggtggcggcgttggtctatccattcacacgccctttagaattgctacagaaaacaccaaatgggccatgcccgagatggacattggtttttttccagatgtaggctcaacttttgctctccctagaatcgtgacattggctaactcaaactcacaaatggccctgtatctatgtcttacaggagaagtagtcacaggagcagacgcttatatgctcggcttagcgtctcattacgtcagtagtgaaaatttagatgctttgcagaaaagattaggtgaaattagccccccttttaataacgatccacaatctgcatacttcttcgggatggttaacgaatccatcgacgaattcgtatcaccattaccaaaagattatgttttcaagtattctaacgagaaattaaacgttattgaagcctgttttaacttgtctaaaaatggtactattgaagacataatgaataacttacgtcaatatgaaggttctgcggaaggtaaggctttcgcacaagaaatcaaaacgaaattgttaaccaagtcaccatcctctcttcaaatcgccttgagattggtgcaagagaattccagagatcacatagaatctgctatcaaaagagacttatacacagcagctaacatgtgcatgaaccaggactctttggtggaattctctgaagccacaaagcataaacttattgataaacaaagggtcccgtatccatggacaaagaaggaacagttatttgtatctcagttgacatctatcacatctcctaaaccatcgctaccaatgtcattactaagaaatacctcgaatgttacttggactcaatatccctaccattctaaataccaattgcctacagaacaggaaatcgctgcgtatattgaaaagagaacgaatgatgacactggcgccaaagttaccgaaagagaagtactaaatcactttgccaatgtgattccttctagaagagggaaactgggtatccaatcgctatgtaaaattgtttgtgaaagaaaatgtgaagaagttaacgatggcttaagatggaaataa

SEQ ID NO: 10 - protein, 470 aa, Saccharomyces cerevisiae
MNVTDAPPVLFTVQDTARVITLNRPKKLNALNAEMSESMFKTLNEYAKSDTTNLVILKSSNRPRSFCAGGDVATVAIFNFNKEFAKSIKFFTDEYSLNFQIATYLKPIVTFMDGITMGGGVGLSIHTPFRIATENTKWAMPEMDIGFFPDVGSTFALPRIVTLANSNSQMALYLCLTGEVVTGADAYMLGLASHYVSSENLDALQKRLGEISPPFNNDPQSAYFFGMVNESIDEFVSPLPKDYVFKYSNEKLNVIEACFNLSKNGTIEDIMNNLRQYEGSAEGKAFAQEIKTKLLTKSPSSLQIALRLVQENSRDHIESAIKRDLYTAANMCMNQDSLVEFSEATKHKLIDKQRVPYPWTKKEQLFVSQLTSITSPKPSLPMSLLRNTSNVTWTQYPYHSKYQLPTEQEIAAYIEKRTNDDTGAKVTEREVLNHFANVIPSRRGKLGIQSLCKIVCERKCEEVNDGLRWK

SEQ ID NO: 11 - DNA, 31 bp, Artificial Sequence
  primer
cgagtaggatccatgaatgtcaccgacgcac

SEQ ID NO: 12 - DNA, 34 bp, Artificial Sequence
  primer
tactattctcgagttatttccatcttaagccatc

SEQ ID NO: 13 - DNA, 2145 bp, Escherichia coli
atgtctaacgtgcaggagtggcaacagcttgccaacaaggaattgagccgtcgggagaaaactgtcgactcgctggttcatcaaaccgcggaagggatcgccatcaagccgctgtataccgaagccgatctcgataatctggaggtgacaggtacccttcctggtttgccgccctacgttcgtggcccgcgtgccactatgtataccgcccaaccgtggaccatccgtcagtatgctggtttttcaacagcaaaagagtccaacgctttttatcgccgtaacctggccgccgggcaaaaaggtctttccgttgcgtttgaccttgccacccaccgtggctacgactccgataacccgcgcgtggcgggcgacgtcggcaaagcgggcgtcgctatcgacaccgtggaagatatgaaagtcctgttcgaccagatcccgctggataaaatgtcggtttcgatgaccatgaatggcgcagtgctaccagtactggcgttttatatcgtcgccgcagaagagcaaggtgttacacctgataaactgaccggcaccattcaaaacgatattctcaaagagtacctctgccgcaacacctatatttacccaccaaaaccgtcaatgcgcattatcgccgacatcatcgcctggtgttccggcaacatgccgcgatttaataccatcagtatcagcggttaccacatgggtgaagcgggtgccaactgcgtgcagcaggtagcatttacgctcgctgatgggattgagtacatcaaagcagcaatctctgccggactgaaaattgatgacttcgctcctcgcctgtcgttcttcttcggcatcggcatggatctgtttatgaacgtcgccatgttgcgtgcggcacgttatttatggagcgaagcggtcagtggatttggcgcacaggacccgaaatcactggcgctgcgtacccactgccagacctcaggctggagcctgactgaacaggatccgtataacaacgttatccgcaccaccattgaagcgctggctgcgacgctgggcggtactcagtcactgcataccaacgcctttgacgaagcgcttggtttgcctaccgatttctcagcacgcattgcccgcaacacccagatcatcatccaggaagaatcagaactctgccgcaccgtcgatccactggccggatcctattacattgagtcgctgaccgatcaaatcgtcaaacaagccagagctattatccaacagatcgacgaagccggtggcatggcgaaagcgatcgaagcaggtctgccaaaacgaatgatcgaagaggcctcagcgcgcgaacagtcgctgatcgaccagggcaagcgtgtcatcgttggtgtcaacaagtacaaactggatcacgaagacgaaaccgatgtacttgagatcgacaacgtgatggtgcgtaacgagcaaattgcttcgctggaacgcattcgcgccacccgtgatgatgccgccgtaaccgccgcgttgaacgccctgactcacgccgcacagcataacgaaaacctgctggctgccgctgttaatgccgctcgcgttcgcgccaccctgggtgaaatttccgatgcgctggaagtcgctttcgaccgttatctggtgccaagccagtgtgttaccggcgtgattgcgcaaagctatcatcagtctgagaaatcggcctccgagttcgatgccattgttgcgcaaacggagcagttccttgccgacaatggtcgtcgcccgcgcattctgatcgctaagatgggccaggatggacacgatcgcggcgcgaaagtgatcgccagcgcctattccgatctcggtttcgacgtagatttaagcccgatgttctctacacctgaagagatcgcccgcctggccgtagaaaacgacgttcacgtagtgggcgcatcctcactggctgccggtcataaaacgctgatcccggaactggtcgaagcgctgaaaaaatggggacgcgaagatatctgcgtggtcgcgggtggcgtcattccgccgcaggattacgccttcctgcaagagcgcggcgtggcggcgatttatggtccaggtacacctatgctcgacagtgtgcgcgacgtactgaatctgataagccagcatcatgattaa

SEQ ID NO: 14 - protein, 714 aa, Escherichia coli
MSNVQEWQQLANKELSRREKTVDSLVHQTAEGIAIKPLYTEADLDNLEVTGTLPGLPPYVRGPRATMYTAQPWTIRQYAGFSTAKESNAFYRRNLAAGQKGLSVAFDLATHRGYDSDNPRVAGDVGKAGVAIDTVEDMKVLFDQIPLDKMSVSMTMNGAVLPVLAFYIVAAEEQGVTPDKLTGTIQNDILKEYLCRNTYIYPPKPSMRIIADIIAWCSGNMPRFNTISISGYHMGEAGANCVQQVAFTLADGIEYIKAAISAGLKIDDFAPRLSFFFGIGMDLFMNVAMLRAARYLWSEAVSGFGAQDPKSLALRTHCQTSGWSLTEQDPYNNVIRTTIEALAATLGGTQSLHTNAFDEALGLPTDFSARIARNTQIIIQEESELCRTVDPLAGSYYIESLTDQIVKQARAIIQQIDEAGGMAKAIEAGLPKRMIEEASAREQSLIDQGKRVIVGVNKYKLDHEDETDVLEIDNVMVRNEQIASLERIRATRDDAAVTAALNALTHAAQHNENLLAAAVNAARVRATLGEISDALEVAFDRYLVPSQCVTGVIAQSYHQSEKSASEFDAIVAQTEQFLADNGRRPRILIAKMGQDGHDRGAKVIASAYSDLGFDVDLSPMFSTPEEIARLAVENDVHVVGASSLAAGHKTLIPELVEALKKWGREDICVVAGGVIPPQDYAFLQERGVAAIYGPGTPMLDSVRDVLNLISQHHD

SEQ ID NO: 15 - DNA, 30 bp, Artificial Sequence
  primer
tgacagagatctatgtctaacgtgcaggag

SEQ ID NO: 16 - DNA, 35 bp, Artificial Sequence
  primer
cagtgactctcgagttaatcatgatgctggcttat

SEQ ID NO: 17 - DNA, 405 bp, Rhodobacter sphaeroides
atgattggacgtttgaatcatgttgccattgcggttccggatctggaagcggcggctgcgcaataccgtaatacgttgggcgctgaagtaggcgccccccaggatgaacccgatcatggcgttaccgtaatttttattacgttacctaacacaaaaattgaactgctccacccgcttggcgaaggttcacccatcgcagggtttctcgaaaaaaatccggcaggcgggattcatcacatctgttacgaggtcgaagatatcctggccgctcgtgatcgtctgaaagaagcgggtgcccgtgttttgggcagcggcgagcccaagattggtgcgcacggaaaaccggttctcttccttcaccctaaggactttaatggttgcctggtagaactggaacaggtgtaa

SEQ ID NO: 18 - DNA, 465 bp, Haemophilus influenzae
atgtctgccaactttactgataagaacggtcgtcaatctaaaggtgttttattgttgcgtactttggctatgccatccgataccaatgctaacggtgacattttcggtggttggattatgtctcaaatggacatgggtggtgctattttagctaaagaaatcgctcacggtcgtgttgtcactgttgccgttgaatctatgaactttattaagccaatttccgtcggtgacgttgtttgttgttacggtcaatgtttaaaggttggtagatcctccatcaagattaaggttgaagtctgggttaaaaaggttgcttccgaaccaatcggtgaacgttactgtgttactgacgccgtcttcacttttgtcgctgttgacaataatggtagatccagaactattccaagagaaaacaatcaagaattggaaaaggccttggctttgatttccgaacaaccattgtaa

SEQ ID NO: 19 - protein, 154 aa, Haemophilus influenzae
MSANFTDKNGRQSKGVLLLRTLAMPSDTNANGDIFGGWIMSQMDMGGAILAKEIAHGRVVTVAVESMNFIKPISVGDVVCCYGQCLKVGRSSIKIKVEVWVKKVASEPIGERYCVTDAVFTFVAVDNNGRSRTIPRENNQELEKALALISEQPL

SEQ ID NO: 20 - DNA, 399 bp, Escherichia coli
atgtctacaacacataacgtccctcagggcgatcttgttttacgtactttagccatgcccgccgataccaatgccaatggtgacatctttggtggttggttaatgtcacaaatggatattggcggcgctattctggcaaaagaaattgcccacggtcgcgtagtgactgtgcgggttgaaggaatgactttcttacggccggttgcggtcggcgatgtggtgtgctgctatgcacgctgtgtccagaaagggacgacatcggtcagcattaatattgaagtgtgggtgaaaaaagtagcgtctgaaccaattgggcaacgctataaagcgacagaagcattatttaagtatgtcgcggttgatcctgaaggaaaacctcgcgccttacctgttgagtaa

SEQ ID NO: 21 - protein, 132 aa, Escherichia coli
MSTTHNVPQGDLVLRTLAMPADTNANGDIFGGWLMSQMDIGGAILAKEIAHGRVVTVRVEGMTFLRPVAVGDVVCCYARCVQKGTTSVSINIEVWVKKVASEPIGQRYKATEALFKYVAVDPEGKPRALPVE

SEQ ID NO: 22 - DNA, 41 bp, Artificial Sequence
  primer
tgaataagatctaggatccatgtctacaacacataacgtcc

SEQ ID NO: 23 - DNA, 32 bp, Artificial Sequence
  primer
tctatctcgagttactcaacaggtaaggcgcg

SEQ ID NO: 24 - DNA, 39 bp, Artificial Sequence
  primer
ttttactgatagttattctttgaattttcaaatagcaac

SEQ ID NO: 25 - DNA, 23 bp, Artificial Sequence
  primer
aatttgatggacttggcaaattc

SEQ ID NO: 26 - DNA, 36 bp, Artificial Sequence
  primer
ttttactgatgtttattctttgaattttcaaatagc

SEQ ID NO: 27 - DNA, 26 bp, Artificial Sequence
  primer
ggacattggtctttttccagatgtag

SEQ ID NO: 28 - DNA, 18 bp, Artificial Sequence
  primer
atctcgggcatggcccat

SEQ ID NO: 29 - DNA, 31 bp, Artificial Sequence
  primer
gttaaccaaggctccatcctctcttcaaatc

SEQ ID NO: 30 - DNA, 22 bp, Artificial Sequence
  primer
aatttcgttttgatttcttgtg

SEQ ID NO: 31 - DNA, 1413 bp, Saccharomyces cerevisiae
atgaatgtcaccgacgcaccacctgtgctatttaccgttcaagatacagctagagttatcacgctaaataggcccaaaaagctcaatgctttgaacgccgaaatgtcagaatccatgttcaagactttgaacgagtatgcaaagagcgatactacaaacttagtcattttaaagtcatccaaccgaccacgttcgttctgtgctggtggtgatgtagctactgtggcaatattcaattttaacaaagaatttgccaagtccatcaaattttttactgatagttattctttgaattttcaaatagcaacttacttgaaaccaattgttaccttcatggacggtatcaccatgggtggcggcgttggtctatccattcacacgccctttagaattgctacagaaaacaccaaatgggccatgcccgagatggacattggtttttttccagatgtaggctcaacttttgctctccctagaatcgtgacattggctaactcaaactcacaaatggccctgtatctatgtcttacaggagaagtagtcacaggagcagacgcttatatgctcggcttagcgtctcattacgtcagtagtgaaaatttagatgctttgcagaaaagattaggtgaaattagccccccttttaataacgatccacaatctgcatacttcttcgggatggttaacgaatccatcgacgaattcgtatcaccattaccaaaagattatgttttcaagtattctaacgagaaattaaacgttattgaagcctgttttaacttgtctaaaaatggtactattgaagacataatgaataacttacgtcaatatgaaggttctgcggaaggtaaggctttcgcacaagaaatcaaaacgaaattgttaaccaagtcaccatcctctcttcaaatcgccttgagattggtgcaagagaattccagagatcacatagaatctgctatcaaaagagacttatacacagcagctaacatgtgcatgaaccaggactctttggtggaattctctgaagccacaaagcataaacttattgataaacaaagggtcccgtatccatggacaaagaaggaacagttatttgtatctcagttgacatctatcacatctcctaaaccatcgctaccaatgtcattactaagaaatacctcgaatgttacttggactcaatatccctaccattctaaataccaattgcctacagaacaggaaatcgctgcgtatattgaaaagagaacgaatgatgacactggcgccaaagttaccgaaagagaagtactaaatcactttgccaatgtgattccttctagaagagggaaactgggtatccaatcgctatgtaaaattgtttgtgaaagaaaatgtgaagaagttaacgatggcttaagatggaaataa

SEQ ID NO: 32 - protein, 470 aa, Saccharomyces cerevisiae
MNVTDAPPVLFTVQDTARVITLNRPKKLNALNAEMSESMFKTLNEYAKSDTTNLVILKSSNRPRSFCAGGDVATVAIFNFNKEFAKSIKFFTDSYSLNFQIATYLKPIVTFMDGITMGGGVGLSIHTPFRIATENTKWAMPEMDIGFFPDVGSTFALPRIVTLANSNSQMALYLCLTGEVVTGADAYMLGLASHYVSSENLDALQKRLGEISPPFNNDPQSAYFFGMVNESIDEFVSPLPKDYVFKYSNEKLNVIEACFNLSKNGTIEDIMNNLRQYEGSAEGKAFAQEIKTKLLTKSPSSLQIALRLVQENSRDHIESAIKRDLYTAANMCMNQDSLVEFSEATKHKLIDKQRVPYPWTKKEQLFVSQLTSITSPKPSLPMSLLRNTSNVTWTQYPYHSKYQLPTEQEIAAYIEKRTNDDTGAKVTEREVLNHFANVIPSRRGKLGIQSLCKIVCERKCEEVNDGLRWK

SEQ ID NO: 33 - DNA, 1413 bp, Saccharomyces cerevisiae
atgaatgtcaccgacgcaccacctgtgctatttaccgttcaagatacagctagagttatcacgctaaataggcccaaaaagctcaatgctttgaacgccgaaatgtcagaatccatgttcaagactttgaacgagtatgcaaagagcgatactacaaacttagtcattttaaagtcatccaaccgaccacgttcgttctgtgctggtggtgatgtagctactgtggcaatattcaattttaacaaagaatttgccaagtccatcaaattttttactgatgtttattctttgaattttcaaatagcaacttacttgaaaccaattgttaccttcatggacggtatcaccatgggtggcggcgttggtctatccattcacacgccctttagaattgctacagaaaacaccaaatgggccatgcccgagatggacattggtttttttccagatgtaggctcaacttttgctctccctagaatcgtgacattggctaactcaaactcacaaatggccctgtatctatgtcttacaggagaagtagtcacaggagcagacgcttatatgctcggcttagcgtctcattacgtcagtagtgaaaatttagatgctttgcagaaaagattaggtgaaattagccccccttttaataacgatccacaatctgcatacttcttcgggatggttaacgaatccatcgacgaattcgtatcaccattaccaaaagattatgttttcaagtattctaacgagaaattaaacgttattgaagcctgttttaacttgtctaaaaatggtactattgaagacataatgaataacttacgtcaatatgaaggttctgcggaaggtaaggctttcgcacaagaaatcaaaacgaaattgttaaccaagtcaccatcctctcttcaaatcgccttgagattggtgcaagagaattccagagatcacatagaatctgctatcaaaagagacttatacacagcagctaacatgtgcatgaaccaggactctttggtggaattctctgaagccacaaagcataaacttattgataaacaaagggtcccgtatccatggacaaagaaggaacagttatttgtatctcagttgacatctatcacatctcctaaaccatcgctaccaatgtcattactaagaaatacctcgaatgttacttggactcaatatccctaccattctaaataccaattgcctacagaacaggaaatcgctgcgtatattgaaaagagaacgaatgatgacactggcgccaaagttaccgaaagagaagtactaaatcactttgccaatgtgattccttctagaagagggaaactgggtatccaatcgctatgtaaaattgtttgtgaaagaaaatgtgaagaagttaacgatggcttaagatggaaataa

SEQ ID NO: 34 - protein, 470 aa, Saccharomyces cerevisiae
MNVTDAPPVLFTVQDTARVITLNRPKKLNALNAEMSESMFKTLNEYAKSDTTNLVILKSSNRPRSFCAGGDVATVAIFNFNKEFAKSIKFFTDVYSLNFQIATYLKPIVTFMDGITMGGGVGLSIHTPFRIATENTKWAMPEMDIGFFPDVGSTFALPRIVTLANSNSQMALYLCLTGEVVTGADAYMLGLASHYVSSENLDALQKRLGEISPPFNNDPQSAYFFGMVNESIDEFVSPLPKDYVFKYSNEKLNVIEACFNLSKNGTIEDIMNNLRQYEGSAEGKAFAQEIKTKLLTKSPSSLQIALRLVQENSRDHIESAIKRDLYTAANMCMNQDSLVEFSEATKHKLIDKQRVPYPWTKKEQLFVSQLTSITSPKPSLPMSLLRNTSNVTWTQYPYHSKYQLPTEQEIAAYIEKRTNDDTGAKVTEREVLNHFANVIPSRRGKLGIQSLCKIVCERKCEEVNDGLRWK

SEQ ID NO: 35 - DNA, 1413 bp, Saccharomyces cerevisiae
atgaatgtcaccgacgcaccacctgtgctatttaccgttcaagatacagctagagttatcacgctaaataggcccaaaaagctcaatgctttgaacgccgaaatgtcagaatccatgttcaagactttgaacgagtatgcaaagagcgatactacaaacttagtcattttaaagtcatccaaccgaccacgttcgttctgtgctggtggtgatgtagctactgtggcaatattcaattttaacaaagaatttgccaagtccatcaaattttttactgatgaatattctttgaattttcaaatagcaacttacttgaaaccaattgttaccttcatggacggtatcaccatgggtggcggcgttggtctatccattcacacgccctttagaattgctacagaaaacaccaaatgggccatgcccgagatggacattggtctttttccagatgtaggctcaacttttgctctccctagaatcgtgacattggctaactcaaactcacaaatggccctgtatctatgtcttacaggagaagtagtcacaggagcagacgcttatatgctcggcttagcgtctcattacgtcagtagtgaaaatttagatgctttgcagaaaagattaggtgaaattagccccccttttaataacgatccacaatctgcatacttcttcgggatggttaacgaatccatcgacgaattcgtatcaccattaccaaaagattatgttttcaagtattctaacgagaaattaaacgttattgaagcctgttttaacttgtctaaaaatggtactattgaagacataatgaataacttacgtcaatatgaaggttctgcggaaggtaaggctttcgcacaagaaatcaaaacgaaattgttaaccaagtcaccatcctctcttcaaatcgccttgagattggtgcaagagaattccagagatcacatagaatctgctatcaaaagagacttatacacagcagctaacatgtgcatgaaccaggactctttggtggaattctctgaagccacaaagcataaacttattgataaacaaagggtcccgtatccatggacaaagaaggaacagttatttgtatctcagttgacatctatcacatctcctaaaccatcgctaccaatgtcattactaagaaatacctcgaatgttacttggactcaatatccctaccattctaaataccaattgcctacagaacaggaaatcgctgcgtatattgaaaagagaacgaatgatgacactggcgccaaagttaccgaaagagaagtactaaatcactttgccaatgtgattccttctagaagagggaaactgggtatccaatcgctatgtaaaattgtttgtgaaagaaaatgtgaagaagttaacgatggcttaagatggaaataa

SEQ ID NO: 36 - protein, 470 aa, Saccharomyces cerevisiae
MNVTDAPPVLFTVQDTARVITLNRPKKLNALNAEMSESMFKTLNEYAKSDTTNLVILKSSNRPRSFCAGGDVATVAIFNFNKEFAKSIKFFTDEYSLNFQIATYLKPIVTFMDGITMGGGVGLSIHTPFRIATENTKWAMPEMDIGLFPDVGSTFALPRIVTLANSNSQMALYLCLTGEVVTGADAYMLGLASHYVSSENLDALQKRLGEISPPFNNDPQSAYFFGMVNESIDEFVSPLPKDYVFKYSNEKLNVIEACFNLSKNGTIEDIMNNLRQYEGSAEGKAFAQEIKTKLLTKSPSSLQIALRLVQENSRDHIESAIKRDLYTAANMCMNQDSLVEFSEATKHKLIDKQRVPYPWTKKEQLFVSQLTSITSPKPSLPMSLLRNTSNVTWTQYPYHSKYQLPTEQEIAAYIEKRTNDDTGAKVTEREVLNHFANVIPSRRGKLGIQSLCKIVCERKCEEVNDGLRWK

SEQ ID NO: 37 - DNA, 1413 bp, Saccharomyces cerevisiae
atgaatgtcaccgacgcaccacctgtgctatttaccgttcaagatacagctagagttatcacgctaaataggcccaaaaagctcaatgctttgaacgccgaaatgtcagaatccatgttcaagactttgaacgagtatgcaaagagcgatactacaaacttagtcattttaaagtcatccaaccgaccacgttcgttctgtgctggtggtgatgtagctactgtggcaatattcaattttaacaaagaatttgccaagtccatcaaattttttactgatgaatattctttgaattttcaaatagcaacttacttgaaaccaattgttaccttcatggacggtatcaccatgggtggcggcgttggtctatccattcacacgccctttagaattgctacagaaaacaccaaatgggccatgcccgagatggacattggtttttttccagatgtaggctcaacttttgctctccctagaatcgtgacattggctaactcaaactcacaaatggccctgtatctatgtcttacaggagaagtagtcacaggagcagacgcttatatgctcggcttagcgtctcattacgtcagtagtgaaaatttagatgctttgcagaaaagattaggtgaaattagccccccttttaataacgatccacaatctgcatacttcttcgggatggttaacgaatccatcgacgaattcgtatcaccattaccaaaagattatgttttcaagtattctaacgagaaattaaacgttattgaagcctgttttaacttgtctaaaaatggtactattgaagacataatgaataacttacgtcaatatgaaggttctgcggaaggtaaggctttcgcacaagaaatcaaaacgaaattgttaaccaaggctccatcctctcttcaaatcgccttgagattggtgcaagagaattccagagatcacatagaatctgctatcaaaagagacttatacacagcagctaacatgtgcatgaaccaggactctttggtggaattctctgaagccacaaagcataaacttattgataaacaaagggtcccgtatccatggacaaagaaggaacagttatttgtatctcagttgacatctatcacatctcctaaaccatcgctaccaatgtcattactaagaaatacctcgaatgttacttggactcaatatccctaccattctaaataccaattgcctacagaacaggaaatcgctgcgtatattgaaaagagaacgaatgatgacactggcgccaaagttaccgaaagagaagtactaaatcactttgccaatgtgattccttctagaagagggaaactgggtatccaatcgctatgtaaaattgtttgtgaaagaaaatgtgaagaagttaacgatggcttaagatggaaataa

SEQ ID NO: 38 - protein, 470 aa, Saccharomyces cerevisiae
MNVTDAPPVLFTVQDTARVITLNRPKKLNALNAEMSESMFKTLNEYAKSDTTNLVILKSSNRPRSFCAGGDVATVAIFNFNKEFAKSIKFFTDEYSLNFQIATYLKPIVTFMDGITMGGGVGLSIHTPFRIATENTKWAMPEMDIGFFPDVGSTFALPRIVTLANSNSQMALYLCLTGEVVTGADAYMLGLASHYVSSENLDALQKRLGEISPPFNNDPQSAYFFGMVNESIDEFVSPLPKDYVFKYSNEKLNVIEACFNLSKNGTIEDIMNNLRQYEGSAEGKAFAQEIKTKLLTKAPSSLQIALRLVQENSRDHIESAIKRDLYTAANMCMNQDSLVEFSEATKHKLIDKQRVPYPWTKKEQLFVSQLTSITSPKPSLPMSLLRNTSNVTWTQYPYHSKYQLPTEQEIAAYIEKRTNDDTGAKVTEREVLNHFANVIPSRRGKLGIQSLCKIVCERKCEEVNDGLRWK

SEQ ID NO: 39 - protein, 134 aa, Rhodobacter sphaeroides
MIGRLNHVAIAVPDLEAAAAQYRNTLGAEVGAPQDEPDHGVTVIFITLPNTKIELLHPLGEGSPIAGFLEKNPAGGIHHICYEVEDILAARDRLKEAGARVLGSGEPKIGAHGKPVLFLHPKDFNGCLVELEQV

SEQ ID NO: 40 - DNA, 1887 bp, Escherichia coli
atgtcttttagcgaattttatcagcgttcgattaacgaaccggagcagttctgggccgagcaggcccggcgtattgactggcagacgccctttacgcaaacgctcgatcacagcaatccgccgtttgcccgttggttttgtgaaggccgaaccaacttgtgccacaacgccatcgaccgctggctggagaaacagccagaggcgctggcgctgattgccgtctcttcggaaacagaagaagagcgcacctttacctttcgtcagctgcatgacgaagtgaacgcggtggcctcaatgttgcgttcattgggtgtgcagcgcggcgatcgggtgctggtgtatatgccgatgattgccgaagcgcatattactctgctggcctgcgcgcgcattggcgctattcactcggtggtgtttggtggatttgcctcgcacagcgtggcggcgcgaattgatgacgctaaaccggtgctgattgtctcggctgatgccggagcgcgcggtggcaaaatcattccctataaaaaattgctcgacgatgcgataagtcaggcgcagcaccagccacgccatgttttgctggtggatcgcgggctggcgaaaatggcgcgcgtcagcgggcgggatgtcgatttcgcgtcgttgcgccatcaacacatcggcgcgcgggtaccggtggcgtggctggaatccaacgaaacctcctgcattctctacacttccggcacgaccggcaaacctaaaggcgtgcagcgtgacgtcggcggatatgcggtggcgctggcgacctcgatggacaccatttttggcggcaaagcgggcagcgtgttcttttgcgcatcggatatcggctgggtggtggggcattcgtatatcgtttacgcgccgctgctggcggggatggcgactatcgtttacgaaggattgccgacctggccggactgcggcgtgtggtggacaatcgtcgagaaatatcaggttagccggatgttctcagcgccgaccgccattcgcgtgctgaaaaaattccctaccgctgaaattcgcaaacacgatctctcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggacgacaggccgacgcgtctgggaagccccggtgtgccgatgtatggctataacgtgcagttgcttaatgaagtcaccggcgaaccgtgtggcgtcaacgagaaagggatgctggtggtggaagggccgctgccgccggggtgtattcagaccatctggggcgacgacggccgctttgtgaagacttactggtcgctgttttcccgcccggtgtacgccacctttgactggggcatccgtgacgctgacggttatcactttattctcgggcgcactgacgatgtaattaacgttgccgggcatcggctggggacgcgcgagattgaagagagtatctccagccatccgggcgttgccgaagtggcggtggttggggtgaaagatgcgctgaaagggcaggtggcggtggcgtttgtcattccgaaagagagcgacagtctggaagatcgtgatgtggcgcactcgcaagagaaggcgattatggcgctggtggacagccagattggcaactttggccgcccggcgcacgtctggtttgtctcgcaattgccaaaaacgcgatccggagaaatgctgcgccgcacgatccaggcgatttgcgaaggacgcgatcctggagatctgacgaccattgatgatcctgcgtcgttggatcagatccgccaggcgatggaagagtag

SEQ ID NO: 41 - protein, 628 aa, Escherichia coli
MSFSEFYQRSINEPEQFWAEQARRIDWQTPFTQTLDHSNPPFARWFCEGRTNLCHNAIDRWLEKQPEALALIAVSSETEEERTFTFRQLHDEVNAVASMLRSLGVQRGDRVLVYMPMIAEAHITLLACARIGAIHSVVFGGFASHSVAARIDDAKPVLIVSADAGARGGKIIPYKKLLDDAISQAQHQPRHVLLVDRGLAKMARVSGRDVDFASLRHQHIGARVPVAWLESNETSCILYTSGTTGKPKGVQRDVGGYAVALATSMDTIFGGKAGSVFFCASDIGWVVGHSYIVYAPLLAGMATIVYEGLPTWPDCGVWWTIVEKYQVSRMFSAPTAIRVLKKFPTAEIRKHDLSSLEVLYLAGEPLDEPTASWVSNTLDVPVIDNYWQTESGWPIMAIARGLDDRPTRLGSPGVPMYGYNVQLLNEVTGEPCGVNEKGMLVVEGPLPPGCIQTIWGDDGRFVKTYWSLFSRPVYATFDWGIRDADGYHFILGRTDDVINVAGHRLGTREIEESISSHPGVAEVAVVGVKDALKGQVAVAFVIPKESDSLEDRDVAHSQEKAIMALVDSQIGNFGRPAHVWFVSQLPKTRSGEMLRRTIQAICEGRDPGDLTTIDDPASLDQIRQAMEE

SEQ ID NO: 42 - protein, 632 aa, Ralstonia solanacearum
MPMSAAYRALYQRSIDDPAGFWGEQAQRIDWQTPYGAVLDDSRLPFARWFVGGRTSLCHNAVDRHLATRGEQTALVYVSTETGIEAAYTYRALHREVNRMAACLQALGVRRGDRVLIYLPMIPEAAFAMLACARIGAIHSVVFGGFASNSLATRIDDAAPRVIVSADAGSRAGKVVEYKPLLDAAIDLAAHKPAHVLLVDRGLALMQHRAHDVDYATLARQHAHADVPCEWMESNEPSYILYTSGTTGKPKGVQRDTGGYAVALAASMPLIFGAQAGDTMFTASDVGWVVGHSYIVYAPLLAGLTTVMYEGTPVRPDGAVWWRIVEQYRVNVMFTAPTAIRVLKRQDPALLHRHDLSSLRRLFLAGEPLDEPTARWIGDALGKPIVDNYWQTETGWPMLAIPQGVEPSTPKLGSPGFPVYGYRLDILDEATGQPCAPGEKGLLAVAAPLPPGCMTTVWGDDARFLRTYWSAFPGRPVYSSFDWGVRDDEGYITILGRTDDVINVAGHRLGTREIEESLSSHPAIAEVAVVGVADPLKGQVAMGFAIVRDAARIAEPAGRLALEGELMRTVEGQLGAVARPSRVFFVNALPKTRSGKLLRRAMQAVAEGRDPGDLTTIEDPTALAQVRTAMQA

SEQ ID NO: 43 - protein, 139 aa, Acinetobacter baylyi
MLDAHISPEGTLSLQTIAMPADTNWSGDVFGGWIVSQMDLAGAIHAERFSKGRCATISINQMTFLVPVKVGDVISCYTKILKVGNTSIQMQIEVWDSHDSSRPPKRVTEGVFTFVAVDVKGNKRTIAEDLKQQFLQHAS

SEQ ID NO: 44 - protein, 329 aa, Escherichia coli
MHITYDLPVAIDDIIEAKQRLAGRIYKTGMPRSNYFSERCKGEIFLKFENMQRTGSFKIRGAFNKLSSLTDAEKRKGVVACSAGNHAQGVSLSCAMLGIDGKVVMPKGAPKSKVAATCDYSAEVVLHGDNFNDTIAKVSEIVEMEGRIFIPPYDDPKVIAGQGTIGLEIMEDLYDVDNVIVPIGGGGLIAGIAVAIKSINPTIRVIGVQSENVHGMAASFHSGEITTHRTTGTLADGCDVSRPGNLTYEIVRELVDDIVLVSEDEIRNSMIALIQRNKVVTEGAGALACAALLSGKLDQYIQNRKTVSIISGGNIDLSRVSQITGFVDA

SEQ ID NO: 45 - protein, 514 aa, Escherichia coli [sequence omitted]

SEQ ID NO: 46 - protein, 360 aa, Saccharomyces cerevisiae
MSIVYNKTPLLRQFFPGKASAQFFLKYECLQPSGSFKSRGIGNLIMKSAIRIQKDGKRSPQVFASSGGNAGFAAATACQRLSLPCTVVVPTATKKRMVDKIRNTGAQVIVSGAYWKEADTFLKTNVMNKIDSQVIEPIYVHPFDNPDIWEGHSSMIDEIVQDLKSQHISVNKVKGIVCSVGGGGLYNGIIQGLERYGLADRIPIVGVETNGCHVFNTSLKIGQPVQFKKITSIATSLGTAVISNQTFEYARKYNTRSVVIEDKDVIETCLKYTHQFNMVIEPACGAALHLGYNTKILENALGSKLAADDIVIIIACGGSSNTIKDLEEALDSMRKKDTPVIEVADNFIFPEKNIVNLKSA

SEQ ID NO: 47 - protein, 764 aa, Escherichia coli
MKVDIDTSDKLYADAWLGFKGTDWKNEINVRDFIQHNYTPYEGDESFLAEATPATTELWEKVMEGIRIENATHAPVDFDTNIATTITAHDAGYINQPLEKIVGLQTDAPLKRALHPFGGINMIKSSFHAYGREMDSEFEYLFTDLRKTHNQGVFDVYSPDMLRCRKSGVLTGLPDGYGRGRIIGDYRRVALYGISYLVRERELQFADLQSRLEKGEDLEATIRLREELAEHRHALLQIQEMAAKYGFDISRPAQNAQEAVQWLYFAYLAAVKSQNGGAMSLGRTASFLDIYIERDFKAGVLNEQQAQELIDHFIMKIRMVRFLRTPEFDSLFSGDPIWATEVIGGMGLDGRTLVTKNSFRYLHTLHTMGPAPEPNLTILWSEELPIAFKKYAAQVSIVTSSLQYENDDLMRTDFNSDDYAIACCVSPMVIGKQMQFFGARANLAKTLLYAINGGVDEKLKIQVGPKTAPLMDDVLDYDKVMDSLDHFMDWLAVQYISALNIIHYMHDKYSYEASLMALHDRDVYRTMACGIAGLSVATDSLSAIKYARVKPIRDENGLAVDFEIDGEYPQYGNNDERVDSIACDLVERFMKKIKALPTYRNAVPTQSILTITSNVVYGQKTGNTPDGRRAGTPFAPGANPMHGRDRKGAVASLTSVAKLPFTYAKDGISYTFSIVPAALGKEDPVRKTNLVGLLDGYFHHEADVEGGQHLNVNVMNREMLLDAIEHPEKYPNLTIRVSGYAVRFNALTREQQQDVISRTFTQAL

SEQ ID NO: 48 - protein, 246 aa, Escherichia coli
MSVIGRIHSFESCGTVDGPGIRFITFFQGCLMRCLYCHNRDTWDTHGGKEVTVEDLMKEVVTYRHFMNASGGGVTASGGEAILQAEFVRDWFRACKKEGIHTCLDTNGFVRRYDPVIDELLEVTDLVMLDLKQMNDEIHQNLVGVSNHRTLEFAKYLANKNVKVWIRYVVVPGWSDDDDSAHRLGEFTRDMGNVEKIELLPYHELGKHKWVAMGEEYKLDGVKPPKKETMERVKGILEQYGHKVMF

SEQ ID NO: 49 - protein, 127 aa, Shigella flexneri
MITGIQITKAANDDLLNSFWLLDSEKGEARCIVAKAGYAEDEVVAVSKLGDIEYREVPVEVKPEVRVEGGQHLNVNVLRRETLEDAVKHPEKYPQLTIRVSGYAVRFNSLTPEQQRDVIARTFTESL

SEQ ID NO: 50 - protein, 538 aa, Actinobacillus succinogenes
MTDLNKLVKELNDLGLTDVKEIVYNPSYEQLFEEETKPGLEGFDKGTLTTLGAVAVDTGIFTGRSPKDKYIVCDETTKDTVWWNSEAAKNDNKPMTQETWKSLRELVAKQLSGKRLFVVEGYCGASEKHRIGVRMVTEVAWQAHFVKNMFIRPTDEELKNFKADFTVLNGAKCTNPNWKEQGLNSENFVAFNITEGIQLIGGTWYGGEMKKGMFSMMNYFLPLKGVASMHCSANVGKDGDVAIFFGLSGTGKTTLSTDPKRQLIGDDEHGWDESGVFNFEGGCYAKTINLSQENEPDIYGAIRRDALLENVVVRADGSVDFDDGSKTENTRVSYPIYHIDNIVRPVSKAGHATKVIFLTADAFGVLPPVSKLTPEQTEYYFLSGFTAKLAGTERGVTEPTPTFSACFGAAFLSLHPIQYADVLVERMKASGAEAYLVNTGWNGTGKRISIKDTRGIIDAILDGSIEKAEMGELPIFNLAIPKALPGVDPAILDPRDTYADKAQWQVKAEDLANRFVKNFVKYTANPEAAKLVGAGPKA

SEQ ID NO: 51 - DNA, 1643 bp, Actinobacillus succinogenes
tcgctagggatccatgactgatttgaataaattagtcaaggaattaaacgacttgggtttgactgatgttaaggaaattgtttacaacccatcctacgaacaattgttcgaagaagaaactaagcctggtttggaaggtttcgataagggtaccttaactactttgggtgctgttgctgttgatactggtatcttcactggtagatccccaaaggacaagtacattgtctgtgatgaaaccactaaggataccgtttggtggaactctgaagctgctaagaacgataacaagccaatgactcaagaaacttggaagtccttgagagagttagttgctaagcaattgtctggtaagagattgtttgtcgttgaaggttactgtggtgcttctgaaaagcatagaattggtgtcagaatggttaccgaagttgcttggcaagcccacttcgttaagaacatgttcattcgtcctactgatgaagaattgaaaaacttcaaggctgacttcaccgtcttaaatggtgccaagtgtactaacccaaactggaaggaacaaggtttgaactccgaaaatttcgttgctttcaacatcaccgaaggtatccaattaattggtggtacttggtacggtggtgaaatgaagaagggtatgttttctatgatgaactattttttaccattaaagggtgttgcctccatgcattgttccgccaatgtcggtaaggatggtgatgtcgctattttcttcggtttgtctggtactggtaagaccactttgtctaccgacccaaagcgtcaattgatcggtgatgacgaacacggttgggacgaatctggtgtctttaacttcgaaggtggttgttacgctaaaaccatcaacttatctcaagaaaatgaaccagatatttatggtgctatcagaagagacgctttgttggaaaatgttgttgttagagccgatggttctgtcgatttcgatgacggttccaaaactgaaaacactagagtttcttacccaatttaccatattgacaacattgttagaccagtctccaaggctggtcatgccaccaaggttatcttcttgaccgccgatgctttcggtgttttaccaccagtttctaagttgaccccagaacaaaccgaatactacttcttgtctggtttcaccgctaagttggctggtaccgaaagaggtgttaccgaaccaactccaaccttttccgcttgtttcggtgctgccttcttgtctttgcacccaattcaatacgccgacgtcttggtcgaaagaatgaaggcttccggtgctgaagcctacttagtcaataccggttggaacggtactggtaaaagaatctccattaaggatactagaggtattattgatgctatcttggacggttctattgaaaaggccgaaatgggtgaattgcctatttttaacttggccatccctaaggctttgcctggtgttgacccagctattttggacccaagagatacttacgctgataaagctcaatggcaagtcaaggctgaggatttggctaacagattcgttaagaatttcgttaaatacactgccaatccagaagccgccaagttggtcggtgctggtccaaaggcctaactcgaggctcgac

SEQ ID NO: 52 - protein, 466 aa, Escherichia coli
MPHSYDYDAIVIGSGPGGEGAAMGLVKQGARVAVIERYQNVGGGCTHWGTIPSKALRHAVSRIIEFNQNPLYSDHSRLLRSSFADILNHADNVINQQTRMRQGFYERNHCEILQGNARFVDEHTLALDCPDGSVETLTAEKFVIACGSRPYHPTDVDFTHPRIYDSDSILSMHHEPRHVLIYGAGVIGCEYASIFRGMDVKVDLINTRDRLLAFLDQEMSDSLSYHFWNSGVVIRHNEEYEKIEGCDDGVIMHLKSGKKLKADCLLYANGRTGNTDSLALQNIGLETDSRGQLKVNSMYQTAQPHVYAVGDVIGYPSLASAAYDQGRIAAQALVKGEATAHLIEDIPTGIYTIPEISSVGKTEQQLTAMKVPYEVGRAQFKHLARAQIVGMNVGTLKILFHRETKEILGIHCFGERAAEIIHIGQAIMEQKGGGNTIEYFVNTTFNYPTMAEAYRVAALNGLNRLF

SEQ ID NO: 53 - protein, 464 aa, Azobacter vinelandii
MAVYNYDVVVIGTGPAGEGAAMNAVKAGRKVAVVDDRPQVGGNCTHLGTIPSKALRHSVRQIMQYNNNPLFRQIGEPRWFSFADVLKSAEQVIAKQVSSRTGYYARNRIDTFFGTASFCDEHTIEVVHLNGMVETLVAKQFVIATGSRPYRPADVDFTHPRIYDSDTILSLGHTPRRLIIYGAGVIGCEYASIFSGLGVLVDLIDNRDQLLSFLDDEISDSLSYHLRNNNVLIRHNEEYERVEGLDNGVILHLKSGKKIKADAFLWSNGRTGNTDKLGLENIGLKANGRGQIQVDEHYRTEVSNIYAAGDVIGWPSLASAAYDQGRSAAGSITENDSWRFVDDVPTGIYTIPEISSVGKTERELTQAKVPYEVGKAFFKGMARAQIAVEKAGMLKILFHRETLEILGVHCFGYQASEIVHIGQAIMNQKGEANTLKYFINTTFNYPTMAEAYRVAAYDGLNRLF

SEQ ID NO: 54 - DNA, 1401 bp, Escherichia coli
atgccacactcttacgattacgacgctattgttatcggttccggtccaggtggtgaaggtgctgctatgggtttggttaagcaaggtgctagagttgctgttattgaaagataccaaaacgtcggtggtggttgtactcactggggtaccattccatctaaagctttgagacatgctgtctccagaatcatcgaatttaaccaaaacccattgtactctgaccattctagattgttaagatcctccttcgccgacattttgaaccatgctgacaacgttattaaccaacaaactagaatgagacaaggtttctacgaaagaaatcattgtgaaatcttgcaaggtaatgctagattcgttgacgagcacaccttagccttagactgtccagatggttctgttgaaactttaactgctgagaaattcgttattgcttgtggttcccgtccataccacccaaccgatgttgacttcactcatccaagaatctacgactctgactctattttgtctatgcaccacgagccaagacatgttttgatctacggtgctggtgttatcggttgcgaatacgcttctatcttcagaggtatggatgttaaggttgacttgattaacactagagacagattgttagctttcttggaccaagaaatgtctgattccttgtcttaccacttctggaactctggtgtcgttattcgtcacaatgaagaatacgaaaagatcgagggttgtgacgatggtgttatcatgcacttgaaatccggtaagaagttaaaggctgattgtttgttgtacgctaatggtagaaccggtaataccgattctttggccttgcaaaatattggtttggagactgattctagaggtcaattgaaagtcaactccatgtatcaaactgctcaaccacatgtttacgctgttggtgatgtcattggttacccatccttggcttctgccgcttacgatcaaggtagaatcgctgctcaagccttggtcaagggtgaagctaccgctcacttgattgaagatattccaaccggtatctacactatcccagaaatctcttccgttggtaagactgaacaacaattgactgctatgaaggttccatatgaagtcggtcgtgcccaattcaagcatttggctagagcccaaatcgttggtatgaacgttggtactttgaagattttgtttcatagagagactaaggaaattttgggtattcattgtttcggtgaaagagctgctgaaattattcacattggtcaagctattatggaacaaaaaggtggtggtaacaccatcgaatatttcgttaacactacctttaactacccaactatggctgaagcttacagagtcgccgctttaaatggtttgaacagattgttctaa

SEQ ID NO: 55 - DNA, 1395 bp, Azobacter vinelandii
atggccgtttacaactatgatgtcgttgtcatcggtaccggtccagccggtgaaggtgccgccatgaacgccgttaaggccggtagaaaggttgctgttgttgacgatagaccacaagtcggtggtaactgtacccacttaggtaccattccttctaaagccttaagacactctgttagacaaattatgcaatacaacaacaacccattattcagacaaatcggtgaaccaagatggttttcttttgctgatgtcttaaagtccgctgaacaagttattgccaagcaagtctcctctagaactggttactacgctcgtaacagaattgatactttcttcggtactgcctccttctgtgatgaacacactattgaagtcgtccacttgaacggtatggtcgaaactttggttgccaagcaattcgtcatcgctactggttctagaccatacagaccagctgacgttgatttcacccaccctcgtatctatgattccgatactatcttgtccttgggtcataccccaagaagattgatcatctacggtgctggtgttattggttgtgaatacgcctctatcttttctggtttgggtgttttggttgatttgatcgacaaccgtgatcaattgttgtccttcttagatgacgagatctctgactctttgtcttaccacttgagaaacaacaacgtcttgattagacataacgaagaatacgaaagagttgaaggtttggataacggtgttatcttacacttgaagtctggtaagaagattaaggccgacgcctttttgtggtctaatggtagaaccggtaatactgataagttgggtttagaaaacattggtttgaaggccaacggtagaggtcaaattcaagttgacgagcactacagaactgaagtttccaacatttacgccgccggtgacgttattggttggccttccttggcttccgccgcctacgaccaaggtcgttctgctgctggttctatcactgaaaatgattcctggagattcgttgacgatgttccaaccggtatctacactatcccagaaatttcctctgttggtaagaccgaacgtgaattgactcaagctaaggtcccatacgaggttggtaaggccttttttaagggtatggctagagcccaaatcgccgtcgaaaaagctggtatgttgaagattttgttccatagagagactttggaaattttgggtgttcactgttttggttatcaagcctctgaaatcgtccacattggtcaagctatcatgaatcaaaagggtgaggctaacactttgaagtacttcatcaacaccactttcaactatccaactatggctgaagcctacagagttgctgcttacgacggtttgaacagattgttttaa

SEQ ID NO: 56 - protein, 1174 aa, Escherichia coli
MITIDGNGAVASVAFRTSEVIAIYPITPSSTMAEQADAWAGNGLKNVWGDTPRVVEMQSEAGAIATVHGALQTGALSTSFTSSQGLLLMIPTLYKLAGELTPFVLHVAARTVATHALSIFGDHSDVMAVRQTGCAMLCAANVQEAQDFALISQIATLKSRVPFIHFFDGFRTSHEINKIVPLADDTILDLMPQVEIDAHRARALNPEHPVIRGTSANPDTYFQSREATNPWYNAVYDHVEQAMNDFSAATGRQYQPFEYYGHPQAERVIILMGSAIGTCEEVVDELLTRGEKVGVLKVRLYRPFSAKHLLQALPGSVRSVAVLDRTKEPGAQAEPLYLDVMTALAEAFNNGERETLPRVIGGRYGLSSKEFGPDCVLAVFAELNAAKPKARFTVGIYDDVTNLSLPLPENTLPNSAKLEALFYGLGSDGSVSATKNNIKIIGNSTPWYAQGYFVYDSKKAGGLTVSHLRVSEQPIRSAYLISQADFVGCHQLQFIDKYQMAERLKPGGIFLLNTPYSADEVWSRLPQEVQAVLNQKKARFYVINAAKIARECGLAARINTVMQMAFFHLTQILPGDSALAELQGAIAKSYSSKGQDLVERNWQALALARESVEEVPLQPVNPHSANRPPVVSDAAPDFVKTVTAAMLAGLGDALPVSALPPDGTWPMGTTRWEKRNIAEEIPIWKEELCTQCNHCVAACPHSAIRAKVVPPEAMENAPASLHSLDVKSRDMRGQKYVLQVAPEDCTGCNLCVEVCPAKDRQNPEIKAINMMSRLEHVEEEKINYDFFLNLPEIDRSKLERIDIRTSQLITPLFEYSGACSGCGETPYIKLLTQLYGDRMLIANATGCSSIYGGNLPSTPYTTDANGRGPAWANSLFEDNAEFGLGFRLTVDQHRVRVLRLLDQFADKIPAELLTALKSDATPEVRREQVAALRQQLNDVAEAHELLRDADALVEKSIWLIGGDGWAYDIGFGGLDHVLSLTENVNILVLDTQCYSNTGGQASKATPLGAVTKFGEHGKRKARKDLGVSMMMYGHVYVAQISLGAQLNQTVKAIQEAEAYPGPSLIIAYSPCEEHGYDLALSHDQMRQLTATGFWPLYRFDPRRADEGKLPLALDSRPPSEAPEETLLHEQRFRRLNSQQPEVAEQLWKDAAADLQKRYDFLAQMAGKAEKSNTD

SEQ ID NO: 57 - protein, 1803 aa, Euglena gracilis
MKQSVRPIISNVLRKEVALYSTIIGQDKGKEPTGRTYTSGPKPASHIEVPHHVTVPATDRTPNPDAQFFQSVDGSQATSHVAYALSDTAFIYPITPSSVMGELADVWMAQGRKNAFGQVVDVREMQSEAGAAGALHGALAAGAIATTFTASQGLLLMIPNMYKIAGELMPSVIHVAARELAGHALSIFGGHADVMAVRQTGWAMLCSHTVQQSHDMALISHVATLKSSIPFVHFFDGFRTSHEVNKIKMLPYAELKKLVPPGTMEQHWARSLNPMHPTIRGTNQSADIYFQNMESANQYYTDLAEVVQETMDEVAPYIGRHYKIFEYVGAPDAEEVTVLMGSGATTVNEAVDLLVKRGKKVGAVLVHLYRPWSTKAFEKVLPKTVKRIAALDRCKEVTALGEPLYLDVSATLNLFPERQNVKVIGGRYGLGSKDFIPEHALAIYANLASENPIQRFTVGITDDVTGTSVPFVNERVDTLPEGTRQCVFWGIGSDGTVGANRSAVRIIGDNSDLMVQAYFQFDAFKSGGVTSSHLRFGPKPITAQYLVTNADYIACHFQEYVKRFDMLDAIREGGTFVLNSRWTTEDMEKEIPADFRRKLAQKKVRFYNVDARKICDSFGLGKRINMLMQACFFKLSGVLPLAEAQRLLNESIVHEYGKKGGKVVEMNQAVVNAVFAGDLPQEVQVPAAWANAVDTSTRTPTGIEFVDKIMRPLMDFKGDQLPVSVMTPGGTFPVGTTQYAKRAIAAFIPQWIPANCTQCNYCSYVCPHATIRPFVLTDQEVQLAPESFVTRKAKGDYQGMNFRIQVAPEDCTGCQVCVETCPDDALEMTDAFTATPVQRTNWEFAIKVPNRGTMTDRYSLKGSQFQQPLLEFSGACEGCGETPYVKLLTQLFGERTVIANATGCSSIWGGTAGLAPYTTNAKGQGPAWGNSLFEDNAEFGFGIAVANAQKRSRVRDCILQAVEKKVADEGLTTLLAQWLQDWNTGDKTLKYQDQIIAGLAQQRSKDPLLEQIYGMKDMLPNISQWIIGGDGWANDIGFGGLDHVLASGQNLNVLVLDTEMYSNTGGQASKSTHMASVAKFALGGKRTNKKNLTEMAMSYGNVYVATVSHGNMAQCVKAFVEAESYDGPSLIVGYAPCIEHGLRAGMARMVQESEAAIATGYWPLYRFDPRLATEGKNPFQLDSKRIKGNLQEYLDRQNRYVNLKKNNPKGADLLKSQMADNITARFNRYRRMLEGPNTKAAAPSGNHVTILYGSETGNSEGLAKELATDFERREYSVAVQALDDIDVADLENMGFVVIAVSTCGQGQFPRNSQLFWRELQRDKPEGWLKNLKYTVFGLGDSTYYFYCHTAKQIDARLAALGAQRVVPIGFGDDGDEDMFHTGFNNWIPSVWNELKTKTPEEALFTPSIAVQLTPNATPQDFHFAKSTPVLSITGAERITPADHTRNFVTIRWKTDLSYQVGDSLGVFPENTRSVVEEFLQYYGLNPKDVITIENKGSRELPHCMAVGDLFTKVLDILGKPNNRFYKTLSYFAVDKAEKERLLKIAEMGPEYSNILSETYHYADIFHMFPSARPTLQYLIEMIPNIKPRYYSISSAPIHTPGEVHSLVLIDTWITLSGKHRTGLTCTMLEHLQAGQVVDGCIHPTAMEFPDHEKPVVMCAMGSGLAPFVAFLRDGSTLRKQGKKTGNMALYFGNRYEKTEFLMKEELKGHINDGLLTLRCAFSRDDPKKKVYVQDLIKMDEKMMYDYLVVQKGSMYCCGSRSFIKPVQESLKHCFMKAGGLTAEQAENEVIDMFTTGRYNIEAW

SEQ ID NO: 58 - protein, 398 aa, Pseudomonas putida
MHGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 59 - protein, 324 aa, Zymomonas mobilis
MEIVAIDIGGTHARFSIAEVSNGRVLSLGEETTFKTAEHASLQLAWERFGEKLGRPLPRAAAIAWAGPVHGEVLKLTNNPWVLRPATLNEKLDIDTHVLINDFGAVAHAVAHMDSSYLDHICGPDEALPSDGVITILGPGTGLGVAHLLRTEGRYFVIETEGGHIDFAPLDRLEDKILARLRERFRRVSIERIISGPGLGNIYEALAAIEGVPFSLLDDIKLWQMALEGKDNLAEAALDRFCLSLGAIAGDLALAQGATSVVIGGGVGLRIASHLPESGFRQRFVSKGRFERVMSKIPVKLITYPQPGLLGAAAAYANKYSEVE

SEQ ID NO: 60 - protein, 314 aa, Thermotoga maritima
MKLIGVDLGGTTFSVGLVSEDGKILKKVTRDTLVENGKEDVIRRIAETILEVSDGEEAPYVGIGSPGSIDRENGIVRFSPNFPDWHNVPLTDELAKRTGKKVFLENDANAFVLGEKWFGAGRGHDHIVALTLGTGIGGGVVTHGYLLTGRDGIGAELGHVVVEPNGPMCNCGTRGCLEAVASATAIRRFLREGYKKYHSSLVYKLAGSPEKADAKHLFDAARQGDRFALMIRDRVVDALARAVAGYIHIFNPEIVIIGGGISRAGEILFGPLREKVVDYIMPSFVGTYEVVASPLVEDAGILGAASIIKERIGG

SEQ ID NO: 61 - protein, 332 aa, Lactobacillus plantarum
MKIIAYAVRDDERPFFDTWMKENPDVEVKLVPELLTEDNVDLAKGFDGADVYQQKDYTAEVLNKLADEGVKNISLRNVGVDNLDVPTVKARGLNISNVPAYSPNAIAELSVTQLMQLLRQTPLFNKKLAKQDFRWAPDIAKELNTMTVGVIGTGRIGRAAIDIFKGFGAKVIGYDVYRNAELEKEGMYVDTLDELYAQADVITLHVPALKDNYHMLNADAFSKMKDGAYILNFARGTLIDSEDLIKALDSGKVAGAALDTYEYETKIFNKDLEGQTIDDKVFMNLFNRDNVLITPHTAFYTETAVHNMVHVSMNSNKQFIETGKADTQVKFD

SEQ ID NO: 62 - protein, 329 aa, Escherichia coli
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 63 - protein, 326 aa, Lactobacillus caseii
MASITDKDHQKVILVGDGAVGSSYAYAMVLQGIAQEIGIVDIFKDKTKGDAIDLSNALPFTSPKKIYSAEYSDAKDADLVVITAGAPQKPGETRLDLVNKNLKILKSIVDPIVDSGFNGIFLVAANPVDILTYATWKLSGFPKNRVVGSGTSLDTARFRQSIAEMVNVDARSVHAYIMGEHGDTEFPVWSHANIGGVTIAEWVKAHPEIKEDKLVKMFEDVRDAAYEIIKLKGATFYGIATALARISKAILNDENAVLPLSVYMDGQYGLNDIYIGTPAVINRNGIQNILEIPLTDHEEESMQKSASQLKKVLTDAFAKNDIETRQ

SEQ ID NO: 64 - protein, 517 aa, Megasphaera elsdenii
MRKVEIITAEQAAQLVKDNDTITSIGFVSSAHPEALTKALEKRFLDTNTPQNLTYIYAGSQGKRDGRAAEHLAHTGLLKRAIIGHWQTVPAIGKLAVENKIEAYNFSQGTLVHWFRALAGHKLGVFTDIGLETFLDPRQLGGKLNDVTKEDLVKLIEVDGHEQLFYPTFPVNVAFLRGTYADESGNITMDEEIGPFESTSVAQAVHNCGGKVVVQVKDVVAHGSLDPRMVKIPGIYVDYVVVAAPEDHQQTYDCEYDPSLSGEHRAPEGATDAALPMSAKKIIGRRGALELTENAVVNLGVGAPEYVASVAGEEGIADTITLTVEGGAIGGVPQGGARFGSSRNADAIIDHTYQFDFYDGGGLDIAYLGLAQCDGSGNINVSKFGTNVAGCGGFPNISQQTPNVYFCGTFTAGGLKIAVEDGKVKILQEGKAKKFIKAVDQITFNGSYAARNGKHVLYITERCVFELTKEGLKLIEVAPGIDIEKDILAHMDFKPIIDNPKLMDARLFQDGPMGLKK

SEQ ID NO: 65 - protein, 524 aa, Clostridium propionicum
MRKVPIITADEAAKLIKDGDTVTTSGFVGNAIPEALDRAVEKRFLETGEPKNITYVYCGSQGNRDGRGAEHFAHEGLLKRYIAGHWATVPALGKMAMENKMEAYNVSQGALCHLFRDIASHKPGVFTKVGIGTFIDPRNGGGKVNDITKEDIVELVEIKGQEYLFYPAFPIHVALIRGTYADESGNITFEKEVAPLEGTSVCQAVKNSGGIVVVQVERVVKAGTLDPRHVKVPGIYVDYVVVADPEDHQQSLDCEYDPALSGEHRRPEVVGEPLPLSAKKVIGRRGAIELEKDVAVNLGVGAPEYVASVADEEGIVDFMTLTAESGAIGGVPAGGVRFGASYNADALIDQGYQFDYYDGGGLDLCYLGLAECDEKGNINVSRFGPRIAGCGGFINITQNTPKVFFCGTFTAGGLKVKIEDGKVIIVQEGKQKKFLKAVEQITFNGDVALANKQQVTYITERCVFLLKEDGLHLSEIAPGIDLQTQILDVMDFAPIIDRDANGQIKLMDAALFAEGLMGLKEMKS

SEQ ID NO: 66 - protein, 531 aa, Escherichia coli
MKPVKPPRINGRVPVLSAQEAVNYIPDEATLCVLGAGGGILEATTLITALADKYKQTQTPRNLSIISPTGLGDRADRGISPLAQEGLVKWALCGHWGQSPRISELAEQNKIIAYNYPQGVLTQTLRAAAAHQPGIISDIGIGTFVDPRQQGGKLNEVTKEDLIKLVEFDNKEYLYYKAIAPDIAFIRATTCDSEGYATFEDEVMYLDALVIAQAVHNNGGIVMMQVQKMVKKATLHPKSVRIPGYLVDIVVVDPDQTQLYGGAPVNRFISGDFTLDDSTKLSLPLNQRKLVARRALFEMRKGAVGNVGVGIADGIGLVAREEGCADDFILTVETGPIGGITSQGIAFGANVNTRAILDMTSQFDFYHGGGLDVCYLSFAEVDQHGNVGVHKFNGKIMGTGGFIDISATSKKIIFCGTLTAGSLKTEITDGKLNIVQEGRVKKFIRELPEITFSGKIALERGLDVRYITERAVFTLKEDGLHLIEIAPGVDLQKDILDKMDFTPVISPELKLMDERLFIDAAMGFVLPEAAH

SEQ ID NO: 67 - protein, 713 aa, Saccharomyces cerevisiae
MSPSAVQSSKLEEQSSEIDKLKAKMSQSASTAQQKKEHEYEHLTSVKIVPQRPISDRLQPAIATHYSPHLDGLQDYQRLHKESIEDPAKFFGSKATQFLNWSKPFDKVFIPDSKTGRPSFQNNAWFLNGQLNACYNCVDRHALKTPNKKAIIFEGDEPGQGYSITYKELLEEVCQVAQVLTYSMGVRKGDTVAVYMPMVPEAIITLLAISRIGAIHSVVFAGFSSNSLRDRINDGDSKVVITTDESNRGGKVIETKRIVDDALRETPGVRHVLVYRKTNNPSVAFHAPRDLDWATEKKKYKTYYPCTPVDSEDPLFLLYTSGSTGAPKGVQHSTAGYLLGALLTMRYTFDTHQEDVFFTAGDIGWITGHTYVVYGPLLYGCATLVFEGTPAYPNYSRYWDIIDEHKVTQFYVAPTALRLLKRAGDSYIENHSLKSLRCLGSVGEPIAAEVWEWYSEKIGKNEIPIVDTYWQTESGSHLVTPLAGGVTPMKPGSASFPFFGIDAVVLDPNTGEELNTSHAEGVLAVKAAWPSFARTIWKNHDRYLDTYLNPYPGYYFTGDGAAKDKDGYIWILGRVDDVVNVSGHRLSTAEIEAAIIEDPIVAECAVVGFNDDLTGQAVAAFVVLKNKSNWSTATDDELQDIKKHLVFTVRKDIGPFAAPKLIILVDDLPKTRSGKIMRRILRKILAGESDQLGDVSTLSNPGIVRHLIDSVKL

SEQ ID NO: 68 - protein, 652 aa, Escherichia coli
MSQIHKHTIPANIADRCLINPQQYEAMYQQSINVPDTFWGEQGKILDWIKPYQKVKNTSFAPGNVSIKWYEDGTLNLAANCLDRHLQENGDRTAIIWEGDDASQSKHISYKELHRDVCRFANTLLELGIKKGDVVAIYMPMVPEAAVAMLACARIGAVHSVIFGGFSPEAVAGRIIDSNSRLVITSDEGVRAGRSIPLKKNVDDALKNPNVTSVEHVVVLKRTGGKIDWQEGRDLWWHDLVEQASDQHQAEEMNAEDPLFILYTSGSTGKPKGVLHTTGGYLVYAALTFKYVFDYHPGDIYWCTADVGWVTGHSYLLYGPLACGATTLMFEGVPNWPTPARMAQVVDKHQVNILYTAPTAIRALMAEGDKAIEGTDRSSLRILGSVGEPINPEAWEWYWKKIGNEKCPVVDTWWQTETGGFMITPLPGATELKAGSATRPFFGVQPALVDNEGNPLEGATEGSLVITDSWPGQARTLFGDHERFEQTYFSTFKNMYFSGDGARRDEDGYYWITGRVDDVLNVSGHRLGTAEIESALVAHPKIAEAAVVGIPHNIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 69 - protein, 259 aa, Clostridium propionicum
MYTLGIDVGSASSKAVILKDGKDIVAAEVVQVGTGSSGPQRALDKAFEVSGLKKEDISYTVATGYGRFNFSDADKQISEISCHAKGIYFLVPTARTIIDIGGQDAKAIRLDDKGGIKQFFMNDKCAAGTGRFLEVMARVLETTLDEMAELDEQATDTAPISSTCTVFAESEVISQLSNGVSRNNIIKGVHLSVASRACGLAYRGGLEKDVVMTGGVAKNAGVVRAVAGVLKTDVIVAPNPQTTGALGAALYAYEAAQKK

SEQ ID NO: 70 - protein, 422 aa, Clostrodium propionicum
MSLTQGMKAKQLLAYFQGKADQDAREAKARGELVCWSASVAPPEFCVTMGIAMIYPETHAAGIGARKGAMDMLEVADRKGYNVDCCSYGRVNMGYMECLKEAAITGVKPEVLVNSPAADVPLPDLVITCNNICNTLLKWYENLAAELDIPCIVIDVPFNHTMPIPEYAKAYIADQFRNAISQLEVICGRPFDWKKFKEVKDQTQRSVYHWNRIAEMAKYKPSPLNGFDLFNYMALIVACRSLDYAEITFKAFADELEENLKAGIYAFKGAEKTRFQWEGIAVWPHLGHTFKSMKNLNSIMTGTAYPALWDLHYDANDESMHSMAEAYTRIYINTCLQNKVEVLLGIMEKGQVDGTVYHLNRSCKLMSFLNVETAEIIKEKNGLPYVSIDGDQTDPRVFSPAQFDTRVQALVEMMEANMAAAE

SEQ ID NO: 71 - protein, 374 aa, Clostridium propionicum
MSRVEAILSQLKDVAANPKKAMDDYKAETGKGAVGIMPIYSPEEMVHAAGYLPMGIWGAQGKTISKARTYLPAFACSVMQQVMELQCEGAYDDLSAVIFSVPCDTLKCLSQKWKGTSPVIVFTHPQNRGLEAANQFLVTEYELVKAQLESVLGVKISNAALENSIAIYNENRAVMREFVKVAADYPQVIDAVSRHAVFKARQFMLKEKHTALVKELIAEIKATPVQPWDGKKVVVTGILLEPNELLDIFNEFKIAIVDDDLAQESRQIRVDVLDGEGGPLYRMAKAWQQMYGCSLATDTKKGRGRMLINKTIQTGADAIVVAMMKFCDPEEWDYPVMYREFEEKGVKSLMIEVDQEVSSFEQIKTRLQSFVEML

SEQ ID NO: 72 - protein, 326 aa, Rhodobacter sphaeroides
MRAVLIEKSDDTQSVSVTELAEDQLPEGDVLVDVAYSTLNYKDALAITGKAPVVRRFPMVPGIDFTGTVAQSSHADFKPGDRVILNGWGVGEKHWGGLAERARVRGDWLVPLPAPLDLRQAAMIGTAGYTAMLCVLALERHGVVPGNGEIVVSGAAGGVGSVATTLLAAKGYEVAAVTGRASEAEYLRGLGAASVIDRNELTGKVRPLGQERWAGGIDVAGSTVLANMLSMMKYRGVVAACGLAAGMDLPASVAPFILRGMTLAGVDSVMCPKTDRLAAWARLASDLDPAKLEEMTTELPFSEVIETAPKFLDGTVRGRIVIPVTP

SEQ ID NO: 73 - protein, 379 aa, Clostridium kluyveri
MDFTLTNEQKFVEQMVSEFTENEVKPIAAEIDETERFPLETVEKFAKYGMMGMPFPVEYGGSGTDYLSYIIAVEGLAKSCTSSSTILSAHTSLCAAPIYDWGTEEQKQKYLVPLAKGEKLGAFGLTEPNAGTDAAGQQTTAVLEGDHYVLNGQKIFITNGAYADTFVIFAMTDRSKGTRGITAFIVEKDFPGFSIGKSEDKLGIRASSTTELIFENCIVPKENMLGKEGKGFTVAMHTLDGGRIGIAAQALGLAEGALAEALNYMKERKQFGKALYKFQGLAWMVAELDTKIEAVKQLVYKAAVNKQMGLPYSVEAARAKLAAATVAMETTTKVVQIFGGYGFTKDYPVERMMRDAKITEIYEGTSQVQKMVISANLFK

SEQ ID NO: 74 - protein, 351 aa, Bacillus cereus
MTEHVLFSVSENGVASITLNRPKALNSLSYDMLQPIGQKLKEWENDERIALIVLKGAGTKGFCAGGDIKTLYEARSNEAALQHAERFFEEEYEIDTYIYQYKKPIIACLDGIVMGGGVGLTNGAKYRIVTERTKWAMPEMNIGFFPDVGAAYFLNKAPGYAGRYVALTASILKASDVLFINAADYFIASDSLPNFLTELESVNWSKEDDVHTHLKEVIRTFATAPTLESELAPSLEEINSHFAFDTIEEIIHSLEKDQSSFSLKAKETLLSKSPISLKVTLKQFIDGQNKSVEECFATDLVLAKNFMRHEDFFEGVRSVVVDKDQNPNYKYKQLSDVSEEDVNRFFNLLNA

(19) United States
(12) Patent Application Publication
(10) Pub. No.: US 2016/0230198 A1
(43) Pub. Date: Aug. 11, 2016

(54) Methylmalonic acid compositions, biological methods for making same, and microorganisms for making same

(71) Applicant: GeneSys Consulting, LLC, Plymouth, MN (US)

(72) Inventor: Goutham N. Vemuri, Plymouth, MN (US)

(73) Assignee: GeneSys Consulting, LLC, Plymouth, MN (US)

(21) Appl. No.: 15/019,963

(22) Filed: Feb. 9, 2016

Related U.S. Application Data
(63) Continuation of application No. PCT/US16/17218, filed on Feb. 9, 2016.
(60) Provisional application No. 62/114,541, filed on Feb. 10, 2015.

(51) Int. Cl. C12P 7/46; C12N 9/00; C12N 9/16; C07C 55/08

(52) U.S. Cl. CPC C12P 7/46; C07C 55/08; C12N 9/93; C12N 9/16; C12Y 602/01017; C12Y 301/02017

(57) ABSTRACT

Microorganisms and methods are provided for biological synthesis of methylmalonic acid and derivatives thereof. Engineered microorganisms such as bacteria, yeast, and fungi are configured to produce or overproduce methylmalonic acid and/or derivatives thereof. Methods involve the use of such engineered microorganisms to produce methylmalonic acid and/or derivatives thereof from carbon sources. Methods may include production in a fermenter and optional purification of the product.

### CROSS-REFERENCE TO RELATED APPLICATIONS

This application is a continuation of International Application No. PCT/US16/17218, filed Feb. 9, 2016, which claims the benefit of U.S. Provisional Application Number 62/114541, filed Feb. 10, 2015, and entitled “Microorganisms for the Synthesis of Methylmalonic Acid and Derivatives.” Each of the above-identified applications are herein incorporated by reference in their entirety.

### FIELD

This disclosure generally relates to microbiology and biochemical technology. This disclosure also relates to non-natural microorganisms for producing biochemicals from carbon sources. And this disclosure relates to methods for biological synthesis of biochemicals such as methylmalonic acid and its derivatives, from carbon substrates.

### BACKGROUND

Methylmalonic acid (“MMA”) is used as an indicator of vitamin B deficiency. However, MMA is naturally produced in only very small quantities in cells in response to a deficiency in Vitamin B12 or metabolic aciduria through the citrate cycle or branched-chain amino acid (valine, leucine and isoleucine) degradation pathways.

FIG. 29, which can be found at http.//www.genome.jp/kegg-bin/show_pathwav?map00280, shows the reactions from the citrate cycle to methylmalonate as well as the degradation pathways of branched-chain amino acids (valine, leucine and isoleucine), which involve methylmalonate. FIG. 29 suggests that methylmalonate can be produced from methylmalonyl-CoA or methylmalonate semialdehyde suggesting it is by the action of a methylmalonyl-CoA hydrolase (EC 3.1.2.17) or methylmalonate semialdehyde (EC 1.2.1.3 or EC 1.2.3.1), respectively. What was once thought to be an EC 3.1.2.17 enzyme was later shown to be an EC 3.1.2.4 enzyme and EC 1.2.1.3 and EC 1.2.3.1 are generic dehydrogenases that act on a number of aldehydes and semialdehydes; any methylmalonic acid that was detected ultimately was attributed to the promiscuity of other coenzyme A hydrolases and dehydrogenases acting on methylmalonyl-CoA and methylmalonate semialdehyde. The actual genes for enzymes catalyzing these specific reactions for producing methylmalonic acid have not been identified and are not known to exist.

More specifically, Kovachy et al. (1983 and 1988) investigated the origin of biologically-derived methylmalonic acid in rats and claimed that a protein of molecular weight 35 kDa catalyzes the hydrolysis of (S)-methylmalonyl-CoA, but not (R)-methylmalonyl-CoA, into methylmalonate along with having promiscuous activity on malonyl-CoA, propionyl-CoA, acetyl-CoA and palmitoyl-CoA (Kovachy et al., Recognition, isolation, and characterization of rat liver D-methylmalonyl coenzyme A hydrolase, J Biol Chem, 1983, 258(18), 11415-21; Kovachy et al., D-methylmalonyl-CoA hydrolase, Methods in Enzymol, 1988, 166: 393-400). Indeed this gene is believed to be responsible for the production of methylmalonic acid in biological samples such as urine, in response to vitamin B12 deficiency (see e.g. Kwok T, Cheng G, Lai W K, Poon P, Woo J, Pang C P: Use of fasting urinary methylmalonic acid to screen for metabolic vitamin B12 deficiency in older persons. Nutrition 2004, 20(9):764-768) or metabolic aciduria (see e.g. Rosenberg L E, Lilljeqvist A C, Hsia Y E: Methylmalonic aciduria. An inborn error leading to metabolic acidosis, long-chain ketonuria and intermittent hyperglycinemia. The New England journal of medicine 1968, 278(24):1319-1322). However, the observations of Kovachy et al., 1983 and Kovachy et al., 1988 were due to the promiscuity of 3-hydroxyisobutyryl-CoA hydrolase, which was demonstrated to act on (S)-methylmalonyl-CoA as well (Shimomura, Y. et al. 3-hydroxyisobutyryl-CoA hydrolase. Methods in enzymology, 2000, 324, 229-240). The rat 3-hydroxyisobutyryl-CoA hydrolase catalyzed the hydrolysis of other CoA compounds such as 3-hydroxypropionyl-CoA, 3-hydroxybutyryl-CoA, acetoacetyl-CoA, isobutyryl-CoA, etc, although with much lower specificity (Shimomura, Y. et al. Purification and partial characterization of 3-hydroxyisobutyryl-coenzyme A hydrolase of rat liver. The J Biol Chem, 1994, 269, 14248-14253). The corresponding gene from yeast was modified to hydrolyze malonyl-CoA in WO2013134424. The size of the product of the gene that encodes for 3-hydroxyisobutyryl-coenzyme A is approximately 35 kDa, misleading Kovachy et al., (1983 and 1988) to wrongly believe that this gene product was methylmalonyl-CoA hydrolase.

US 2009/0186358 allegedly discloses the engineering of cells to up-regulate or down-regulate the genes or proteins of the valine, leucine and isoleucine degradation pathway to increase methylmalonate production. However, because the genes encoding for these enzymes are not known, and there are no methods disclosed for identifying the same, it was not possible to engineer cells to increase methylmalonate production from either methylmalonyl-CoA or methylmalonate semialdehyde.

US20100190224 allegedly describes the production of 3-hydroxyisobutyric acid from methylmalonyl-CoA and allegedly describes the use of an enzyme that can hydrolyze methylmalonyl-CoA. However, the sequence of the corresponding gene and the enzyme related to methylmalonyl-CoA hydrolase activity are not disclosed and are only hypothetical.

### SUMMARY

The present disclosure relates to engineered microorganisms configured to produce methylmalonic acid, biological methods of making methylmalonic acid using the said microorganisms, and methylmalonic acid compositions produced by the said biological methods.

In some embodiments, the engineered microorganism is configured to produce or overproduce a target chemical chosen from methylamlonic acid or its esters. The microorganism may be a bacteria, yeast, or filamentous fungus. In some embodiments, the microorganism is also engineered to secrete the target chemical. In some embodiments, the microorganism comprises at least one exogenous nucleic acid sequence encoding at least one polypeptide for converting a metabolic intermediate into a target chemical. In further embodiments, at least one polypeptide encodes for at least one enzyme capable of facilitating a step in a pathway for producing methylmalonic acid from methylmalonyl-CoA. In some embodiments, methylmalonyl-CoA is produced from propionyl-CoA and/or succinyl-CoA. In some embodiments, wherein the microorganism has a cytoplasm, the microorganism is further engineered to produce the target chemical in the cytoplasm.

In some embodiments, propionyl-CoA is produced from propanoate, by the reduction of acryloyl-CoA, by the oxidative decarboxylation of 2-oxobutanoate or the oxidation of odd-chain fatty acids. In some embodiments, 2-oxobutanoate is produced by the deamination of amino acids such as threonine or methionine. In some embodiments, acryloyl-CoA is produced from lactoyl-CoA or 3-hydroxypropanoyl-CoA.

In some embodiments, succinyl-CoA is produced from succinate or by the oxidative decarboxylation of α-ketoglutarate.

In some embodiments, the microorganism is engineered to increase the carbon flux to propionyl-CoA and/or succinyl-CoA.

In some embodiments, the methods involve using an engineered microorganism, such as described herein, to produce a target chemical chosen from methyl malonic acid and esters of methylmalonic acid. In some embodiments, the method also involves secreting the target chemical from the microorganism. In some embodiments, the target chemical is produced in a fermenter by the engineered microorganism, and the target chemical is optionally purified. In some embodiments, the method involves contacting an engineered microorganism with a carbon substrate wherein the microorganism is engineered to produce enzymes in a metabolic pathway (such as described herein) that produces methylmalonic acid and/or its esters from the carbon substrate. In further embodiments, the method involves culturing the microorganism under conditions whereby methylmalonic acid is produce and harvesting the methylmalonic acid. In some embodiments, the microorganism is further engineered to minimize competing metabolic pathways.

### BRIEF DESCRIPTION OF THE DRAWINGS

FIG. 1: Conversion of (S)-methylmalonyl-CoA into methylmalonate

FIG. 2: Metabolic pathways to produce methylmalonate via propionyl-CoA

FIG. 3: Metabolic pathways to produce methylmalonate via succinyl-CoA

FIG. 4: Plasmid map of pGC203

FIG. 5: Plasmid map of pGC314

FIG. 6: Methylmalonic acid production by engineered yeast

FIG. 7: Plasmid map of pGC406

FIG. 8: Plasmid map of pGC412

FIG. 9: Plasmid map of pGC432

FIG. 10: Plasmid map of pGC532

FIG. 11: Methylmalonic acid production by engineered bacteria

FIG. 12: Plasmid map of pGC588

FIG. 13: Plasmid map of pGC610

FIG. 14: Methylmalonyl-CoA hydrolase activity in engineered bacteria

FIG. 15: Plasmid map of pGC617

FIG. 16: Plasmid map of pGC618

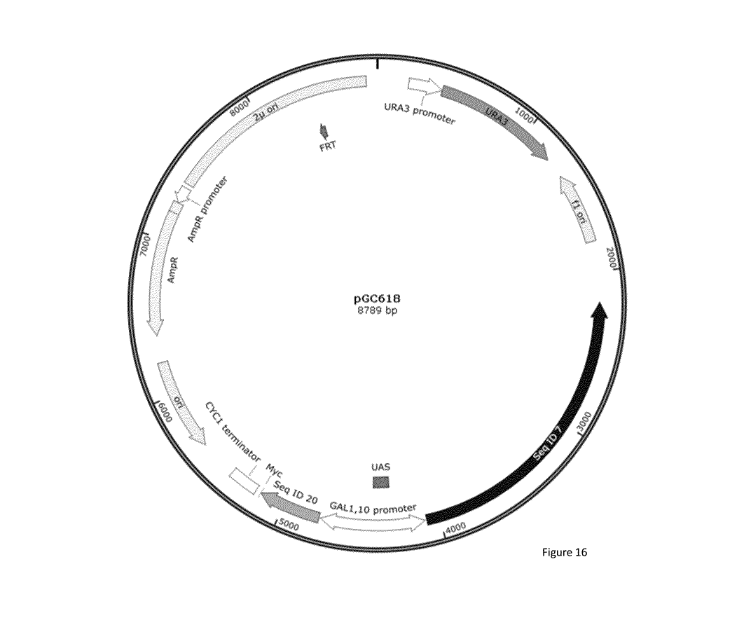

FIG. 17: Methylmalonyl-CoA hydrolase activity in engineered yeast

FIG. 18: Plasmid map of pGC711

FIG. 19: Plasmid map of pGC712

FIG. 20: Plasmid map of pGC713

FIG. 21: Plasmid map of pGC714

FIG. 22: Specificity of engineered methylmalonyl-CoA hydrolases to methylmalonyl-CoA

FIG. 23: Methylmalonic acid production by engineered methylmalonyl-CoA hydrolases

FIG. 24: Plasmid map of pGC756

FIG. 25: Plasmid map of pGC781

FIG. 26: Plasmid map of pGC782

FIG. 27: Schematic depicting the metabolic pathways to the derivatives of methylmalonic acid.

FIG. 28: Schematic of metabolic pathways from glutamate to methylmalonic acid.

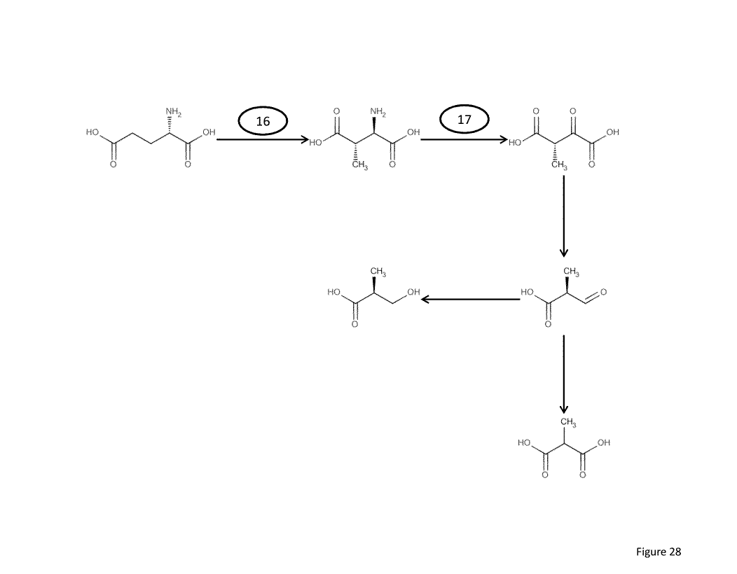

FIG. 29: KEGG screenshot of the metabolic pathways

### DESCRIPTION

The present disclosure relates to the design of non-natural microorganisms with an engineered metabolism to enable the production of biochemicals, such as industrial biochemicals, from carbon sources, including cheap carbon sources. More specifically, the engineered metabolic network facilitates the conversion of carbon substrates into methylmalonic acid and/or derivatives thereof. Carbon sources include, but not limited to, sugars such as glucose, fructose, sucrose, xylose and arabinose or their polymers, propanoate, fatty acids, glycerol, amino acids such as valine, leucine, and isoleucine, keto acids such as 2-oxobutanoic acid and pyruvate, and C1 substrates such as methane, carbon monoxide and carbon dioxide.

The present disclosure therefore provides means to engineer microorganisms with the capability to produce methylmalonic acid and/or esters thereof from carbon substrates such as those listed above by virtue of introducing nucleotide sequences encoding for one or more polypeptides that catalyze the enzymatic reactions in metabolic pathways that convert substrates to the desired products (“target chemicals”).

As used herein, the terms “polypeptide”, “peptide”, “protein” or “enzyme” are used interchangeably.

The sequences, including those naturally occurring as well as engineered, disclosed herein are intended to endow the microorganism with the ability to catalyze the desired reaction. It is understood that other enzymes that can catalyze the desired reactions are also within the scope of the disclosure. The skilled person will readily recognize that such enzymes may have a sequence identity of at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% and will understand that they are not excluded from this disclosure.

As used herein, the acid and its conjugated base are used interchangeably, and refer to the molecule in context. For example, “methylmalonic acid” and “methylmalonate” refer to the same chemical, unless specifically distinguished.

As used herein, an engineered microorganism is one that is genetically modified from its corresponding wild-type. For example, the genetic modification could be one or more of: (i) introduction of exogenous nucleic acid sequences; (ii) introduction of additional copies of endogenous sequences; (iii) deletion of endogenous sequences and (iv) alteration of promoter or terminator sequences.

In some embodiments, wherein the microorganism has a cytoplasm, the microorganism may be further engineered to produce at least a portion, or at least a majority, or at least almost entirely, the target chemical in the cytoplasm. Identification and deletion of mitochondrial signal sequence to direct proteins into the cytosol is well-documented in the art (Strand M K, Stuart G R, Longley M J, Graziewicz M A, Dominick O C, Copeland W C (2003) POS5 gene of Saccharomyces cerevisiae encodes a mitochondrial NADH kinase required for stability of mitochondrial DNA. Eukaryot Cell 2:809-820; http://www.cbs.dtu.dk/services/; http://ihg.gsf.de/ihg/mitoprot.html).

### Metabolic Pathways for Methylmalonic Acid Production

Those skilled in the art will understand that the herein disclosed pathways are described in relation to, but are not limited to, species-specific genes and proteins and that the invention encompasses homologs and orthologs of such gene and protein sequences. Homolog and ortholog sequences possess a relatively high degree of sequence identity (i.e. from about 65% to about 100% sequence identity) when aligned using methods known in the art. Algorithms well known to those skilled in the art, such as Align, BLAST, Clustal W and others compare and determine a raw sequence similarity or identity. A computer comparison of two or more sequences can, if desired, also be optimized visually by those skilled in the art. Related gene products or proteins can be expected to have a high similarity, for example, 65% to 100% sequence identity. In some embodiments, useful polypeptide sequences have at least 65%, at least 75%, at least 85%, at least 90%, or at least 95% or at least 99% identity to the amino acid sequence of the reference enzyme of interest.

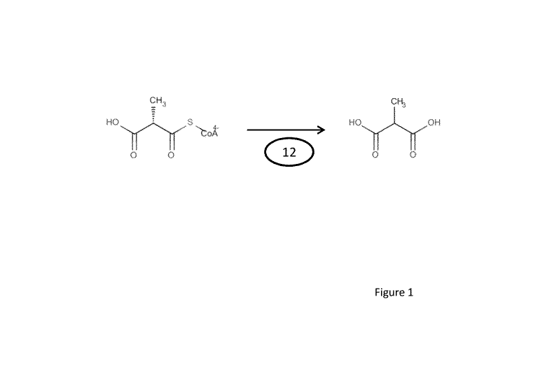

In some embodiments, a metabolic pathway is provided for the production of methylmalonic acid from (S)-methylmalonyl-CoA as illustrated in FIG. 1 by the action of methylmalonyl-CoA hydrolase (EC 3.1.2.17). Exemplary proteins that catalyze this kind of reaction are illustrated in Table 1

TABLE 1
Exemplary CoA hydrolase reactions and the UniProt IDs of some enzymes with such CoA hydrolase activity

| Enzyme name/ | | |
|---|---|---|
| UniProt ID | EC # | Reaction |
| Desired reaction Methylmalonyl-CoA hydrolase | 3.1.2.17 | 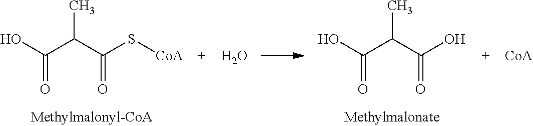 |
| Acetyl-CoA hydrolase UniProt ID: Q754Q2 P83773 Q6FPF3 Q6BKW1 | 3.1.2.1 | 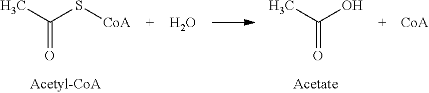 |
| Q54K91 | | |
| Q6CNR2 | | |
| P15937 | | |
| Q9UUJ9 | | |
| Q6C3Z9 | | |
| P32316 | | |
| Q8WYK0 | | |
| Q9DBK0 | | |
| Q99NB7 | | |
| 3-hydroxyisobutyryl CoA hydrolase UniProt ID: Q9LKJ1 Q1PEY5 Q6NMB0 Q2HJ73 Q5ZJ60 | 3.1.2.4 | 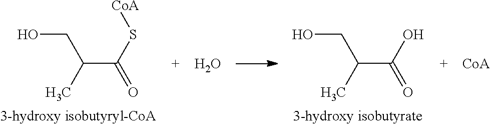 |
| Q58EB4 | | |
| Q55GS6 | | |
| Q6NVY1 | | |
| Q8QZS1 | | |
| Q5XIE6 | | |
| O74802 | | |
| A2VDC2 | | |
| Q28FR6 | | |
| P28817 | | |
| Acetoacetyl-CoA hydrolase UniProt ID: P33752 P23673 | 3.1.2.11 | 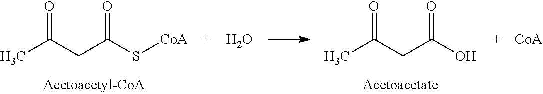 |
| Succinyl-CoA hydrolase UniProt ID: ESZKR7 | 3.1.2.3 | 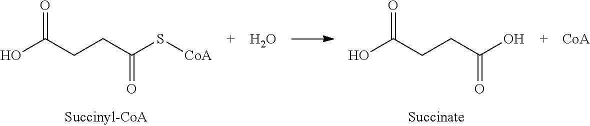 |
| Formyl-CoA hydrolase | 3.1.2.10 | 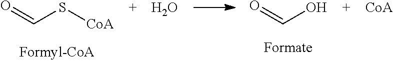 |

Despite the publications of Kovachy et al., (1983 and 1988), genes for Methylmalonyl-CoA hydrolase (E.C. 3.1.2.17) have not been identified and are not known to exist. To the inventor's knowledge, this specification discloses such agene for the first time. In some embodiments, the catalytic promiscuity of some enzymes, such as enzymes listed in Table 1, may be combined with protein engineering to modify the protein such that it may be exploited in novel metabolic pathways and biosynthesis applications. In some embodiments, and as shown in Example 5, the catalytic promiscuity of 3-hydroxyisobutyryl CoA hydrolase is exploited to modify its function using protein engineering to produce an enzyme that is more consistent with a Methylmalonyl-CoA hydrolase.

For example, in some embodiments, the non-natural microorganism contains an engineered gene that encodes for a modified (S)-methylmalonyl-CoA hydrolase with higher specificity for (S)-methylmalonyl-CoA than the naturally occurring enzyme. Based on the crystal structure (PDB ID: 3BPT) of the human 3-hydroxyisobutyryl-CoA hydrolase, the mechanism of action of the enzyme was hypothesized which was validated by a series of site-directed mutagenesis (Rouhier, M. F., Characterization of YDR036C from Saccharomyces cerevisiae, PhD Thesis, 2011, Miami University, Oxford, Ohio, USA). The amino acids that were deemed important for the activity of 3-hydroxyisobutyryl-CoA hydrolase in yeast are Glutamate-124 (interacts with the β-hyroxyl group of 3-hydroxyisobutyric acid), Phenylalanine-177 (responsible for the substrate specificity of the enzyme) and Serine-328 (subject to post-translational regulation via phosphorylation). In the examples below, the present disclosure demonstrates that these amino acids are also relevant increasing the substrate-specificity for (S)-methylmalonyl-CoA. In some embodiments, the mitochondrial signal sequence is removed in the engineered gene to allow for cytosolic localization, as described in the examples. In some embodiments, the non-natural microorganism contains an engineered gene that encodes for a modified (S)-methylmalonyl-CoA hydrolase with higher specificity for (S)-methylmalonyl-CoA than the naturally occurring enzyme and comprising an amino acid sequence having at least 65%, at least 70%, at least 75%, at least 80%, at least 85%, at least 90%, at least 95%, at least 96%, at least 97%, at least 98%, at least 99% identity to a sequence selected from SEQ ID 10, 32, 34, 36 or 38. In some embodiments, the amino acids at the positions Glu-124, Phe-177 and Ser-328 with respect to the sequence of the EHD3 gene from S. cerevisiae (UniProt ID: P28817) are altered in the (S)-methylmalonyl-CoA hydrolase. In some embodiments, the engineered enzyme also has (R)-methylmalonyl-CoA hydrolase activity.

As another example, thioesterases such as CoA hydrolases catalyze the removal of the CoA moiety. Thioesterases can be promiscuous (Zhuang, et al., Divergence of function in the hot dog fold enzyme superfamily: the bacterial thioesterase YciA, 2008, Biochemistry, 47(9):2789-96). In some embodiments, the promiscuity of thioesterases is exploited by engineering the protein sequence to increase the specificity to the desired substrate. In some embodiments, the (S)-methylmalonyl-CoA hydrolase is a thioesterase comprising an amino acid sequence having at least 65%, at least 70%, at least 75%, at least 80%, at least 85%, at least 90%, at least 95%, at least 96%, at least 97%, at least 98%, at least 99% identity to a sequence selected from SEQ ID 19, 21 or 43 and at least one amino acid difference at a position relative to SEQ ID 19 selected from I39, M45, V60, K71 and V125. In some embodiments, the engineered enzyme has (R)-methylmalonyl-CoA hydrolase activity.

A person of ordinary skill in the art should appreciate that if the crystal structure of an enzyme or of a similar enzyme is known, then the properties of the enzyme may be modified by rational design or by directed evolution (see, for example, Recent advances in rational approaches for enzyme engineering, Comput Struct Biotechnol J. 2012; 2(3) e201209010, US20060160138, WO2003023032, US20080287320 and WO1999029902). For example, WO2013134424 modifies a yeast 3-hydroxyisobutyryl-CoA hydrolase into malonyl-CoA hydrolase to produce malonic acid. Such a modification or improvement in the enzyme properties may arise from the alteration in the structure-function of the enzyme and/or its interaction with other molecules. The interaction of an enzyme with other molecules such as for example the substrate can be quantified by the Michaelis constant (Km), which can be quantified using prior art (see for example, Stryer, Biochemistry, 4^{th} edition, W.H.Freeman, Nelson and Cox, Lenhinger Principles of Biochemistry, 6^{th} edition, W.H. Freeman). The rate of enzymatic activity is defined by kcat, which is the enzyme turnover number. As defined herein, an improvement in the enzyme is to increase the affinity between the enzyme and its substrate, as indicated by lower Km and/or to increase the kcat and/or to increase kcat/Km. Several examples of exploiting the promiscuity of enzymes for synthesizing biochemicals exist in the art. See, for example the description in US20130017593 A1 or WO2009135074 A2 or WO 2010071697 A1. These and other techniques can be used to modify enzymes as suggested herein, for example to enhance the activity of certain enzymes and/or increase the specificity of certain enzymes.

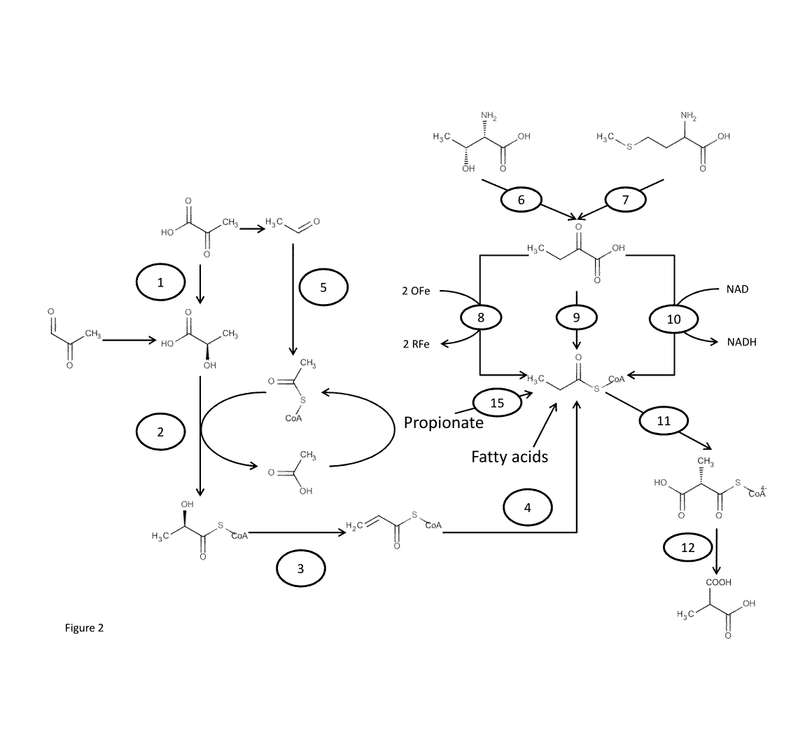

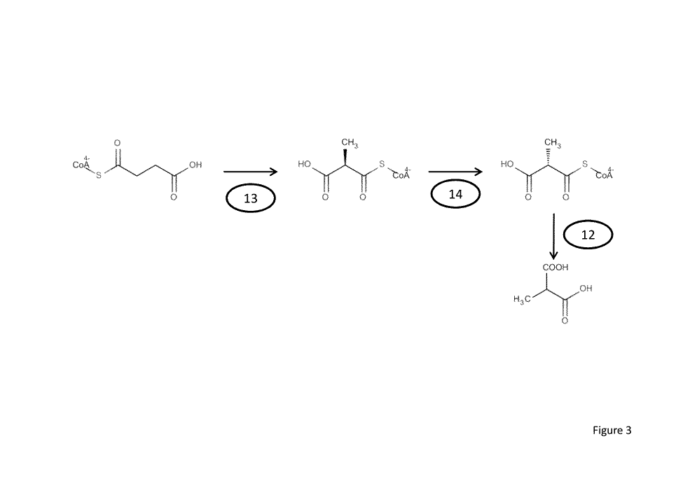

Referring now to FIGS. 2 and 3, the metabolic pathways for producing methylmalonic acid may involve additional processes. For example, as shown in FIG. 2, the metabolic pathway may include one or more of steps 4, 11 and 12. As another example, the metabolic pathway may include one or more of steps 1, 2, 3, 4, 11 and 12. As yet another example, as shown in FIG. 3, the metabolic pathway may include one or more of steps 13, 14 and 12. The metabolic pathways may be implemented in non-natural microorganisms, including yeast and bacteria, which are engineered to produce methylmalonic acid at least using such pathways.

For example, as shown in FIG. 2, in some embodiments, the metabolic pathway includes step 11 in addition to step 12 such that the source of (S)-methylmalonyl-CoA is propionyl-CoA. Propionyl-CoA is carboxylated to (S)-methylmalonyl-CoA by the action of propionyl-CoA carboxylase (Step 11, EC 6.4.1.3). Propionyl-CoA carboxylase is a biotin-dependent, heteromultimeric complex composed of a and _{R} subunits, encoded by pccA and pccB in bacteria such as Myxococcus xanthus (corresponding to the enzyme with the UniProt IDs: Q1DDA2 and Q1DDA0, respectively) or Rhodococcus spheroides (corresponding to the enzyme with the UniProt IDs: Q3J4D9 and Q3J4E3, respectively). In Streptomyces coelicolor, two genes accA1 and accA2 encode for the biotin-binding α-subunit and the pccB encodes for the β-subunit of the propionyl-CoA carboxylase (Rodriguez, E. and H. Gramajo (1999). Genetic and biochemical characterization of the alpha and beta components of a propionyl-CoA carboxylase complex of Streptomyces coelicolor A3(2), Microbiology 145 (Pt 11): 3109-3119). Examples of expressing heterologous propionyl-CoA carboxylase include U.S. Pat. No. 7,413,878 B2; US20020142401 A and WO/2001/031035 A2 for polyketide production. To the inventor's knowledge propionyl-CoA has never been expressed in yeast. In some embodiments, propionyl-CoA carboxylase comprises subunits that have amino acid sequence having at least 65%, at least 70%, at least 75%, at least 80%, at least 85%, at least 90%, at least 95%, at least 96%, at least 97%, at least 98%, at least 99% SEQ ID to 3 or 4, respectively. A method to create a non-natural microorganism harboring propionyl-CoA carboxylase is described in the examples. It will be noted that propionyl-CoA can also be converted into (S)-methylmalonyl-CoA by the action of methylmalonyl-CoA carboxyltransferase (EC 2.1.3.1). Methylmalonyl-CoA carboxyltransferase reversibly converts the transcarboxylation of propionyl-CoA with oxaloacetate to form (S)-methylmalonyl-CoA and pyruvate (Swick and Wood, 1960, The role of transcarboxylation in propionic acid fermentation, Proc Natl Acad Sci USA. 1960 January; 46(1):28-41; Li et al., 2009, Effect of branched-chain amino acids, valine, isoleucine and leucine on the biosythesis of bitespiramycin 4″-O-acylspiramycins, Braz Journal of Microbiol, vol. 40 no. 4 Sao Paulo October/December 2009). It is a large, multi-subunit enzyme that requires the complex assembly of multiple subunits and has not been expressed heterologously.

As another example, in some embodiments, the metabolic pathway includes step 13 in addition to step 12 such that the source of (S)-methylmalonyl-CoA is succinyl-CoA. As is shown in FIG. 3, succinyl-CoA is converted into (R)-methylmalonyl-CoA (Step 13) by methylmalonyl-CoA mutase (EC 5.4.99.2). This enzyme specifically synthesizes (R)-methylmalonyl-CoA, and not (S)-methylmalonyl-CoA, using adenosylcobalamin as a cofactor. This enzyme catalyzes the reversible, stereospecific interconversion of (R)-methylmalonyl-CoA and succinyl-CoA. While in bacteria such as Escherichia coli, the enzyme is encoded by a single gene (scpA), in other archaea such as Metallospora sedula and Propionibacterium freudenreichii, it is encoded by at least two genes to encode for the a (mutA gene) and β (mutB gene) subunits. The UniProt IDs of the corresponding enzyme subunits from Propionibacterium freudenreichii are P11652 (α subunit) and P11653 (β subunit). Table 2 presents exemplary sequences of both kinds of enzymes.

TABLE 2
Methylmalonyl-CoA mutase reaction and UniProt IDs of exemplary proteins that catalyze the reaction

| Enzyme name/ | | |
|---|---|---|
| UniProt ID | EC # | Reaction |
| Methylmalonyl-CoA mutase UniProt ID: Q9GK13 Q23381 P22033 Q8HXX1 P16332 | 5.4.99.2 | 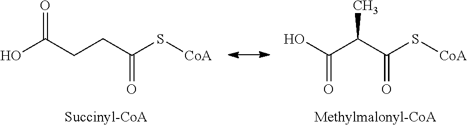 |
| P65486 | | |
| P65485 | | |
| Q8MI68 | | |
| Q5RFN2 | | |
| Q59676 | | |
| P11652 | | |
| Q05064 | | |
| P65488 | | |
| P65487 | | |
| Q59677 | | |
| P11653 | | |
| O86028 | | |
| Q05065 | | |
| P27253 | | |

(R)-methylmalonyl-CoA, thus produced from succinyl-CoA is converted into the S-epimer by methylmalonyl-CoA epimerase (EC 5.1.99.1). The gene encoding for this enzyme is characterized in multi-cellular organisms such as mold and mammals and the protein is localized in the mitochondria of the cells. Some UniProt IDs of exemplary methylmalonyl-CoA epimerases are Q2KIZ3, Q553V2, Q96PE7 and Q9D1I5. Therefore, in some embodiments, this step of the metabolic pathway is facilitated by an enzyme in which the signal sequence that directs the enzyme into the mitochondria is deleted in order to enable the localization of methylmalonyl-CoA epimerase in the cytosol of higher microorganisms. Expression of these genes in Escherichia coli result in an active enzyme, indicating that the enzyme can be constituted in a different host (Dayem et al., Metabolic engineering of a methylmalonyl-CoA mutase-epimerase pathway for complex polyketide biosynthesis in Escherichia coli.” Biochemistry, 2002, 41(16): 5193-5201; Zhang, et al., Investigating the role of native propionyl-CoA and methylmalonyl-CoA metabolism on heterologous polyketide production in Escherichia coli, Biotechnol Bioeng 2010, 105(3): 567-573; US20040185541 A1). In some embodiments, the metabolic pathway is implemented by a non-natural microorganism which harbors at least one gene encoding for methylmalonyl-CoA mutase comprising an amino acid sequence having at least 65%, at least 70%, at least 75%, at least 80%, at least 85%, at least 90%, at least 95%, at least 96%, at least 97%, at least 98%, at least 99% identity to SEQ ID 14. In some embodiments, the non-natural microorganism harbors at least one gene encoding for methylmalonyl-CoA epimerase comprising an amino acid sequence having at least 65%, at least 70%, at least 75%, at least 80%, at least 85%, at least 90%, at least 95%, at least 96%, at least 97%, at least 98%, at least 99% identity to SEQ ID 39. Methods to create such a non-natural microorganism are described in the examples.

In some embodiments, in which propionyl-CoA serves as the source of (S)-methylmalonyl-CoA, the metabolic pathway includes a process in which propionyl-CoA is produced from one or more of propionate, threonine, methionine or pyruvate, as shown in FIG. 2.

Where propionate serves as the source of propionyl-CoA, propionate is converted into propionyl-CoA (Step 15) by propionyl-CoA synthase (EC 6.2.1.17). To the inventor's knowledge, this gene (and enzyme) have never been expressed in yeast before. In Escherichia coli, this enzyme is encoded by the prpE gene. However, the native enzyme is subjected to feedback inhibition by propionylation by propionyl-CoA at lysine 592 (Garrity et al., N-lysine propionylation controls the activity of propionyl-CoA synthetase, J Biol Chem. 2007 Oct. 12; 282(41):30239-45). In some embodiments, therefore the metabolic pathway is implemented in a non-natural microorganism which harbors at least one gene encoding for propionyl-CoA synthase comprising an amino acid sequence having at least 65%, at least 70%, at least 75%, at least 80%, at least 85%, at least 90%, at least 95%, at least 96%, at least 97%, at least 98%, at least 99% identity to SEQ ID 8 or SEQ ID 41. Methods to create a non-natural microorganism harboring propionyl-CoA synthase are illustrated in examples.

Where threonine serves as the source of propionyl-CoA, threonine is dehydrated/deaminated by threonine dehydratase (Step 6, EC 4.3.1.19), which converts threonine into 2-oxobutanoate. In Escherichia coli, this enzyme is encoded by the catabolic tdcB (b3117) or biosynthetic ilvA (b3772) genes. Threonine is produced from aspartate and the first step in this pathway, aspartate kinase, is subject to feedback inhibition by threonine. The mechanism for feedback inhibition is well-studied and in yeast (Arevalo-Rodriguez et al., Mutations that cause threonine sensitivity identify catalytic and regulatory regions of the aspartate kinase of Saccharomyces cerevisiae, Yeast, 1999, 1(13): 1331-1345) and bacteria (Yoshida A, Tomita T, Kuzuyama T, Nishiyama M: Mechanism of concerted inhibition of alpha2beta2-type hetero-oligomeric aspartate kinase from Corynebacterium glutamicum. The Journal of biological chemistry 2010, 285(35):27477-27486). In some embodiments, the metabolic pathway is implemented by a non-natural microorganism created by enhancing the activity of EC 4.3.1.19 by introducing an enzyme comprising an amino acid sequence having at least 65%, at least 70%, at least 75%, at least 80%, at least 85%, at least 90%, at least 95%, at least 96%, at least 97%, at least 98%, at least 99% identity to a sequence selected from SEQ ID 44 SEQ ID 45 or SEQ ID 46.

Where methionine serves as the source of propionyl-CoA, the metabolic pathway may involve synthesizing 2-oxobutanoate from methionine by the action of methionine-γ-lyase (Step 7, EC 4.4.1.11). While there are some reports of this enzyme from archaea and eukaryota, this enzyme is more common in bacteria. For example, mdeA gene from Pseudomonas putida encodes for this enzyme and catalyzes the α,γ-elimination and γ-replacement reactions of L-methionine and its S-substituted derivatives. In some embodiments, the metabolic pathway is implemented in a microorganism which is engineered with genes that encode for threonine hydratase/deaminase or methionine-γ-lyase to render the conversion of threonine or methionine into 2-oxobutanoate. In some embodiments, the native aspartate kinase in the microorganism is replaced with feedback-resistant aspartate kinase to decouple threonine/methionine production from regulation.

2-oxobutanoate produced from step 6 or step 7 is oxidatively decarboxylated to propanoyl-CoA. This reaction is catalyzed by 2-oxobutanoate formate-lyase (Step 9, EC 2.3.1.-). In Escherichia coli, this enzyme is encoded by the tdcE (b3114) gene, which encodes for the inactive enzyme that is activated by pflA (b0902) gene product. The functioning of this enzyme is similar to that of pyruvate formate-lyase. Since pyruvate formate lyase is sensitive to oxygen, the grcA (b2579) gene from Escherichia coli replaces an oxidatively damaged pyruvate formate-lyase subunit. The auxiliary genes needed to sustain the activity of 2-oxobutanoate formate-lyase, pflA and grcA, are co-expressed with tdcE and the ensuing formate is oxidized to carbon dioxide by formate dehydrogenase such as for example, EC 1.2.1.2. In some embodiments, the metabolic pathway is implemented in a non-natural microorganism which is created by enhancing the activity of 2-oxobutanoate formate-lyase by introducing an enzyme comprising an amino acid sequence having at least 65%, at least 70%, at least 75%, at least 80%, at least 85%, at least 90%, at least 95%, at least 96%, at least 97%, at least 98%, at least 99% identity to a sequence selected from SEQ ID 47, 48 or 49.

2-oxobutanoate is decarboxylated by the ydbK (b1378) gene product in E. coli, 2-oxobutanoate synthase (Step 8, EC 1.2.7.1). Based on sequence similarity, YdbK is predicted to function as 2-oxoacid:flavodoxin oxidoreductase synthase (Nakayama et al., 2013, Escherichia coli pyruvate:flavodoxin oxidoreductase, YdbK—regulation of expression and biological roles in protection against oxidative stress. Genes Genet Syst. 2013; 88(3):175-88). Oxidative decarboxylation of 2-oxobutanoate is also catalyzed by branched-chain 2-oxo acid dehydrogenases (Step 10, EC 1.2.4.4). In some embodiments, the metabolic pathway is implemented in a non-natural microorganism which is created by enhancing the activity of 2-oxobutanoate synthase by introducing an enzyme comprising an amino acid sequence having at least 65%, at least 70%, at least 75%, at least 80%, at least 85%, at least 90%, at least 95%, at least 96%, at least 97%, at least 98%, at least 99% identity to a sequence selected from SEQ ID 56 or SEQ ID 57.

In bacteria, the pyruvate dehydrogenase enzyme complex is also able to recognize 2-oxobutanoate as the substrate, and decarboxylate it to propanoyl-CoA. In some embodiments, the metabolic pathway is implemented in a microorganism, which is engineered with the genes that encode for at least one of 2-oxobutanoate synthase, 2-oxobutanoate formate-lyase and 2-oxo acid dehydrogenase enzymes.

In some embodiments, propanoyl-CoA is produced from pyruvate according to the sequence of reactions shown in FIG.2. Pyruvate is reduced to (R)-lactate by the action of D-lactate dehydrogenase (Step 1, EC 1.1.1.28) commonly using NADH as the reducing agent. An example of a gene that encodes for D-lactate dehydrogenase is ldhD from Lactobacillus plantarum (UniProt ID of the corresponding enzyme: Q88VJ2) or the ldhA (b1380) from Escherichia coli (UniProt ID of the corresponding enzyme: P52643). (R)-lactate is also produced by the hydrolysis of methylglyoxal for example by glyoxylase III (EC 4.2.1.130) or by glyoxylase I (EC 4.4.1.5). In some embodiments, the metabolic pathway is implemented by the non-natural microorganism which is created by enhancing the activity of D-lactate dehydrogenase by introducing an enzyme comprising an amino acid sequence having at least 65%, at least 70%, at least 75%, at least 80%, at least 85%, at least 90%, at least 95%, at least 96%, at least 97%, at least 98%, at least 99% identity to a sequence selected from SEQ ID 61 or SEQ ID 62. Pyruvate is reduced to (S)-lactate by the action of (S)-lactate dehydrogenase (EC 1.1.1.27) commonly using NADH as the reducing agent. An example of a gene that encodes for (S)-lactate dehydrogenase is ldh gene of Lactobacillus casei (UniProt ID of the corresponding enzyme: P00343). In some embodiments, the metabolic pathway is implemented in the non-natural microorganism which is created by enhancing the activity of (S)-lactate dehydrogenase by introducing an enzyme comprising an amino acid sequence having at least 65%, at least 70%, at least 75%, at least 80%, at least 85%, at least 90%, at least 95%, at least 96%, at least 97%, at least 98%, at least 99% identity to a sequence selected from SEQ ID 63. Methods to engineer (R)-lactate dehydrogenase activity or (S)-lactate dehydrogenase activity in an organism are described in the examples.

(R)-lactate or (S)-lactate formed in Step 1 is converted into (R)-lactoyl-CoA or (S)-lactoyl-CoA, respectively by the action of lactate CoA transferase (Step 2, EC 2.8.3.-). Lactate CoA-transferase is one of the key enzymes of the propionate fermentation pathway in anaerobic microorganisms such as Clostridium propionicum and Megasphaera elsdenii. When using lactate as a substrate the enzyme catalyzes an early step in the pathway yielding lactyl-CoA. The pct gene from Clostridium propionicum encodes for lactoyl-CoA transferase. This enzyme can use propanoyl-CoA as well as acetyl-CoA as the donor of Coenzyme A. The pct gene from Megasphaera elsdenii was shown to have a lower Km for (R)-lactate than for (S)-lactate and was used to produce 1,2-propanediol by engineering E. coli (Niu and Guo, 2015, Stereospecific Microbial Conversion of Lactic Acid into 1,2-Propanediol, ACS Synthetic Biology, 4(4): 378-382). In some embodiments, the metabolic pathway is implemented by the non-natural microorganism which is created by enhancing the activity of lactate-CoA transferase by introducing an enzyme comprising an amino acid sequence having at least 65%, at least 70%, at least 75%, at least 80%, at least 85%, at least 90%, at least 95%, at least 96%, at least 97%, at least 98%, at least 99% identity to a sequence selected from SEQ ID 64 or SEQ ID 65 or SEQ ID 66.

The CoA donating entity is acetyl-CoA, which is converted to acetate. Acetate is recycled back to acetyl-CoA by the action of acetyl-CoA synthetase (EC 6.2.1.1). In some embodiments, the metabolic pathway is implemented in a non-natural microorganism which is created by enhancing the activity of acetyl-CoA synthetase by introducing an enzyme comprising an amino acid sequence having at least 65%, at least 70%, at least 75%, at least 80%, at least 85%, at least 90%, at least 95%, at least 96%, at least 97%, at least 98%, at least 99% identity to a sequence selected from SEQ ID 67 or SEQ ID 68. Acetyl-CoA is also produced from acetaldehyde by the action of acetaldehyde dehydrogenase (Step 5, EC 1.2.1.10). This enzyme can catalyze the reversible reaction shown by step 5. An example of a gene that encodes for acetaldehyde dehydrogenase is adhE (b1241) in Escherichia coli (UniProt ID of the corresponding enzyme: P0A9Q7). The CoA donating entity is propionyl-CoA, which is converted to propionate. Propionate is recycled back to propionyl-CoA by the action of propionyl-CoA synthase. In some embodiments, the metabolic pathway is implemented in a non-natural microorganism which is created by enhancing the activity of propionyl-CoA synthetase by introducing an enzyme comprising an amino acid sequence having at least 65%, at least 70%, at least 75%, at least 80%, at least 85%, at least 90%, at least 95%, at least 96%, at least 97%, at least 98%, at least 99% identity to a sequence selected from SEQ ID 8 or SEQ ID 41 or SEQ ID 42.

Lactoyl-CoA is dehydrated to acryloyl-CoA by the action of lactoyl-CoA dehydratase (Step 3, EC 4.2.1.54). Lactyl-CoA dehydratase is one of the enzymes in the propionate fermentation pathway. The enzyme complex is composed of two proteins, EI (encoded by lcdC) is the activator protein and EII (lcdAB) is the actual dehydratase (Schweiger and Buckel, 1984, On the dehydration of (R)-lactate in the fermentation of alanine to propionate by Clostridium propionicum, FEBS 171(1): 79-84; Hofmeister and Buckel, 1992, (R)-Lactyl-CoA dehydratase from Clostridium propionicum, Eur J Biochem, 206:547-552). The three genes provide for activity and the genes from Clostridium propionicum were shown to function heterologously in Escherichia coli and participate in fermenting lactate to propanoate (Kandasamy et al., 2013, Engineering Escherichia coli with acrylate pathway genes for propionic acid synthesis and its impact on mixed-acid fermentation, Appl Microbiol Biotechnol., 97(3):1191-1200). Similarly, EP1343874 B1 and related patents teaches the expression of the genes that encode for lactoyl-CoA dehydratase from M. elsdenii in yeast. The engineered yeast was used to produce 3-hydroxypropionic acid. In some embodiments, the non-natural microorganism is created by enhancing the activity of lactate-CoA dehydratase by introducing enzymes comprising an amino acid sequence having at least 65%, at least 70%, at least 75%, at least 80%, at least 85%, at least 90%, at least 95%, at least 96%, at least 97%, at least 98%, at least 99% identity to a sequences selected from SEQ ID 69, SEQ ID 70 and SEQ ID 71.

Acryloyl-CoA is reduced to propanoyl-CoA by the action of acryloyl-CoA reductase (Step 4, EC 1.3.1.95). This heterohexadecameric enzyme from C. propionicum catalyzes the irreversible, NADH-dependent conversion of acrylyl-CoA (acryloyl-CoA) to propionyl-CoA. It is a complex of acryloyl-CoA reductase (encoded by acrC) and an electron-transfer flavoprotein (encoded by acrA and acrB). These genes, from Clostridium propionicum were shown to function heterologously in Escherichia coli and participate in fermenting lactate to propanoate (Kandasamy et al., 2013). Another class of acryloyl-CoA reductase is from Rhodobacter sphaeroides and Ruegeria pomeroyi which uses NADPH as the reducing agent (Asao and Alber, 2013, Acrylyl-coenzyme A reductase, an enzyme involved in the assimilation of 3-hydroxypropionate by Rhodobacter sphaeroides, J. Bacteriology, 195(20):4716-4725). In some embodiments, the non-natural microorganism is created by enhancing the activity of acryloyl-CoA reductase by introducing enzyme comprising an amino acid sequence having at least 65%, at least 70%, at least 75%, at least 80%, at least 85%, at least 90%, at least 95%, at least 96%, at least 97%, at least 98%, at least 99% identity to a sequences selected from SEQ ID 72 and SEQ ID 73.

In some embodiments, the metabolic pathway is implemented (and methylmalonic acid is produced) in a microorganism engineered with genes encoding for at least one or more of the enzymes described above, including one or more of D-lactate dehydrogenase, L-lactate dehydrogenase, lactate CoA transferase, lactoyl-CoA dehydratase, acryloyl-CoA reductase, acetyl-CoA synthase, propionyl-CoA synthase, propionyl-CoA carboxlase and methylmalonyl-CoA hydrolase.

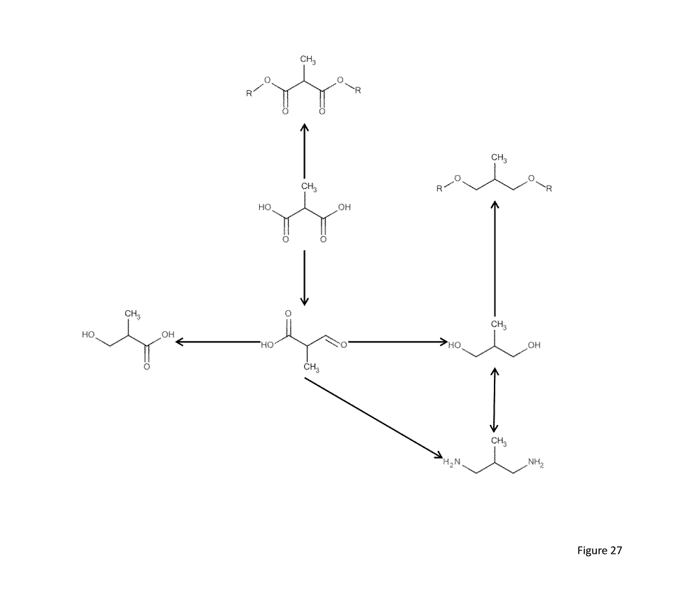

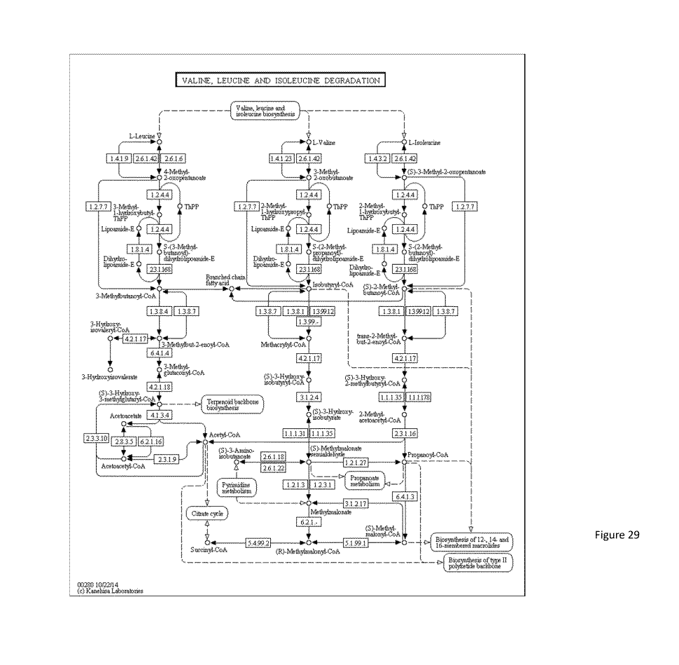

In some embodiments, the metabolic pathway involves production of methylmalonic acid from L-glutamate, according to FIG. 27. The committed reaction step in this sequence is catalyzed by glutamate mutase (Step 15, EC 5.4.99.1), which converts glutamate to 3-methylaspartate. Glutamate mutase is a adenosylcobalamin-dependent enzyme that rearranges glutamate into methylaspartate. The enzyme from Clostridium cochlearium is a heterotetramer that are bound by Vitamin B 12 (Zelder, et al., 1994, Characterization of the coenzyme-B12-dependent glutamate mutase from Clostridium cochlearium produced in Escherichia coli, Eur J Biochem 226(2): 577-585). Exemplary enzymes that can catalyze this reaction have UniProt IDs P80077 and P80078. In some embodiments, the metabolic pathway is implemented in a non-natural microorganism created by enhancing the activity of glutamate synthase by introducing enzyme comprising an amino acid sequence having at least 65%, at least 70%, at least 75%, at least 80%, at least 85%, at least 90%, at least 95%, at least 96%, at least 97%, at least 98%, at least 99% identity to a sequences selected from sequences associated with UniProt IDs P80077 and P80078. 3-methylaspartate transaminase (Step 16, EC 2.6.1.-) deaminates 3-methylaspartate to methyloxaloacetate with the concomitant conversion of a-ketoglutarate to glutamate. The transamination is also driven by other 2-oxo acid/amino acid pairs such as pyruvate/alanine, oxaloacetate/aspartate, etc. Methyloxaloacetate formed is decarboxylated by 2-oxo acid decarboxylase (EC 4.1.1.-) to form methylmalonic semialdehyde. Examples of such 2-oxo acid decarboxylases are prevalent in the Ehrlich pathways. In these pathways, amino acids are transaminated to the corresponding 2-oxo acids, which are then decarboxylated by the decarboxylases into aldehydes. The aldehydes are converted into alcohols, known as fusel alcohols. Examples of genes encoding such decarboxylases are PDC1, PDC5, PDC6, ARO10, and THI3 from Saccharomyces cerevisiae. In the referenced pathway (See FIG. 29), methylmalonic semialdehyde is converted into 3-hydroxy-2-methylpropanoic acid by the action of methylmalonic semialdehyde dehydrogenase (EC 1.2.1.27). Exemplary enzymes that catalyze this reaction are identified by their UniProt IDs: P28810 and Q8VUC5. Methylmalonic semialdehyde is oxidized to methylmalonic acid by the action of aldehyde dehydrogenases (EC 1.2.1.-) using NAD(P) as the cofactor.

In some embodiments, methylmalonic acid is reduced to methylmalonic semialdehyde by the action of methylmalonic semialdehyde dehydrogenase. The reducing agent in this conversion is NADH or NADPH. Methylmalonic semialdehyde is also converted to 2-methylpropane-1,3-diol by the action of methylmalonic semialdehyde dehydrogenase and alcohol dehydrogenase. In some embodiments, methylmalonic semialdehyde is converted to 2-methylpropane-1,3-diamine by the action of transaminases (EC 2.6.1.-). In some embodiments of the invention, 2-methylpropane-1,3-diol is converted to the corresponding ester by the action of alcohol acyl transferases (EC. 2.3.1.-).

### Choice of Host Microorganisms

Embodiments according to the specification encompass microorganisms such as yeast and bacteria that are engineered to include one or more of the aforementioned enzymes and produce methylmalonic acid via a metabolic pathway for example according to one or more of the pathways provided herein. In some embodiments, one or more of the aforementioned enzymes is engineered to have a Km that is less than the Km of the corresponding wild type enzyme. In some embodiments, one or more of the aforementioned enzymes is engineered to have a Km that is less than about half of the Km of the corresponding wild type enzyme. In some embodiments, the microorganism is engineered by introducing heterologous genes either from a plasmid or by integrating in the chromosome. In some embodiments, the microorganism is a bacteria chosen from one or more of: Acetobacterium, Aerobacter, Agrobacterium, Alcaligenes, Azotobacter, Bacillus, Clostridium, Corynebacterium, Escherichia, Flavobacterium, Lactobacillus, Micromonospora, Mycobacterium, Nocardia, Propionibacterium, Protaminobacter, Proteus, Pseudomonas, Rhizobium, Salmonella, Serratia, Streptomyces, Streptococcus and Xanthomonas. In some embodiments, the microorganism is a yeast chosen from one or more of: Candida, Pichia, Kluyveromyces, Saccharomyces, Debaromyces, Hansenula, Pachysolen and Yarrowia. In some embodiments, the microorganism is a methanogenic archaea such as Methanococcus maripaludis. In some embodiments, the microorganism is a filamentous fungus chosen from one or more of: Aspergillus, Penicillium, Acremonium, Fusarium, Neospora and Mucor.

### Metabolic Engineering of Bacteria

In addition, or in the alternative (if the microorganism produces methylmalonic acid), to including one or more of the metabolic pathway enzymes described above in a bacteria, the yield (efficiency of conversion) of methylmalonate from substrates may be increased by eliminating pathways that compete for the substrate to produce by-products. In some embodiments, the genes that encode for enzymes that catalyze the conversion of pyruvate into by-products such as lactate, acetate and formate is minimized in the bacterial microorganism. For example, in Escherichia coli, the conversion of pyruvate to lactate is catalyzed by lactate dehydrogenase and is encoded by lldD gene (b3605) and ldhA gene (b1380). The conversion of pyruvate to formate is catalyzed by pyruvate formate-lyase. This enzyme is encoded by pflB gene (b0903) and is activated by pflA gene (b0902) in Escherichia coli. The conversion of pyruvate to acetate occurs by the two routes. The first pathway utilizes a single step conversion catalyzed by pyruvate oxidase (EC 1.2.5.1), encoded by the poxB gene (b0871) in Escherichia coli. The second pathway uses acetyl-CoA as an intermediate. Acetyl-CoA is converted to acetylphosphate by phosphotransacetylase (EC 2.3.1.8), which is encoded by the pta gene in (b2297) Escherichia coli. Acetylphosphate is converted to acetate by liberating phosphate by acetate kinase (EC 2.7.2.1) and is encoded by ackA gene (b2296) in Escherichia coli. In order to enhance the availability of succinyl-CoA for methylmalonate production, the conversion of succinate to succinyl-CoA is enhanced by overexpressing succinyl-CoA synthase. In Escherichia coli this enzyme is encoded by the b0728 and b0729 genes.

Further, the transport of methylmalonic acid in bacteria is mediated by a dicarboxylic acid transporter. Examples of several dicarboxylic acid transporters are reported in literature. The genes in Escherichia coli that catalyze the transport are encoded by the genes listed in Table 3.

TABLE 3
Exemplary dicarboxylic acid transporters in Escherichia coli

| Gene Name | Enzyme |
|---|---|
| b1206 | C4 dicarboxylate/C4 monocarboxylate transporter |
| b3528 | C4 dicarboxylate/orotate: H+ symporter |
| b4138 | dicarboxylate transporter |
| b4123 | dicarboxylate transporter |
| b0621 | dicarboxylate transporter |

In some embodiments, the bacterial microorganism is in addition or in the alternative engineered by down-regulating at least one of the genes that encode for the enzymes that catalyze the conversion of pyruvate into acetate, lactate or formate. In some embodiments, the bacterial microorganism is engineered by the introduction of anaplerotic enzymes such as pyruvate carboxylase and ATP-generating phosphoenolpyruvate carboxykinase (Uniprot ID: A6VKV4). In some embodiments, the non-natural microorganism is created by enhancing the activity of ATP-generating phosphoenolpyruvate carboxykinase by introducing an enzyme comprising an amino acid sequence having at least 65%, at least 70%, at least 75%, at least 80%, at least 85%, at least 90%, at least 95%, at least 96%, at least 97%, at least 98%, at least 99% identity to a sequence selected from SEQ ID 50.

In some embodiments, wherein the microorganism utilizes the phosphoenolpyruvate-dependent phosphotransferase system for the uptake of hexose, the bacterial microorganism is engineer or further engineered to have an inactivated phosphotransferase system and enhanced hexokinase activity. In some embodiments, the bacterial microorganism is engineered with enhanced dicarboxylic acid transporter activity.

### Metabolic Engineering of Yeast

Eukaryotic metabolism is compartmentalized and therefore, the regulatory mechanisms significantly differ from those in bacteria. In addition, or in the alternative (if the microorganism produces methylmalonic acid), to including one or more of the metabolic pathway enzymes described above in yeast, in order to increase the yield of methylmalonate, the conversion of pyruvate to ethanol is minimized by deleting at least one of pyruvate decarboxylase or alcohol dehydrogenase reactions. Since there are multiple genes that encode for each of these reactions, the activity of these enzymes is minimized by down-regulating the gene expression either by deletion of or by decreasing the promoter strength of the genes. In eukaryotes, pyruvate is transported from cytosol into mitochondria. The transport is mediated by pyruvate transporter. The activity of the pyruvate transporter can be attenuated by decreasing the expression of the gene that encodes for it. For example in S. cerevisiae, a gene that encodes for the pyruvate transport into the mitochondria could be YIL006W.

Anaplerotic reactions in eukaryotes are predominantly in the mitochondria. Expressing ATP-generating phosphoenolpyruvate carboxykinase (EC 4.1.1.49) in the cytosol will provide oxaloacetate for threonine/methionine synthesis along with the generation of ATP. An example of a gene encoding for this enzyme is pckA from Actinobacillus succinogenes (UniProt ID: A6VKV4). In some embodiments, the non-natural microorganism is created by enhancing the activity of ATP-generating phosphoenolpyruvate carboxykinase by introducing an enzyme comprising an amino acid sequence having at least 65%, at least 70%, at least 75%, at least 80%, at least 85%, at least 90%, at least 95%, at least 96%, at least 97%, at least 98%, at least 99% identity to a sequence selected from SEQ ID 50. Methods to create a microorganism with enhanced ATP-generating phosphoenolpyruvate carboxykinase activity are described in the examples.

In some embodiments, the non-natural microorganism is created by enhancing the activity of pyridine nucleotide transhydrogenase (EC 1.6.1.2 or EC 1.6.1.3) by introducing an enzyme comprising an amino acid sequence having at least 65%, at least 70%, at least 75%, at least 80%, at least 85%, at least 90%, at least 95%, at least 96%, at least 97%, at least 98%, at least 99% identity to a sequence selected from SEQ ID 52 or SEQ ID 53. Methods to create a microorganism with enhanced transhydrogenase activity are described in the examples.

The excretion of methylmalonate out of the cell is mediated by dicarboxylic acid transporters. The first dicarboxylic acid transporter in yeast was reported in Kluyveromyces lactis, which transported malate, succinate, fumarate and α-ketoglutarate. Several transporters have been described since then (Casal M, Paiva S, Queiros O, Soares-Silva I: Transport of carboxylic acids in yeasts. FEMS microbiology reviews 2008, 32(6):974-994; Grobler J, Bauer F, Subden R E, Van Vuuren H J: The mael gene of Schizosaccharomyces pombe encodes a permease for malate and other C4 dicarboxylic acids. Yeast 1995, 11(15):1485-1491, both of which are herein incorporated by reference in their entirety). For example, the malic acid permease (MAE1) from Schizosaccharomyces pombe encodes for a permease for dicarboxylic acids, including malonic acid. Physiological characterization of S. cerevisiae strain transformed with S. pombe MAE1 gene (GenBank ID: NM_001020205) enabled the transport of monoanionic form of acids.

Detailed information on the transporters identified is reviewed by Casal et al (supra) and also thoroughly documented in Saccharomyces cerevisiae at http://genolevures.org/yeti.html. Exemplary dicarboxylic acid transporters are shown in Table 4.

TABLE 4
Exemplary dicarboxylic acid transporters in yeasts

| gene name | putative substrate | Organism |
|---|---|---|
| ODC2, ODC2 | 2-OxoDiCarboxylate | Saccharomyces cerevisiae |
| ODC1, ODC1 | 2-OxoDiCarboxylate | Saccharomyces cerevisiae |
| DIC1/DTP, DIC1 | Dicarboxylate | Saccharomyces cerevisiae |
| DIP5, DIP5 | Dicarboxylic Amino | Saccharomyces cerevisiae |
| | Acids | |
| MAE1 | Malic acid | Schizosaccharomyces pombe |
| ME | Malic acid | Candida utilis |
| KMS3 | malic acid | Kluyveromyces marxianus |

In some embodiments, a eukaryotic microorganism is engineered by down-regulating the transport of pyruvate into the mitochondria by attenuating the expression of the transporter gene. In some embodiments, the conversion of pyruvate to ethanol is minimized by down-regulating the activity of pyruvate decarboxylase/alcohol dehydrogenase enzymes. In some embodiments, the energy efficiency of the production of aspartate is enhanced by introducing ATP-generating phosphoenolpyruvate carboxykinase. In some embodiments, the eukaryotic microorganism is engineered by enhancing the dicarboxylic acid export activity.

The following examples are provided only as a means to further illustrate the invention and not to restrict it in any manner.

### EXAMPLES

### Experimental Methods

### Detection of Methylmalonic Acid

LC-MS analysis was conducted on an ultrahigh pressure LC system (Shimadzu UFLC XR) online with a triple stage quadrupole mass spectrometer (5500 QTRAP, AB Sciex, Washington, DC, USA) equipped with a 100×2.1 mm inner diameter, 5 μm, HYPERCARB column. The column temperature was maintained at 35° C. An injection volume of 10 μL was chosen. A linear binary gradient at a flow rate of 0.3 mL/min with water and acetonitrile as solvents were used, with each containing 0.1% formic acid. The initial gradient concentration was 2% acetonitrile, which was kept constant for 1 min, linearly increased to 98% in 3.50 min, kept constant for 1 min, and followed by column equilibration steps. The LC column eluate entered the electrospray ionization (ESI) interface of the mass spectrometer operating in the negative ion mode. The MS parameters were sheath gas (N₂ 99.99%, flow rate=25 units) with vaporization temperature of 350° C. and collision cell exit potential of −9 V, spray voltage of 4.5 kV, entrance potential of −10 V and declustering potential of −30 V. Acquisition was carried out in the MRM mode to achieve maximal sensitivity and reliable quantitation over several orders of magnitude of compound abundance. Q1, precursor molecule, of 116.9 with a Q3 transition of 73 (CE-15) and 55 (CE-30) were selected and conditions optimized using Analyst software. Concentrations of were calculated based on peak areas integrated by MultiQuant (version 2.0.2) compared to a standard curve of known concentration using authentic methylmalonic acid. Liquid chromatography retention time was used to distinguish methylmalonic acid from succinate by using standards under the above conditions.

### Example 1

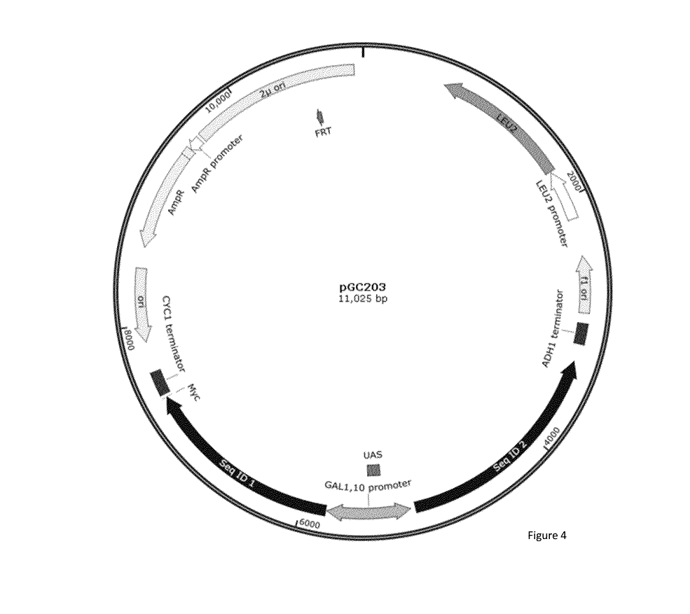

This example describes yeast cells that are engineered to produce methylmalonic acid. DNA was synthesized de novo (GenScript, Piscataway, N.J.) according to sequence ID 1 and Sequence ID 2 and was cloned into yeast/E. coli shuttle vector with ampicillin resistance, leucine marker and bidirectional Gal1/Gal10 promoters for expressing the genes in yeast. The de novo synthesized DNA according to sequence ID 1 contained restriction sites for BamHI and XhoI and the de novo synthesized DNA according to sequence ID 2 contained restriction sites for SpeI and SacI. The shuttle vector also contained these restriction sites after Gal1 and Gal10 promoter regions, respectively. The de novo synthesized DNA and the plasmid were digested with the corresponding restriction enzymes to the construct the plasmid pGC203 shown in FIG. 4.

DNA encoding for propanoyl-CoA synthase was amplified from the genomic DNA of E. coli using the primers with the sequence ID 5 and sequence ID 6. The resulting DNA fragment was restriction digested with EcoRI and SacI enzymes and ligated into a yeast/E. coli shuttle vector with ampicillin resistance, uracil marker and Gal10 promoter for expressing the gene in yeast. The DNA encoding for propanoyl-CoA synthase corresponds to Sequence ID 7.

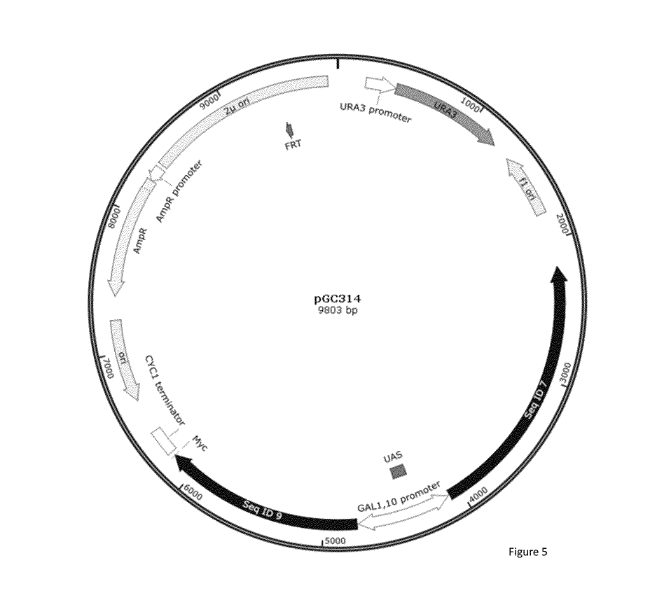

The mitochondrial signal sequence was identified by TargetP1.1 (Emanuelsson O, Nielsen H, Brunak S, von Heijne G: Predicting subcellular localization of proteins based on their N-terminal amino acid sequence. Journal of molecular biology 2000, 300(4):1005-1016) to be the first 31 amino acids, which was replaced with ATG. DNA corresponding to Sequence ID 9 and was amplified from the genome of Saccharomyces cerevisiae using the primers with Sequence ID 11 and Sequence ID 12. This amplified DNA fragment was digested with BamHI and XhoI and ligated into the yeast/E. coli shuttle vector with ampicillin resistance, uracil marker and Gal1 promoter, which was also digested with the same enzymes. The resulting plasmid contains the two genes is shown in FIG. 5.

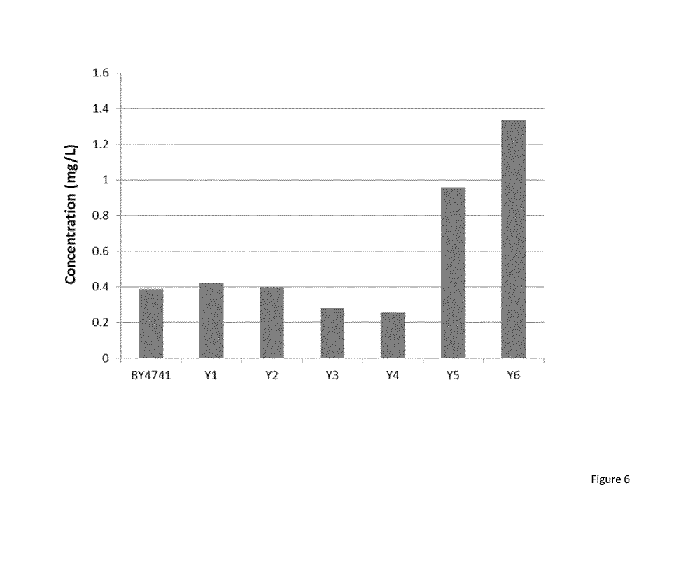

The two plasmids, pGC203 and pGC314 were transformed into S. cerevisiae strain BY4741 using standard protocols (R. D. Gietz and R. A. Woods, Methods Enzymol., 2002, 350, 87). The transformed yeast strain (Y6) containing the two plasmids was grown in synthetic defined medium lacking leucine and uracil. As a control, BY4741 transformed with the two shuttle vectors without the genes of interest (Y1) was also grown in synthetic defined medium lacking leucine and uracil. In this manner, only the gene corresponding to Sequence ID 1 (Y2), genes corresponding to Sequence ID 1 and Sequence ID 2 (Y3), genes corresponding to Sequence ID 1 and Sequence ID 9 (Y4) and genes corresponding to Sequence ID 1, Sequence ID 7 and Sequence ID 9 (Y5) were introduced into yeast. The six yeasts were grown in 250 mL shake flasks at 30° C. in 25 mL of synthetic defined lacking uracil and leucine supplemented with 10 g/L of galactose as carbon source and inducer. The flasks were shaken at 200 rpm for 55.5 h. The wild-type and Y1 control produced small quantities of methylmalonic acid, which is attributed to the promiscuous, residual activity of 3-hydroxyisobutyryl-CoA. As indicated in FIG. 6, by introducing the genes for Steps 11 and 12, methylmalonic acid production by recombinant yeast increased.

### Example 2

This example demonstrates the production of methylmalonic acid by engineered bacteria.

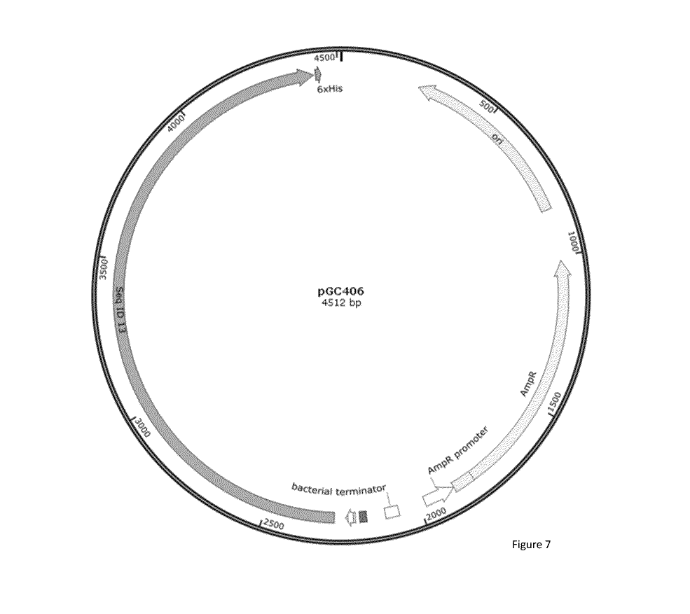

DNA sequence corresponding to Sequence ID 13 was amplified from the genomic DNA of Escherichia coli using primers corresponding to Sequence ID15 and Sequence ID 16. The amplified DNA was digested with BglII and XhoI restriction enzymes and was ligated into pUC-based plasmid which was also digested with the same enzymes. This plasmid, designated pGC406 and shown in FIG. 7, has the cloned DNA was expressed under the control of the lac promoter.

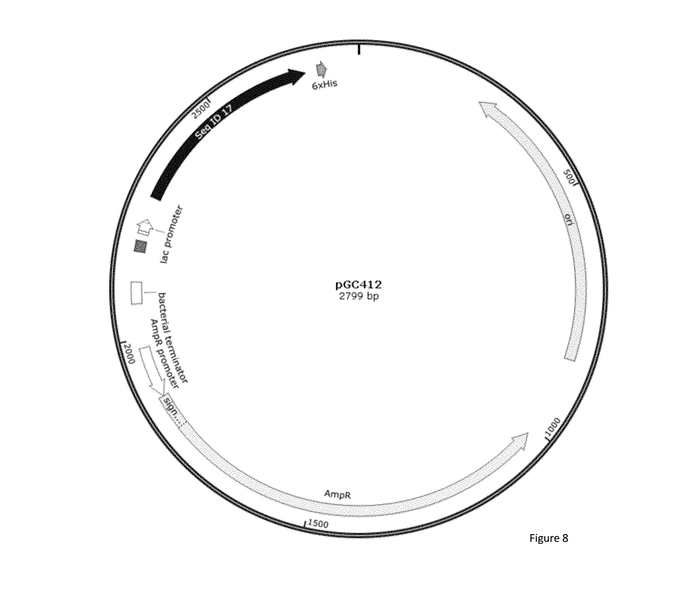

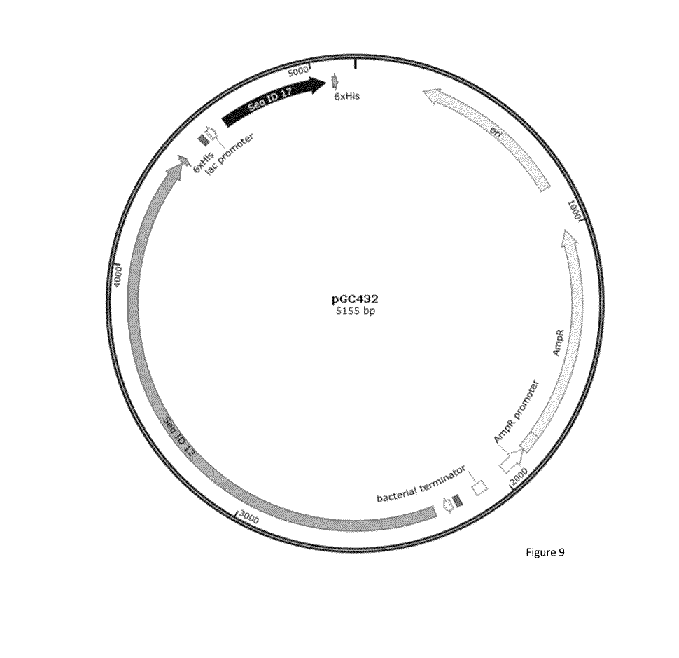

DNA sequence corresponding to Sequence ID 17 was synthesized as gBlocks (Integrated DNA Technologies, Coralville, Iowa) and was restriction digested with BamHI and XhoI restriction enzymes. The DNA fragment was ligated into pUC-based plasmid which was also digested with the same restriction enzymes such that the DNA is expressed under the control of lac promoter. The plasmid is designated pGC412 and is shown in FIG. 8. pGC412 was digested with EcoRI and XbaI restriction enzymes, whose cut sites were located upstream of the transcription unit of the DNA corresponding to Sequence ID 17. A 2.3 kb DNA fragment that contained the corresponding to Sequence ID 13 was liberated from pGC406 using restriction enzymes EcoRI and SpeI. The two DNA fragments were ligated by taking advantage of the compatible sticky ends of XbaI and SpeI and upon ligation, results in neither XbaI nor SpeI sites. The resulting plasmid that contained Sequence ID 17 and Sequence ID 13 is designated pGC432 and is shown in FIG. 9.

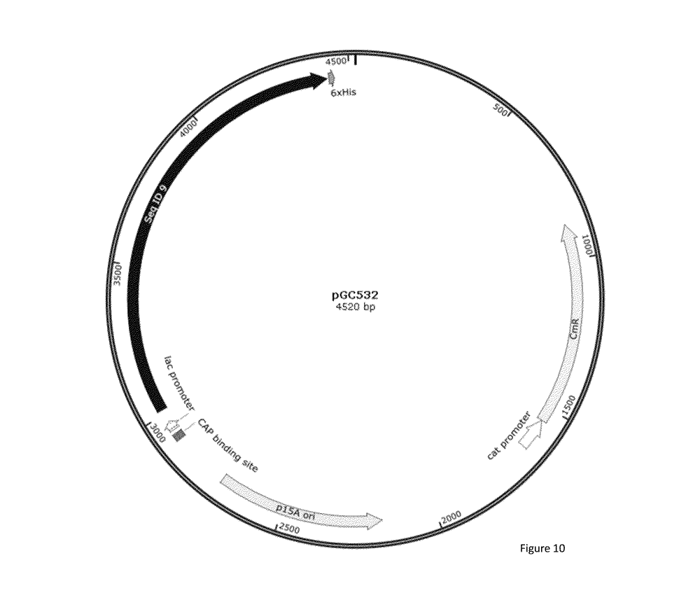

DNA corresponding to Sequence ID 9 and was amplified from the genome of Saccharomyces cerevisiae using the primers with Sequence ID 11 and Sequence ID 12. This DNA was restriction digested with BamHI and XhoI and ligated into pACYC-based plasmid which was also digested with the same restriction enzymes. The plasmid was designated pGC532 and is shown in FIG. 10.

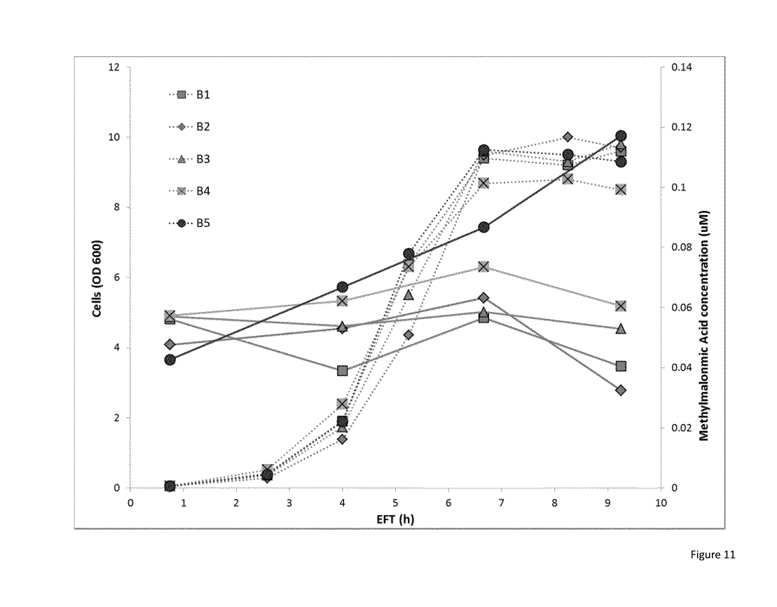

The two plasmids, pGC432 and pGC532, were transformed into Escherichia coli BW25113 using electroporation and the resulting strain is called B5. In this manner, only the gene corresponding to Sequence ID 9 (B4), genes corresponding to Sequence ID 13 and Sequence ID 17 (B3) and the gene corresponding to sequence ID 13 (B2) were introduced into the bacterium and compared against the control which contained empty plasmids (B 1) for methylmalonic acid production. The bacteria were grown in a medium that contained M9 minimal medium (50%) and LB broth (50%) at a starting pH of 7. The plasmids were maintained by adding 100 mg/L of Ampicillin and 50 mg/L of chloramphenicol. 18 g/L of glucose was used as the carbon source. The bacterial cultures were grown in 250 mL shakeflasks with 25 mL working volume at 37° C. by shaking at 200 rpm. The concentration of methylmalonic acid was detected in all the strains at the beginning of the experiment. While there was no significant change in the methylmalonic acid concentration in the strains B1-B4, E. coli containing Steps 13, 14 and 12 produced methylmalonic acid (FIG. 11).

### Example 3

This example demonstrates the functional expression of methylmalonyl-CoA hydrolases in bacteria.

DNA sequence corresponding to Sequence ID 18 was de novo synthesized using gBlocks (Integrated DNA Technologies, Coralville, Iowa) and restriction digested by BglII and

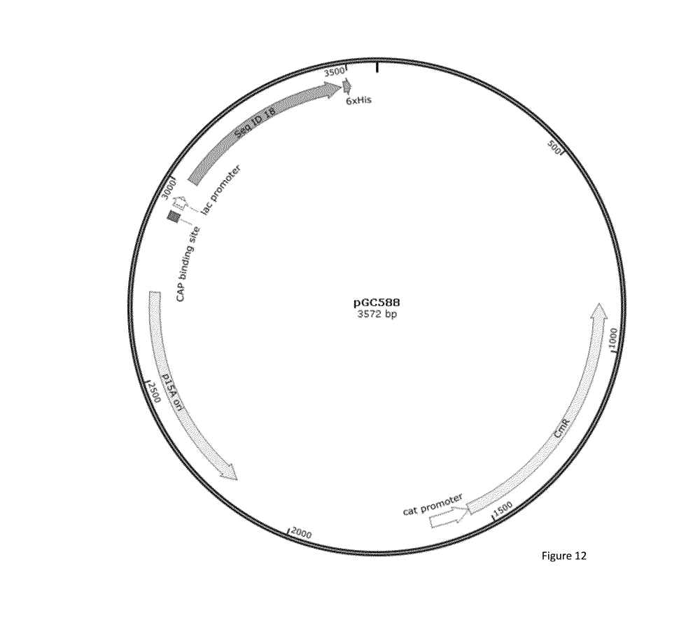

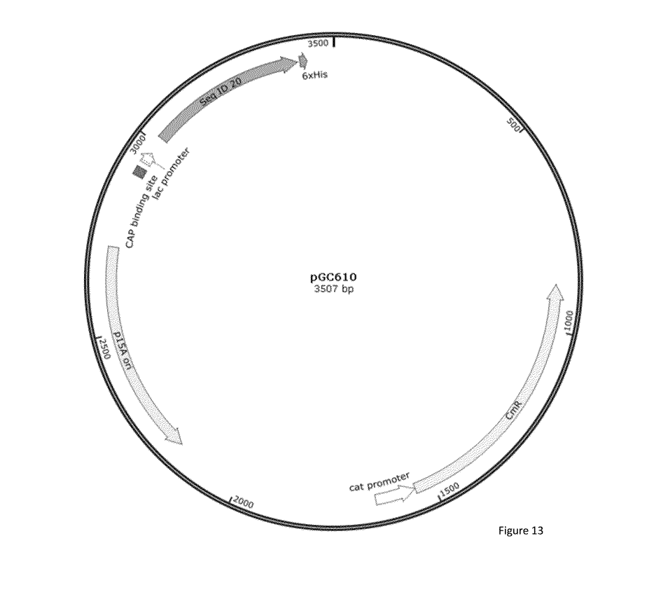

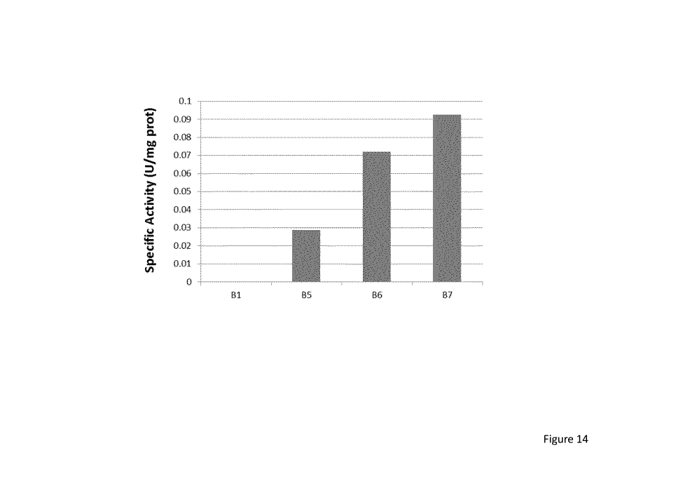

XhoI and ligated into pACYC-based plasmid which was also digested with the same enzymes. The resulting plasmid is designated pGC588 and is shown in FIG. 12. DNA sequence corresponding to Sequence ID 20 was amplified by PCR using the primers with Sequence ID 22 and Sequence ID 23. The amplified DNA was restriction digested using BglII and XhoI and ligated into pACYC-based plasmid which was also digested with the same enzymes. The resulting plasmid is designated pGC610 and is shown in FIG. 13. The plasmids pGC432 and pGC532 (B5), pGC432 and pGC588 (B6) and pGC432 and pGC610 (B7) were transformed into E. coli BW25113 along with the empty plasmid control (B1). The cells harboring these plasmids were grown in medium that contains M9 minimal medium (50%) and LB (50%) and supplemented with glucose. The plasmids were maintained by the addition of 100 mg/L of ampicillin and 50 mg/L of chloramphenicol. Cells from mid-exponential phase were harvested and washed in 0.1 M Tris-HCl buffer (pH 8). The resuspended cells were disrupted by sonication and the cell debris removed by centrifugation. The cell extract was used to assay for methylmalonyl-CoA hydrolase activity. The cell extract was added to DTNB (2.7 mM) in 0.1 mM Tris-HCl buffer and 112.2 μM (S)-methylmalonyl-CoA. The activity of the enzyme was followed by the liberation of CoA at 37° C. for five minutes in 96-well plates. See for example, Andrew Skaff D, Miziorko H M, A visible wavelength spectrophotometric assay suitable for high-throughput screening of 3-hydroxy-3-methylglutaryl-CoA synthase, Anal Biochem. 2010 Jan. 1; 396(1):96-102. The assay control was the reaction mixture without (S)-methylmalonyl-CoA, but with the cell extract. The total protein in the cell extract was measured using Bradford assay. One unit (U) of mmCoA hydrolase activity is defined as the amount of enzyme required to produce 1 μmole of CoA in one minute. FIG. 14 shows the activity of methylmalonyl-CoA hydrolase in three engineered bacteria and not in the parent wild-type.

### Example 4

This example demonstrates the functional expression of methylmalonyl-CoA hydrolases in yeast.

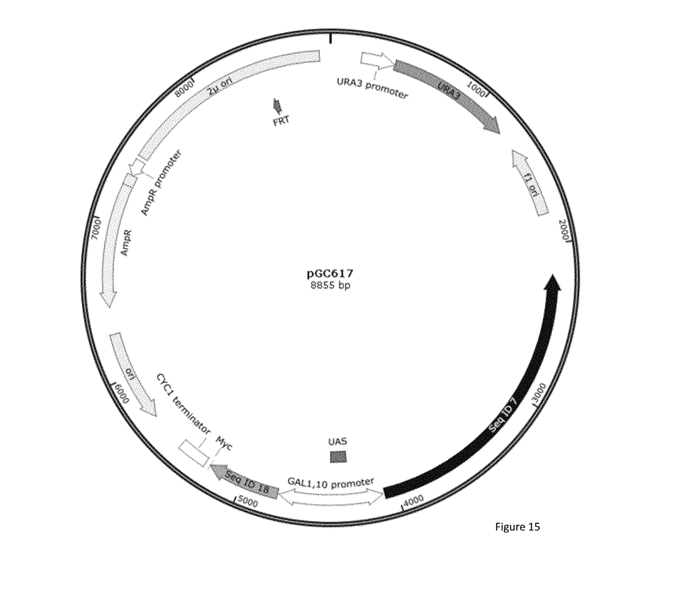

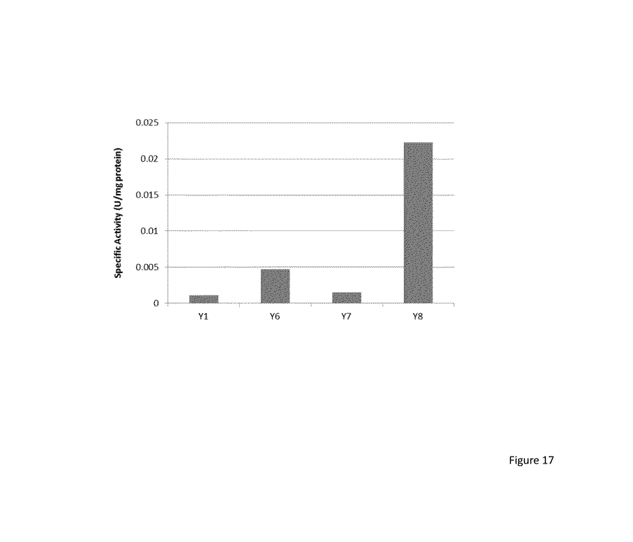

465 bp fragment from pGC588 was liberated by digestion with BamHI and XhoI and was ligated into pGC314 which was also digested with the same enzymes to create pGC617 (FIG. 15). Similarly, 399 by fragment from pGC610 was liberated by digestion with BamHI and XhoI and was ligated into pGC314 which was also digested with the same enzymes to create pGC618 (FIG. 15). The plasmids pGC203 and pGC314 (Y6) pGC203 and pGC617 (Y7) and pGC203 and pGC618 (Y8) were transformed into yeast BY4741 and the resulting transformants were grown in synthetic defined medium lacking uracil and leucine, in the presence of 10 g/L of galactose. The engineered yeast cells were separated from the medium by centrifugation and resuspended in 0.1 M Tris-HCl buffer. The cells were lysed by sonication, debris centrifuged. The methylmalonyl-CoA hydrolase activity was assayed in the cell extracts using a DTNB-based assay that quantifies the liberation of free CoA at 412 nm. One unit (U) of mmCoA hydrolase activity is defined as the amount of enzyme required to produce 1 μmole of CoA in one minute. Methylmalonyl-CoA hydrolase activity could not be detected in the parent wild-type control yeast but was detected in the engineered yeasts with methylmalonyl-CoA hydrolase genes (FIG. 17).

### Example 5

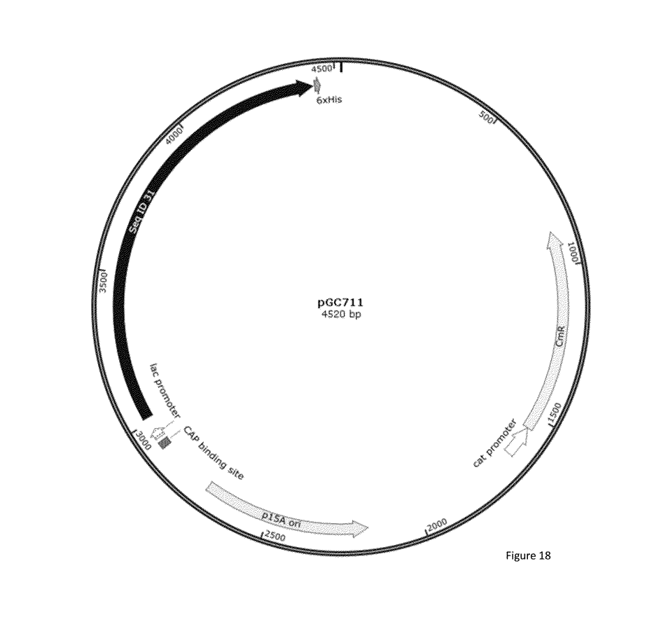

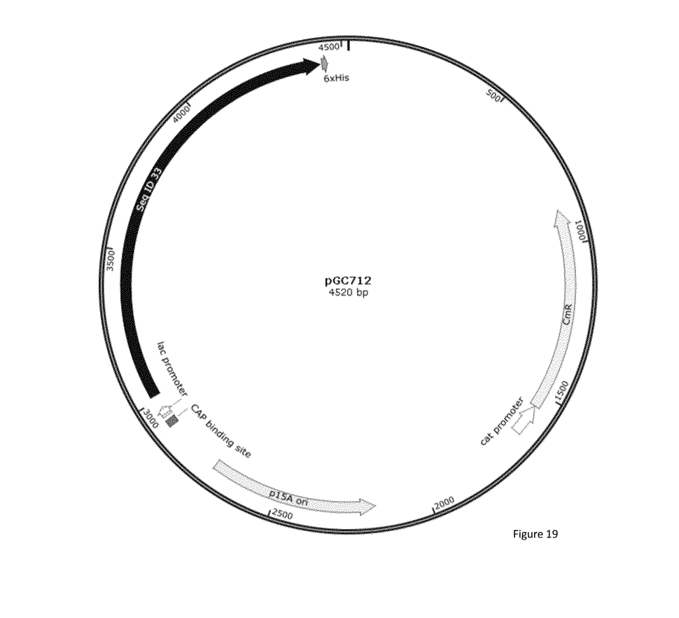

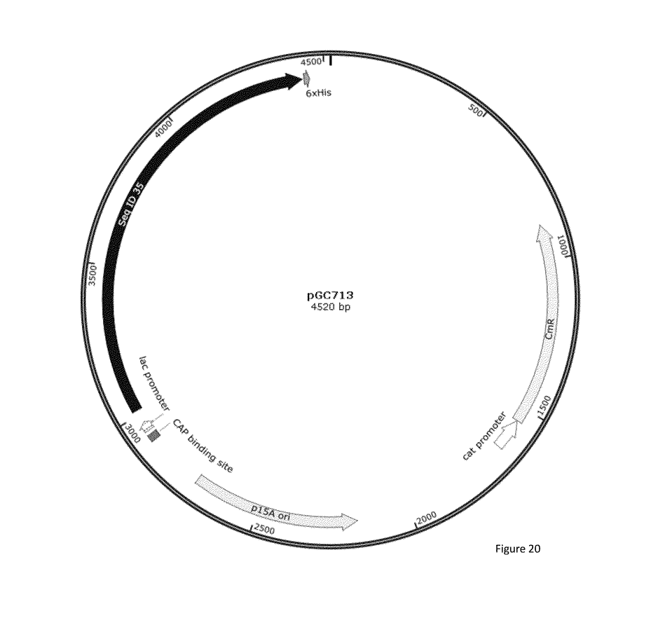

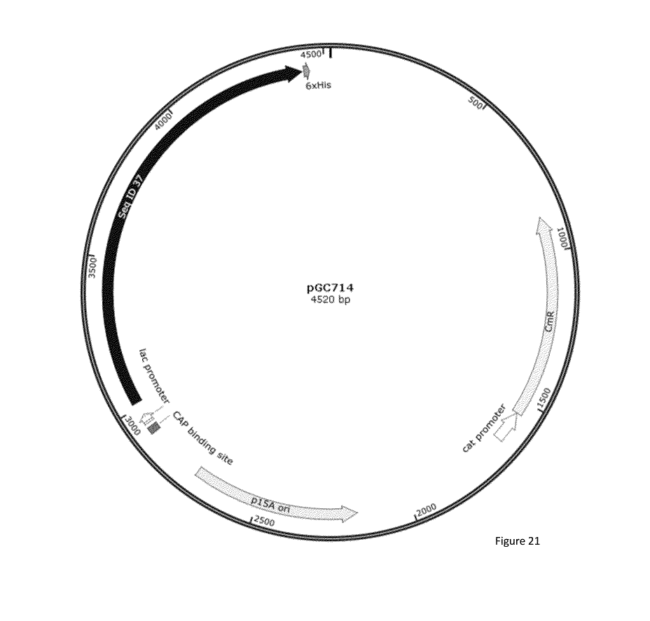

This example demonstrates how the activity of methylmalonyl-CoA hydrolase could be improved by engineering the protein. The sequence corresponding to Sequence ID 10 was able to catalyze the hydrolysis of (S)-methylmalonyl-CoA into methylmalonic acid. In order to improve the activity of the enzyme, critical amino acids were altered using Q5 Site-Directed Mutagenesis kit (New England Biolabs, Ipswich, Mass.). The mutations were introduced by PCR using primers described below and the plasmid pGC532 as a template. Using the primers indicated by Sequence ID 24 and Sequence ID 25, the glutamate 94 of Sequence ID 10 was mutated to serine. The resulting DNA sequence is shown in Sequence ID 31 and the corresponding sequence of the engineered protein is shown in Sequence ID 32. The resulting plasmid is designated pGC711 (FIG. 18). Using primers with sequences corresponding to Sequence ID 26 and Sequence 25, the glutamate 94 of Sequence ID 10 was mutated to valine. The resulting DNA sequence is shown in Sequence ID 33 and the corresponding sequence of the engineered protein is shown in Sequence ID 34. The resulting plasmid is designated pGC712 (FIG. 19). Using primers with sequences corresponding to Sequence ID 27 and Sequence ID 28, the phenylalanine 147 of Sequence ID 10 was mutated to leucine. The resulting DNA sequence is shown in Sequence ID 35 and the corresponding sequence of the engineered protein is shown in Sequence ID 36. The resulting plasmid is designated pGC713 (FIG. 20). Using primers with sequences corresponding to Sequence ID 29 and Sequence ID 30, the Serine 298 of Sequence ID 10 was mutated to alanine. The resulting DNA sequence is shown in Sequence ID 37 and the corresponding sequence of the engineered protein is shown in Sequence ID 38. The resulting plasmid is designated pGC714 (FIG. 21).

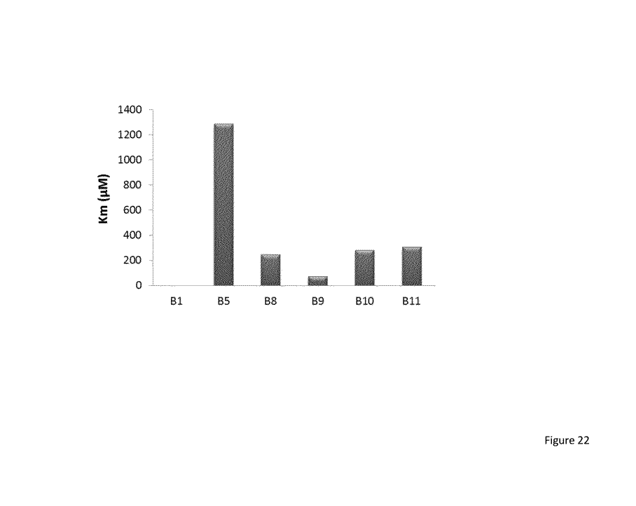

The plasmids pGC711 and pGC432 (B8), pGC712 and pGC432 (B9), pGC713 and pGC432 (B10) and pGC712 and pGC432 (B11) were transformed into BW25113 and these transformants along with B1, empty plasmid control, and B5, harboring the plasmids pGC543 and pGC432 were grown on medium that contains M9 mineral salts (50%) and LB (50%) and ampicillin (100 mg/L) and chloramphenicol (50 mg/L) and glucose as the carbon source. After growth for 8 h, the cells were harvested and resuspended in 0.1 M Tris-HCl (pH 8). Cell extract from these bacterial cells were prepared by sonication and was used to assay for methylmalonyl-CoA hydrolase activity. The assay was performed as described above with 0 μM, 50 μM, 100 μM, 150 μM or 200 μM of (S)-methylmalonyl-CoA in the enzyme mixture. One unit (U) of mmCoA hydrolase activity is defined as the amount of enzyme required to produce 1 μmole of CoA in one minute. The activity as a function of the substrate concentration was plotted as Lineweaver-Burk plot (D L Nelson, M M Cox, Lehninger Principles of Biochemistry WH Freeman Publishing, 2012) to calculate the Michaelis constant (Km). B1 did not have any detectable activity. The value of Km was high for the unengineered methylmalonyl-CoA hydrolase. However, it decreased significantly for the engineered enzymes (see FIG. 22). More specifically as shown in FIG. 22, the Km for B5 was 1289.267 μmole, the Km for B8 was 248.6764 μmole, the Km for B9 was 75.22355 μmole, the Km for B 10 was 284.2105 μmole, and the Km for B11 was 310.5448 μmole.

### Example 6

This example demonstrates the use of engineered enzyme in bacteria for methylmalonic acid production.

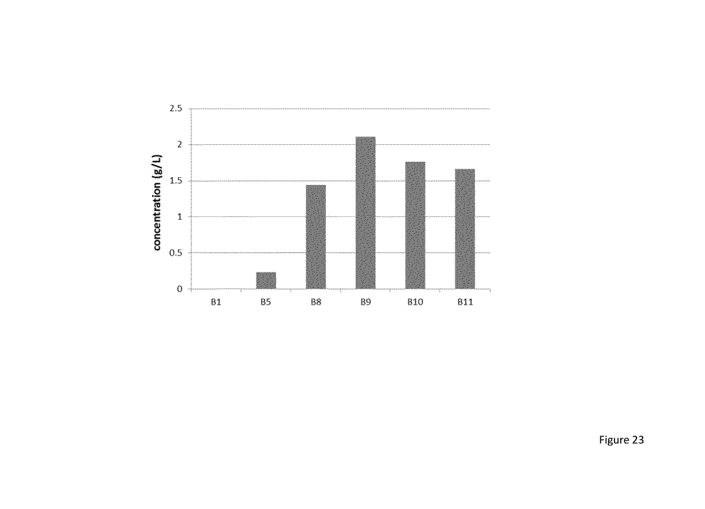

The bacterial cells described in the previous example, B1, B5, B8, B9, B10 and B11 were grown on medium that contains M9 mineral salts (50%) and LB (50%) and ampicillin (100 mg/L) and chloramphenicol (50 mg/L) and glucose as the carbon source. The supernatant was analyzed for methylmalonic acid production. While B1 did not produce any methylmalonic acid, the recombinant bacteria containing the engineered methylmalonyl-CoA hydrolases produced methylmalonic acid. FIG. 23 shows the methylmalonic acid concentration produced by engineered bacteria in the supernatant after 18 h of growth.

### Example 7

This example demonstrates the engineering of yeast cells by the introduction of ATP-generating phosphoenolpyruvate carboxykinase.

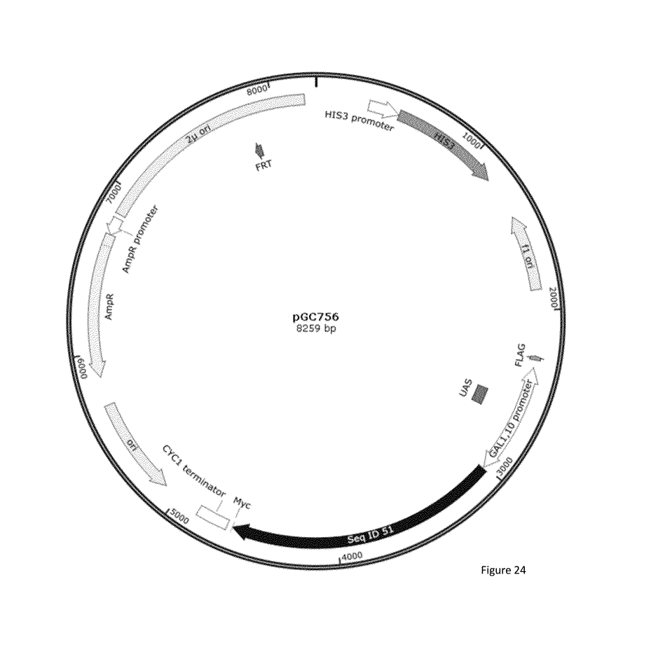

DNA sequence corresponding to SEQ ID 51 is synthesized de novo and is digested with BamHI and XhoI restriction enzymes and is cloned into a yeast/E. coli shuttle vector with ampicillin resistance, histidine marker and Gall promoter for expressing the gene in yeast, which is also digested with the same enzymes. The resulting plasmid is designated pGC756 and is illustrated in FIG. 24. The plasmid is transformed into yeast such as Y6 which is already capable of producing methylmalonic acid. The yeasts are grown in 250 mL shake flasks at 30° C. in 25 mL of synthetic defined lacking uracil, leucine and histidine supplemented with 10 g/L of galactose as carbon source and inducer. Methylmalonic acid is measured in the medium.

### Example 8

This example demonstrates the engineering of yeast cells by the introduction of a NADH transhydrogenase.

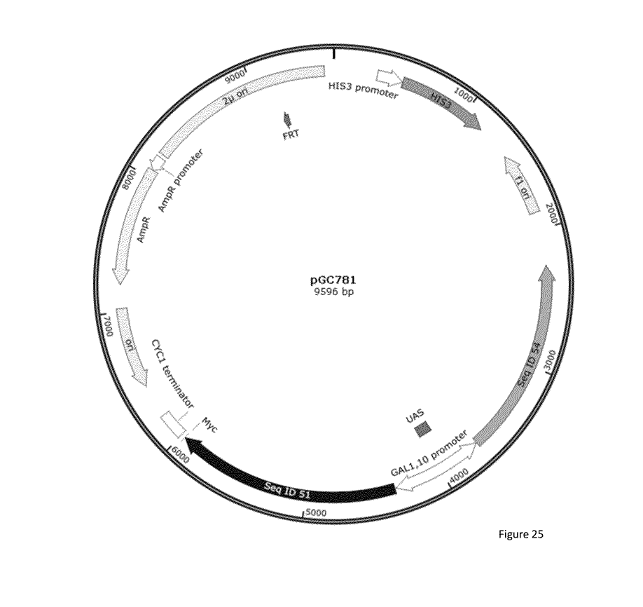

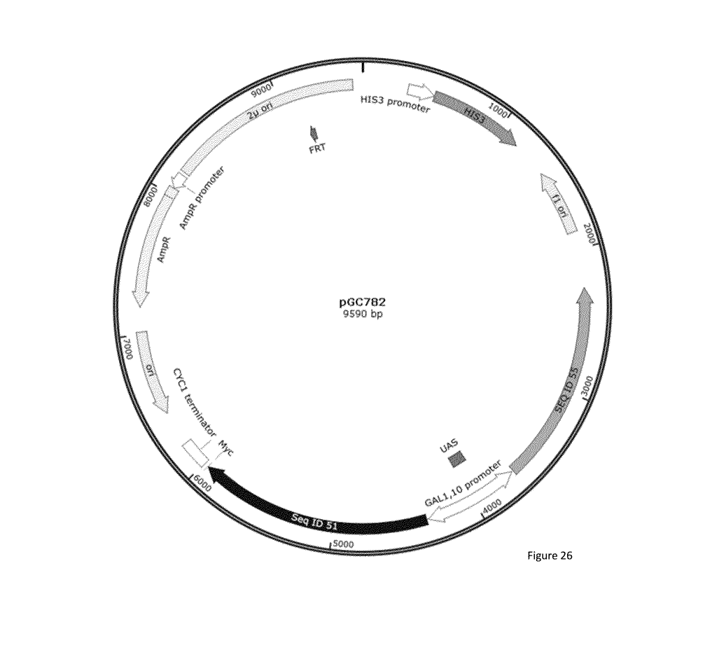

DNA corresponding to SEQ ID 54 and 55 is de novo synthesized with restriction sites for EcoRI and Sad at the 5′ and 3′ ends and is restriction digested with the enzymes. The fragment is cloned into a yeast/E. coli shuttle vector with ampicillin resistance, histidine marker and Gal10 promoter for expressing the gene in yeast which is also digested with the same enzymes. The resulting plasmids are designated pGC781 (FIG. 25) and pGC782 (FIG. 26), respectively. These plasmids are transformed into yeast that already contains a methylmalonic acid pathway. The transformed yeast and those with empty plasmid control are grown in 250 mL shake flasks at 30° C. in 25 mL of synthetic defined lacking uracil, leucine and histidine supplemented with 10 g/L of galactose as carbon source and inducer. Methylmalonic acid is measured in the medium.

### Example 9

The Saccharomyces cerevisiae strain IMX581 (Mans, R., H. M. van Rossum, et al. (2015). CRISPR/Cas9: a molecular Swiss army knife for simultaneous introduction of multiple genetic modifications in Saccharomyces cerevisiae. FEMS Yeast Res 15(2)) has Cas9 nuclease integrated in its chromosome such that it can be used as the host strain for manipulating the genome using CRISPR technology (US20140068797 A1). The guide RNA (gRNA) is expressed from either pMEL or pROS series of plasmids. The genes of the methylmalonic acid pathway are integrated in the chromosome of IMX581 using this technology. The gRNA sequences are designed using Yeastriction online tool (http://yeastriction.tnw.tudelft.nl/#!/). The gRNA sequence is introduced into pMEL plasmid using complementary primers that have 50 bp of homology that are PAGE-purified. The primers are dissolved in distilled water to a final concentration of 100 μM and the primers are mixed in 1:1 molar ratio and the mixture is heated to 95° C. for 5 min and annealed by cooling to room temperature. The primers are mixed with pMEL10 as template and is amplified using Q5 High Fidelity 2X Master Mix (New England BioLabs (Ipswich, Mass.). The PCR product is digested with DpnI for 30 minutes and the PCR product purified on an agarose gel. The protocol for simultaneous integration and deletion is described in Mans et al (supra). Using the protocol, genes that encode for the proteins listed in the table below are integrated into the loci in the S. cerevisiae chromosome. The terminator and promoters that are used to express the genes are also listed in the table. The table also provides metabolic alterations in yeast that are conducive to increased methylmalonic acid production.

TABLE 5
Metabolic engineering of yeast for methylmalonic acid production

| Step | SEQ ID | Target | Promoter | Terminator |
|---|---|---|---|---|
| 1 | 61 | PDC1 | PDC1 | PDC1 |
| 1 | 62 | PDC1 | PDC1 | PDC1 |
| 1 | 63 | PDC1 | PDC1 | PDC1 |
| 2 | 64 | CIT3 | TDH3 | ADH1 |
| 2 | 65 | CIT3 | TDH3 | ADH1 |
| 2 | 66 | CIT3 | TDH3 | ADH1 |
| 3 | 69, 70, 71 | ADH1 | TEF1 | ADH1 |
| | | ADH1 | TEF1 | CYC1 |
| 4 | 72 | GDH1 | PGK1 | CYC1 |
| 11 | 3, 4 | CIT3 | GPD1 | CYC1 |
| | | GAL1 | GPD1 | ADH1 |
| 12 | 10, 21 | GAL10 | PGK1 | CYC1 |
| 15 | 41 | GPD1 | GPD1 | GPD1 |

The engineered yeast hosting the genes of the methylmalonic acid pathway is grown in 250 mL shake flasks at 30° C. in 25 mL of synthetic defined supplemented with 10 g/L of glucose as carbon source. The flasks are shaken at 200 rpm for 24 h. Methylmalonic acid is measured in the supernatant.

A number of embodiments have been described but a person of skill understands that still other embodiments are encompassed by this disclosure. It will be appreciated by those skilled in the art that changes could be made to the embodiments described herein without departing from the broad inventive concepts thereof. It is understood, therefore, that this disclosure and the inventive concepts are not limited to the particular embodiments disclosed, but are intended to cover modifications within the spirit and scope of the inventive concepts including as defined in the appended claims. Accordingly, the foregoing description of various embodiments does not necessarily imply exclusion. For example, “some” embodiments or “other” embodiments may include all or part of “some,” “other,” “further,” and “certain” embodiments within the scope of the invention. Non-exclusive examples of additional embodiments are provided below.

### Additional Embodiments

1. A microorganism engineered to produce or overproduce a target chemical chosen from methylmalonic acid and derivatives thereof.
2. A microorganism according to embodiment 1, wherein the microorganism is chosen from bacteria, yeast and filamentous fungus.
3. A microorganism according to embodiment 1 or 2, wherein the microorganism is also engineered to secrete the target chemical.
4. A microorganism according to embodiment 3, wherein the microorganism is engineered to express or overexpress one or more components of a transporter system capable of secreting the target chemical.
5. A microorganism according to any of embodiments 1-4, wherein the derivatives are chosen from 2-methyl 1,3-propanediol, 3-hydroxy 2-methylpropanoic acid, 2-methyl 1,3-propanediamine, esters of 2-methyl 1,3-propanediol, and esters of methylmalonic acid.
6. A microorganism according to any of embodiments 1-5, wherein the microorganism comprises at least one exogenous nucleic acid sequence encoding at least one polypeptide for converting a first intermediate in a pathway to make the target chemical into a second intermediate or into the target chemical.
7. A microorganism according to embodiment 6, wherein the at least one polypeptide is at least one enzyme capable of facilitating a step in a pathway for producing methylmalonic acid from propanoyl-CoA or a compound from which propanoyl-CoA can be produced.
8. A microorganism according to embodiment 7, wherein the at least one polypeptide comprises an activity chosen from one or more of: threonine dehydratase (EC 4.3.1.19), methionine-γ-lyase (Ec 4.4.1.11), 2-oxobutanoate formate-lyase (EC 2.3.1.-), 2-oxobutanoate synthase (EC 1.2.7.2), branched-chain 2-oxo acid dehydrogenases (EC 1.2.4.4), D-lactate dehydrogenase (EC 1.1.1.28), glyoxylase III (EC 4.2.1.130), glyoxylase I (EC 4.4.1.4), lactate CoA transferase (EC 2.8.3.-), acetyl-CoA synthetase (EC 6.2.1.1), acetaldehyde dehydrogenase (EC 1.2.1.10), lactoyl-Coa dehydratase (EC 4.2.1.54), acryloyl-CoA reductase (EC 1.3.1.95), propanoyl-CoA carboxylase (EC 6.4.1.3), and methylmalonyl-CoA hydrolase (EC 3.1.2.17).
9. A microorganism according to embodiment 6, wherein at least one polypeptide is an enzyme capable of facilitating a step in a pathway for producing methylmalonic acid from succinyl-CoA.
10. A microorganism according to embodiment 9, wherein the at least one polypeptide comprises an activity chosen from one or more of: methylmalonyl-CoA mutase (EC 5.4.99.2), methylmalonyl-CoA epimerase (EC 5.1.99.1), and methylmalonyl-CoA hydrolase (EC 3.1.2.17).
11. A microorganism according to embodiment 6, wherein the at least one polypeptide is at least one enzyme capable of facilitating a step in a pathway for producing methylmalonic acid from L-glutamate.
12. A microorganism according to embodiment 11, wherein the at least one polypeptide comprises an activity chose from one or more of: glutamate mutase (EC 5.4.99.1), 3-methylaspartate transaminase (EC 2.6.1.-), 3-oxo acid decarboxylase (EC 4.1.1.-), methylmalonic semialdehyde dehydrogenase (EC 1.2.1.27), and aldehyde dehydrogenases (EC 1.2.1.-).
13. A microorganism according to any of embodiments 1-3, wherein the microorganism is engineered to produce one or more of: methylmalonic semialdehyde, 3-hydroxy-2-methylpropanoic acid, 2-methylpropane-1,3-diol, and 2-methylpropane-1,3-diamine.
14. A microorganism according to any of embodiments 8, 10, or 12 wherein the at least one polypeptide may also comprise an activity chosen from alcohol dehydrogenase (EC 1.1.1.-), transaminases (EC 2.6.1.-) and alcohol acyl transferases (EC 2.3.1.-).
15. A microorganism having a cytoplasm chosen from yeast and fungi which is further engineered to produce the target chemical in the cytoplasm.
16. A method, comprising: using an engineered microorganism to produce a target chemical chosen from methylmalonic acid and derivates thereof.
17. A method according to embodiment 16, wherein the microorganism is engineered according to any of embodiments 1-15, 33, 36 and 37.
18. A method, comprising: using an engineered microorganism to secrete a target chemical chosen from methylmalonic acid and derivatives thereof.
19. A method according to embodiment 18, wherein the microorganism is engineered to express or overexpress one or more components of a transporter system capable of secreting the target chemical.
20. A method according to embodiment 18, wherein the microorganism is engineered according to any of embodiments 1, 2, 4-15. 36 and 37.
21. A method of producing a target chemical chosen from methylmalonic acid and derivatives thereof, comprising:
   a. contacting a microorganism with a compound chosen from 2-oxobutanoic acid and compounds from which 2-oxobutanoic acid can be produced in one or more steps, wherein the microorganism expresses:
      i. a first polypeptide that facilitates the conversion of 2-oxobutanoate to propanoyl-CoA;
      ii. a second polypeptide that facilitates the conversion of propanoyl-CoA to (S)-methylmalonyl-CoA; and,
      iii. a third polypeptide chosen from:
         1. polypeptides that facilitate the conversion of (S)-methylmalonyl-CoA to methylmalonate; and,
         2. polypeptides that facilitate the conversion of (S)-methylmalonyl to (R)-methylalonyl-CoA.
   b. culturing the microorganism under conditions whereby methylmalonate or methylmalonic acid is produced; and,
   c. harvesting the methylmalonate or methylmalonic acid.
22. A method according to embodiment 21, wherein the compound is methionine, threonine, or a compound from which methionine, threonine, or a combination thereof can be produced in one or more steps, wherein the microorganism further expresses at least one of a fourth polypeptide that facilitates the conversion of methionine to 2-oxobutanoate and a fifth polypeptide that facilitates the conversion of threonine to 2-oxobutanoate.
23. A method according to embodiments 21 or 22, wherein if the third polypeptide is a polypeptide that facilitates the conversion of (S)-methylmalonyl-CoA to (R)-methylmaloyl-CoA, then the microorganism also expresses a sixth polypeptide chosen from: polypeptides that facilitate the transformation of (R)-methylmalonyl-CoA to methylmalonate and polypeptides that facilitate the transformation of (R)-methylmalonyl-CoA to methylmalonic semialdehyde.
24. A method according to embodiment 23, wherein if the sixth polypeptide is a polypeptide that facilitates the transformation of (R)-methylmalonyl-CoA to methylmalonic semialdehyde, then the microorganism also facilitates the transformation of methylmalonic semialdehyde to methylmalonate.
25. A method according to embodiment 21, wherein the compound is pyruvate or a compound from which pyruvate may be produced in one or more steps, wherein the microorganism further expresses:
   a. a fourth polypeptide that facilitates the reduction of pyruvate to D-lactate;
   b. a fifth polypeptide that facilitates the conversion of D-lactate to R-lactoyl-CoA;
   c. a sixth polypeptide that facilitates the dehydration of R-Lactoyl-CoA to acryloyl-CoA; and,
   d. a seventh polypeptide that facilitates the reduction of acryloyl-CoA to propanoyl-CoA.
26. A method according to any of embodiments 21-25, wherein the microorganism is contacted by a carbon source chosen from one or more of: glucose, fructose, sucrose, xylose, arabinose, fatty acids, cellulose, glycerol, glucose oligomers, methane and carbon dioxide.
27. A method of producing methylmalonic acid or derivatives thereof, comprising:
   a. contacting a microorganism with a carbon source chosen from pyruvate and compounds from which pyruvate may be made in one or more steps, wherein the microorganism expresses:
      i. a first polypeptide that facilitates the conversion of succinyl-CoA to R-methylmalonyl-CoA;
      ii. a second polypeptide chosen from polypeptides that facilitate the epimerization of R-methylmalonyl-CoA to S-methylmalonyl-CoA, polypeptides that facilitate the conversion of R-methylmalonyl-CoA to methylmalonic semialdehyde, and combinations thereof; and,
      iii. a third polypeptide chosen from:
         1. polypeptides that facilitate the conversion of S-methylmalonyl-CoA to methylmalonate, if the second polypeptide is or includes a polypeptide that facilitates the epimerization of R-methylmalonyl-CoA to S-methylmalonyl-CoA;
         2. polypeptides that facilitate the conversion of methylmalonic semialdehyde to methylmalonate, polypeptides that facilitate the conversion of methylmalonic semialdehyde to 3-hydroxy-2-methylpropanoic acid, polypeptides that facilitate the conversion of methylmalonic semialdehyde to 2-methylpropane-1,3-diol, polypeptides that facilitate the conversion of methylmalonic semialdehyde to 2-methylpropane-1,3-diamine, if the second polypeptide is or includes a polypeptide that facilitates the conversion of R-methylmalonyl-CoA to S-methylalonic semialdehyde; and,
         3. combinations thereof;
   b. culturing the microorganism under conditions whereby methymalonic acid is produced; and,
   c. harvesting the target chemical.
28. A method according to embodiment 27, wherein the microorganism expresses a polypeptide that facilitates the conversion of methylmalonic semialdehyde to 2-methylpropane-1,3-diol, and further expresses a fourth polypeptide that facilitates the conversion of the 1,3-diol to a corresponding ester.
29. A method according to embodiment 27, wherein the microorganism has mitochondria and a cytosol and the microorganism is engineered to produce the target chemical in the cytosol.
30. A method according to embodiment 29, wherein a signal sequence directing the second polypeptide into the mitochondria is deleted.
31. A method according to embodiment 27, wherein the microorganism expresses a polypeptide that facilitates the conversion of methylmalonic semialdehyde to 3-hydroxy-2-methylpropanoic acid, and further expresses a fourth polypeptide that facilitates dehydration of 3-hydroxy-2-methylpropanoic acid to 2-methylprop-2-enoic acid and a fifth polypeptide corresponding to a transporter for excreting 2-methylprop-2-enoic acid from the microorganism.
32. A method for producing methylmalonic acid or derivatives thereof, comprising:
   a. contacting a microorganism with L-glutamate, herein the microorganism expresses:
      i. a first polypeptide that facilitates the conversion of glutamate to 3-methylaspartate;
      ii. a second polypeptide that facilitates the deamination of 3-methylaspartate to methyloxaloacetate;
      iii. a third polypeptide that facilitates the decarboxylation of methyloxaloacetate to methylmalonic semialdehyde; and,
      iv. a fourth polypeptide that facilitates the conversion of methylmalonic semialdehyde to methylmalonic acid;
   b. culturing the microorganism under conditions whereby methymalonic acid is produced; and,
   c. harvesting the methylmalonic acid.
33. A microorganism according to any of embodiments 1-8, and 11-14 in which aspartate kinase is feedback resistant to threonine.
34. A microorganism according to embodiment 6, wherein the at least one polypeptide is at least one enzyme capable of facilitating a step in a pathway for producing methylmalonic acid from a carbon source or a metabolic intermediate chosen from: pyruvate, methylglyoxal, lactate, threonine, glucose, fructose, sucrose, arabinose, fatty acids, glycerol, valine, leucine, 2-oxobutanoic acid, methane and carbon dioxide.
35. A method according to any of embodiments 16-32 and 37-43, wherein the method further comprises producing the target chemical by the engineered microorganism in a fermenter, and optionally purifying the target chemical.
36. A microorganism according to embodiment 5, wherein the target chemical is 3-hydroxy-2-methylpropanoic acid and the microorganism produces the target chemical by directly converting (R)-methylmalonyl-CoA, (S)-methylmalonyl-CoA, or both to 3-hydroxy-2-methylpropanoic acid by the action of alcohol-forming fatty acyl-CoA reductase (EC 1.2.1.84).
37. A microorganism according to embodiment 36, wherein the microorganism is also engineered to produce a monocarboxylate transporter.
38. A method according to any of embodiments 21, 22 or 25, wherein 3-hydroxy-2-methylpropanoic acid is produced.
39. A method according to embodiment 38, wherein the microorganism expresses at least one of a polypeptide that facilitates the conversion of (R)-methylmalonyl-CoA to 3-hydroxy-2-methylpropanoic acid and polypeptide that facilitates the conversion of (S)-methylmalonyl-CoA to 3-hydroxy-2-methylpropanoic acid.
40. A method according to embodiment 39, wherein the microorganism also produces a monocarboylate transporter.
41. A method of producing 3-hydroxy-2-methylpropanoic acid, comprising:
   a. contacting a microorganism with a carbon source chosen from pyruvate and compounds from which pyruvate may be made in one or more steps, wherein the microorganism expresses:
      i. A first polypeptide that facilitates the conversion of succinyl-CoA to R-methylmalonyl-CoA; optionally,
      ii. A second polypeptide chosen from polypeptides that facilitate the epimerization of R-methylmalonyl-CoA to S-methylmalonyl-CoA; and,
      iii. A third polypeptide chosen from: polypeptides that facilitate the conversion of S-methylmalonyl-CoA to 3-hydroxy-2-methylpropanoic acid and polypeptides that facilitate the conversion of R-methylmalonyl-CoA to 3-hydroxy-2-methylpropanoic acid;
   b. Culturing the microorganism under conditions whereby 3-hydroxy-2-methylpropanoic acid is produced; and,
   c. Harvesting 3-hydroxy-2-methylpropanoic acid.
42. A method according to embodiment 41, wherein the microorganism further expresses a fourth polypeptide corresponding to a transporter for excreting 3-hydroxy-2-methylpropanoic acid from the microorganism.
43. A method according to claim 42 wherein the transporter is monocarboxylate transporter.
44. A non-natural microorganism (bacteria, yeast or fungus) that is capable of producing methylmalonic acid and/or esters thereof
45. A microorganism of embodiment 44, which contains a metabolic pathway that allows it produce more methylmalonic acid.
46. A microorganism of embodiment 44, which is engineered to produce a non-natural methylmalonyl-CoA hydrolase—Step 12 (e.g. Seq ID 10, 32, 34, 36 or Seq ID 19 with at least one mutation at positions I39, M45, V60, K71 and V125), that have higher specificity to methylmalonyl-CoA
47. A microorganism of embodiment 44, which also is engineered to produce enzymes that facilitite:
   a. Step 11 (Seq ID 3 and 4) and Step 6 (e.g. Seq ID 44, 45, 46) and Step 8 (e.g. Seq ID 56, 57), or
   b. Step 11 (e.g. Seq ID 3 and 4) and Step 7 (e.g. Seq ID 58) and Step 8 (e.g. Seq ID 56, 57), or
   c. Step 11 (e.g. Seq ID 3 and 4) and Step 6 (e.g. Seq ID 44, 45, 46) and Step 9 (e.g. Seq ID 47, 48, 49), or
   d. Step 11 (e.g. Seq ID 3 and 4) and Step 7 (e.g. Seq ID 58) and Step 9 (e.g. Seq ID 47, 48, 49), or
   e. Step 11 (e.g. Seq ID 3 and 4) and Step 6 (e.g. Seq ID 44, 45, 46) and Step 10, or
   f. Step 11 (e.g. Seq ID 3 and 4) and Step 7 (e.g. Seq ID 58) and Step 10, or
   g. Step 11 (e.g. Seq ID 3 and 4) and Step 15 (e.g. Seq ID 8, 41, 42)
48. A microorganism according to embodiment 46, which expresses enzymes for Step 6 and Step 7 and also feedback resistant aspartate kinase
49. A microorganism according to embodiments 44-48, in which the microorganism expresses an enzyme that facilitates Step 11 and also expresses enzymes that facilitate Step 1, Step 2, Step 3, Step 4, Step 5 and acetyl-CoA synthase 50. A microorganism according to any of claims 44-49, in which the microorganism is a bacteria and the microorganism expresses the enzyme for Step 12 as well as one or both of an enzyme that facilitates Step 13 (e.g. Seq ID 14) and Step 14 (e.g. Seq ID 39)
51. A bacteria according to any of the above which also is capable of or is engineered to do one or more of:
   a. Down-regulation of lactate dehydrogenase
   b. Down-regulation of pyruvate formate-lyase
   c. Down-regulation of pyruvate oxidase
   d. Down-regulation of PEP:PTS
   e. Down-regulation of methylmalonyl-CoA decarboxylase
   f. Introduction of hexokinase (e.g. Seq ID 59, 60)
   g. Introduction of ATP-generating PEP carboxykinase (e.g. Seq ID 50)
   h. Introduction of a dicarboxylic acid transporter selected from Table 3.
52. A yeast according to any of embodiments 1-49, which is capable of or engineered to do one or more:
   a. Down-regulation of pyruvate transporter
   b. Down-regulate pyruvate decarboxylase
   c. Down-regulate alcohol dehydrogenase
   d. Introduction of ATP-generating PEP carboxykinase (Seq ID 50)
   e. Introduction of dicarboxylic acid transporter selected from Table 4.
53. A microorganism according to any of the above in which the genes are introduced either by plasmid or by integrating in the chromosome.
54. A process for growing an engineered microorganism according to any of the above, comprising growing the microorganism under controlled conditions and supplying it with a carbon source for growth and production of methylmalonic acid or esters, thereof and optionally purifying the target chemical.
55. A process according to embodiment 54, wherein the carbon source is chosen from sugars, propanoate, fatty acids, glycerol, amino acids, keto acids, and Cl substrates.
56. A process according to embodiment 54 or 55, wherein the sugars are chosen from glucose, fructose, sucrose, xylose, arabinose and its polymers, the amino acids are chosen from valine, leucine, and isoleucine, the keto acids are chosen from 2-oxobutanoic acid and pyruvate and the Cl substrates are chosen from methane, carbon monoxide and carbon dioxide.
57. A microorganism according to any of the above embodiments, wherein the yeasts chosen from: Candida, Pichia, Kluyveromyces, Saccharomyces, Debaromyces, Hansenula, Pachysolen and Yarrowia; the bacteria are chosen from: Acetobacterium, Aerobacter, Agrobacterium, Alcaligenes, Azotobacter, Bacillus, Clostridium, Corynebacterium, Escherichia, Flavobacterium, Lactobacillus, Micromonospora, Mycobacterium, Nocardia, Propionibacterium, Protaminobacter, Proteus, Pseudomonas, Rhizobium, Salmonella, Serratia, Streptomyces, Streptococcus and Xanthomonas; and, the Fungi are chosen from: Aspergillus, Penicillium, Acremonium, Fusarium, Neospora and Mucor.

What is claimed is:

1. A non-natural microorganism chosen from archaea, bacteria, yeast or fungus which is engineered to produce or overproduce methylmalonic acid.

2. A non-natural microorganism according to claim 1, wherein the microorganism is engineered to overproduce methylmalonic acid.

3. A non-natural microorganism according to claim 1, comprising at least one exogenous gene encoding for a methylmalonyl-CoA hydrolase, wherein the hydrolase is engineered and the engineered hydrolase has a Km for methylmalonyl CoA that is less than the Km for the corresponding wild-type hydrolase.

4. A non-natural microorganism according to claim 3, wherein the Km is less than at least about half the Km of the corresponding wild-type hydrolase.

5. A non-natural microorganism according to claim 1, comprising at least one exogenous gene encoding for a methylmalonyl-CoA hydrolase, wherein the at least one exogenous gene is chosen from a gene having from about 95% to 100% sequence identity to an amino acid sequence chosen from
a. Seq. ID. 10, wherein at least one or more amino acids corresponding to the positions 94, 147 and 298 of Seq ID 10 are mutated such that the amino acid corresponding to position 94 is chosen from valine, serine, alanine, threonine, serine, leucine and isoleucine, the amino acid corresponding to position 147 is chosen from valine, alanine, leucine, glycine amd isoleucine and the amino acid corresponding to position 298 is chosen from alanine and glycine;
b. Seq. ID No. 74, wherein at least one or more amino acids corresponding to the positions 94, 147 and 298 of Seq ID 10 are mutated such that the amino acid corresponding to position 94 is chosen from valine, serine, alanine, threonine, leucine and isoleucine, the amino acid corresponding to position 147 is chosen from valine, alanine, leucine, glycine amd isoleucine and the amino acid corresponding to position 298 is chosen from alanine and glycine;
c. Seq. ID No. 19, wherein at least one or more amino acids corresponding to the positions 39, 45, 60, 71 and 125 of Seq ID 19 are mutated such that the amino acid corresponding to position 39 is chosen from leucine, valine and phenylalanine, the amino acid corresponding to the position 45 is chosen from serine, threonine, tyrosine, lysine and arginine, the amino acid corresponding to the position 60 is chosen from alanine, isoleucine, leucine and phenylalanine, the amino acid corresponding to position 71 is chosen from valine, arginine, glutamine or asparagine, and the amino acid corresponding to position 125 is chosen from glutamate, leucine, isoleucine and aspartate; and,
d. Seq. ID No. 43, wherein at least one or more amino acids corresponding to the positions 34, 40, 55, 66, and 117 of Seq. ID 43 are mutated such that the amino acid corresponding to the position 34 is chosen from leucine, valine and phenylalanine, the amino acid corresponding to the position 40 is chosen from serine, threonine, tyrosine, lysine, methionine and arginine, the amino acid corresponding to position 55 is chosen from valine, isoleucine, leucine and phenylalanine, the amino acid corresponding to position 66 is chosen from lysine, arginine, glutamine and asparagine, the amino acid corresponding to position 117 is chosen from glutamate, leucine, isoleucine and aspartate.

6. A non-natural microorganism according to claim 5, further comprising:
a. a gene encoding an enzyme having from about 95% to 100% sequence identity to an amino acid sequence set forth in SEQ. ID 3 or SEQ. ID 4; and
(i) a gene encoding an enzyme having from about 95% to 100% sequence identity to an amino acid sequence set forth in SEQ ID 8, 41 or 42; and a gene encoding an enzyme that can catalyze at least one of Step 6 or Step 7, and at least one of Step 8, 9 or 10; or
(ii) a gene encoding an enzyme that can catalyze Step 1 Step 2, Step 3, Step 4
b. a gene encoding an enzyme having from about 95% to 100% sequence identity to an amino acid sequence set forth in SEQ. ID 14 or SEQ. ID 39,
wherein at least one of the genes is an exogenous gene.

7. A non-natural microorganism according to claim 1, wherein if the microorganism is a an archaea or a bacteria, the microorganism is engineered to have one or more activities chosen from: down-regulation of lactate dehydrogenase, down-regulation of pyruvate formate-lyase, down-regulation of pyruvate oxidase, down-regulation of PEP:PTS, down-regulation of methylmalonyl-CoA decarboxylase, express or overexpress hexokinase, express or overexpress ATP-generating PEP carboxykinase, and express or overexpress a dicarboxylic acid transporter;

8. A non-natural microorganism according to claim 1, wherein if the microorganism is a yeast, the yeast is engineered to have one or more activities chosen from: down-regulation of pyruvate mitochondrial transporter, down-regulation of pyruvate decarboxylase, down-regulation of alcohol dehydrogenase, express or overexpress formate dehydrogenase, ATP-generating PEP carboxykinase, pyridine transhydrogenase and express or overexpress a dicarboxylic acid transporter.

9. A process for producing methylmalonic acid, comprising growing a microorganism according to claim 1 under controlled conditions; supplying the microorganism with a carbon source for growth and production of methylmalonic acid; and, optionally purifying the methylmalonic acid.

10. A process according to claim 9, wherein the carbon source is chosen from sugars, propanoate, fatty acids, glycerol, amino acids, keto acids, and Cl substrates.

11. A process according to claim 10, wherein the sugars are chosen from glucose, fructose, sucrose, xylose, arabinose and its polymers, the amino acids are chosen from valine, leucine, and isoleucine, the keto acids are chosen from 2-oxobutanoic acid and pyruvate and the C1 substrates are chosen from methane, carbon monoxide and carbon dioxide.

12. A non-natural microorganism according to claim 1, wherein the yeasts are chosen from: Candida, Pichia, Kluyveromyces, Saccharomyces, Debaromyces, Hansenula, Pachysolen and Yarrowia; the bacteria are chosen from: Acetobacterium, Aerobacter, Agrobacterium, Alcaligenes, Azotobacter, Bacillus, Clostridium, Corynebacterium, Escherichia, Flavobacterium, Lactobacillus, Micromonospora, Mycobacterium, Nocardia, Propionibacterium, Protaminobacter, Proteus, Pseudomonas, Rhizobium, Salmonella, Serratia, Streptomyces, Streptococcus and Xanthomonas; the Fungi are chosen from: Aspergillus, Penicillium, Acremonium, Fusarium, Neospora and Mucor; and, the archaea are hydrogenotrophic methanogens.

13. A non-natural microorganism according to claim 1, wherein the microorganism is also engineered to secrete the target chemical by expressing or overexpressing one or more components of a transporter system capable of secreting the target chemical.

14. A non-natural microorganism according to claim 1, wherein the microorganism comprises at least one exogenous nucleic acid sequence encoding at least one polypeptide for converting a first intermediate in a pathway to make the methylmalonic acid into a second intermediate or into the methylmalonic acid, and further wherein the at least one polypeptide is one or more of:
at least one enzyme capable of facilitating a step in a pathway for producing the methylmalonic acid from propanoyl-CoA or a compound from which propanoyl-CoA can be produced;
at least one polypeptide is an enzyme capable of facilitating a step in a pathway for producing the methylmalonic acid from succinyl-CoA; and,
at least one polypeptide is at least one enzyme capable of facilitating a step in a pathway for producing the methylmalonic acid from L-glutamate.

15. A non-natural microorganism according to claim 1, wherein the microorganism comprises at least one exogenous nucleic acid sequence encoding at least one polypeptide for converting a first intermediate in a pathway to make methylmalonic acid into a second intermediate or into the methylmalonic acid, and further wherein the at least one polypeptide comprises an activity chosen from one or more of: threonine dehydratase (EC 4.3.1.19), methionine-γ-lyase (Ec 4.4.1.11), 2-oxobutanoate formate-lyase (EC 2.3.1.-), 2-oxobutanoate synthase (EC 1.2.7.2), branched-chain 2-oxo acid dehydrogenases (EC 1.2.4.4), D-lactate dehydrogenase (EC 1.1.1.28), L-lactate dehydrogenase (EC 1.1.1.27), glyoxylase III (EC 4.2.1.130), glyoxylase I (EC 4.4.1.4), lactate CoA transferase (EC 2.8.3.-), acetyl-CoA synthetase (EC 6.2.1.1), propionyl-CoA synthase (EC 6.2.1.17), acetaldehyde dehydrogenase (EC 1.2.1.10), lactoyl-CoA dehydratase (EC 4.2.1.54), acryloyl-CoA reductase (EC 1.3.1.95), propanoyl-CoA carboxylase (EC 6.4.1.3), and methylmalonyl-CoA hydrolase (EC 3.1.2.17).

16. A non-natural microorganism according to claim 1, wherein the microorganism comprises at least one exogenous nucleic acid sequence encoding at least one polypeptide for converting a first intermediate in a pathway to make the methylmalonic acid into a second intermediate or into the methylmalonic acid, and further wherein the at least one polypeptide comprises an activity chosen from one or more of: methylmalonyl-CoA mutase (EC 5.4.99.2), methylmalonyl-CoA epimerase (EC 5.1.99.1), and methylmalonyl-CoA hydrolase (EC 3.1.2.17).

17. A non-natural microorganism according to claim 1, wherein the at least one polypeptide comprises an activity chose from one or more of: glutamate mutase (EC 5.4.99.1), 3-methylaspartate transaminase (EC 2.6.1.-), 3-oxo acid decarboxylase (EC 4.1.1.-), methylmalonic semialdehyde dehydrogenase (EC 1.2.1.27), and aldehyde dehydrogenases (EC 1.2.1.-).

18. A non-natural microorganism according to claim 1, wherein the microorganism is a yeast or a fungi, and further wherein the microorganism is engineered to produce the methylmalonic acid in the cytoplasm.

19. A method comprising: producing the methylmalonic acid in a fermenter by a microorganism according to claim 1; and, optionally purifying the methylmalonic acid.

20. A methylmalonic acid composition produced by the non-natural microorganism according to claim 1.
